(19) United States
(12) Patent Application Publication
(10) Pub. No.: US 2022/0289685 A1
(43) Pub. Date: Sep. 15, 2022

(54) ANTIMALARIAL HEXAHYDROPYRIMIDINE ANALOGUES

(71) Applicant: UCB BIOPHARMA SRL, Brussels (BE)

(72) Inventors: Teresa De Haro Garcia, Slough, Berkshire (GB); Martin Alexander Lowe, Slough, Berkshire (GB); Malcolm MacCoss, Seabrook Island, SC (US); Richard David Taylor, Slough, Berkshire (GB); Zhaoning Zhu, Slough, Berkshire (GB); Rose Elizabeth Chappell, Abingdon (GB)

(21) Appl. No.: 17/632,336

(22) PCT Filed: Aug. 17, 2020
(86) PCT No.: PCT/EP2020/073011

§ 371 Date: Feb. 2, 2022

(30) Foreign Application Priority Data
Aug. 19, 2019 (GB) 1911865.2
Sep. 24, 2019 (GB) 1913759.5

(51) Int. Cl. C07D 239/22; C07D 407/04; C07D 471/04; C07D 407/14; A61P 33/06; A61K 47/06

(52) U.S. Cl. CPC C07D 239/22; C07D 407/04; C07D 471/04; C07D 407/14; A61P 33/06; A61K 47/06

(57) ABSTRACT

A series of 2-imino-6-methylhexahydropyrimidin-4-one derivatives, and analogues thereof, substituted in the 6-position by an arylcarbonylaminophenyl or heteroarylcarbonylaminophenyl moiety, being potent inhibitors of the growth and propagation of the Plasmodium falciparum parasite in human blood, are beneficial as pharmaceutical agents, especially in the treatment of malaria.

The present invention relates to a class of heterocyclic compounds, and to their use in therapy. More particularly, this invention is concerned with pharmacologically active substituted hexahydropyrimidine derivatives, and analogues thereof. These compounds are potent inhibitors of the growth and propagation of the Plasmodium falciparum parasite in human blood, and are accordingly of benefit as pharmaceutical agents, especially in the treatment of malaria.

Malaria is a mosquito-borne infectious disease, caused by a parasite of the genus Plasmodium, which has devastating consequences. In 2010, an estimated 225 million cases were reported, with 610,000 to 971,000 deaths, approximately 80% of which occurred in sub-Saharan Africa, mostly in young children (aged 5 years or less).

The compounds in accordance with the present invention, being potent inhibitors of the growth and propagation of the P. falciparum parasite in human blood, are therefore beneficial in the treatment of malaria.

In addition, the compounds in accordance with the present invention may be beneficial as pharmacological standards for use in the development of new biological tests and in the search for new pharmacological agents. Thus, the compounds of this invention may be useful as radioligands in assays for detecting pharmacologically active compounds.

Co-pending international patent application no. PCT/EP2019/058249 (published on 18 Oct. 2019 as WO 2019/192992), and co-pending international patent application no. PCT/EP2020/063083 (claiming priority from United Kingdom patent application no. 1906804.8), describe certain classes of heterocyclic compounds which are stated to be potent inhibitors of the growth and propagation of the P. falciparum parasite in human blood, and therefore to be beneficial in the treatment of malaria.

CN-109180670-A discloses iminothiadiazine dioxide derivatives that are stated to be BACE-1 inhibitors useful for treating diseases related to beta-amyloid protein, and especially Alzheimer disease.

WO 2017/142825 describes a family of heterocyclic compounds which are stated to be potent inhibitors of P. falciparum growth in vitro that may be useful for the treatment of malaria.

WO 2017/089453 and WO 2017/144517 describe heterocyclic compounds which are stated to be potent and selective inhibitors of plasmepsin V activity that are beneficial in the treatment of malaria.

WO 2016/172255, WO 2016/118404 and WO 2011/044181 describe certain classes of heterocyclic compounds which are stated to be BACE inhibitors that may be useful for treating Aβ-related pathologies including Alzheimer's disease.

WO 2012/019966 describes 1,4,5,6-tetrahydropyrimidin-2-ylamine derivatives which are stated to have BACE2 inhibitory properties that may be useful in the treatment of metabolic disorders (including type 2 diabetes), and cardiovascular disorders.

WO 2008/103351, WO 2006/065277 and WO 2005/058311 describe a family of heterocyclic compounds that are stated to be aspartyl protease inhibitors. The compounds described in those publications are also stated to be effective in a method of inhibiting inter alia plasmepsins (specifically plasmepsins I and II) for treatment of malaria.

WO 2006/041404 describes a family of heterocyclic compounds that are stated to be inhibitors of Beta site APP (amyloid precursor protein) Cleaving Enzyme (BACE). The compounds described in that publication are also stated to be effective in a method of modulating BACE activity; and in methods of treating or preventing an amyloid-β-protein-related (Aβ-related) pathology, including Downs syndrome and Alzheimer disease.

The present invention provides a compound of formula (I) or an N-oxide thereof, or a pharmaceutically acceptable salt thereof:

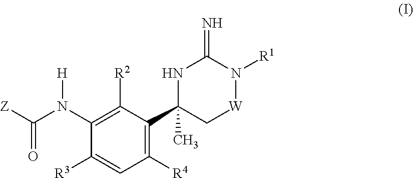

wherein

W represents C(O) or S(O)₂;

Z represents aryl or heteroaryl, either of which groups may be optionally substituted by one or more substituents;

R¹ represents C₃₋₇ cycloalkyl, aryl(C₁₋₆)alkyl, C₃₋₇ heterocycloalkyl, C₃₋₇ heterocycloalkyl(C₁₋₆)alkyl, C₄₋₉ heterobicycloalkyl, C₄₋₉ spiroheterocycloalkyl or heteroaryl(C₁₋₆)alkyl, any of which groups may be optionally substituted by one or more substituents; and

R², R³ and R⁴ independently represent hydrogen, halogen or trifluoromethyl.

The compounds in accordance with the present invention are encompassed within the broadest generic scope of WO 2016/172255, WO 2011/044181, WO 2008/103351, WO 2006/065277, WO 2005/058311 and WO 2006/041404. There is, however, no specific disclosure in any of those publications of a compound of formula (I) as defined above, or a pharmaceutically acceptable salt thereof.

The present invention also provides a compound of formula (I) as defined above, or a pharmaceutically acceptable salt thereof.

The present invention also provides a compound of formula (I) as defined above or an N-oxide thereof, or a pharmaceutically acceptable salt thereof, for use in therapy.

The present invention also provides a compound of formula (I) as defined above or an N-oxide thereof, or a pharmaceutically acceptable salt thereof, for use in the treatment and/or prevention of malaria.

The present invention also provides a method for the treatment and/or prevention of malaria which comprises administering to a patient in need of such treatment an effective amount of a compound of formula (I) as defined above or an N-oxide thereof, or a pharmaceutically acceptable salt thereof.

The present invention also provides the use of a compound of formula (I) as defined above or an N-oxide thereof, or a pharmaceutically acceptable salt thereof, for the manufacture of a medicament for the treatment and/or prevention of malaria.

Where any of the groups in the compounds of formula (I) above is stated to be optionally substituted, this group may be unsubstituted, or substituted by one or more substituents. Typically, such groups will be unsubstituted, or substituted by one, two or three substituents, generally by one or two substituents.

For use in medicine, the salts of the compounds of formula (I) will be pharmaceutically acceptable salts. Other salts may, however, be useful in the preparation of the compounds of use in the invention or of their pharmaceutically acceptable salts. Standard principles underlying the selection and preparation of pharmaceutically acceptable salts are described, for example, in Handbook of Pharmaceutical Salts: Properties, Selection and Use, ed. P. H. Stahl & C. G. Wermuth, Wiley-VCH, 2002.

Suitable alkyl groups which may be present on the compounds of use in the invention include straight-chained and branched C₁₋₆ alkyl groups, for example C₁₋₄ alkyl groups. Typical examples include methyl and ethyl groups, and straight-chained or branched propyl, butyl and pentyl groups. Particular alkyl groups include methyl, ethyl, n-propyl, isopropyl, n-butyl, sec-butyl, isobutyl, tert-butyl, 2,2-dimethylpropyl and 3-methylbutyl. Derived expressions such as “C₁₋₆ alkoxy”, “C₁₋₆ alkylthio”, “C₁₋₆ alkylsulfonyl” and “C₁₋₆ alkylamino” are to be construed accordingly.

The term “C₃₋₇ cycloalkyl” as used herein refers to monovalent groups of 3 to 7 carbon atoms derived from a saturated monocyclic hydrocarbon, and may comprise benzo-fused analogues thereof. Suitable C₃₋₇ cycloalkyl groups include cyclopropyl, cyclobutyl, benzocyclobutenyl, cyclopentyl, indanyl, cyclohexyl and cycloheptyl.

The term “aryl” as used herein refers to monovalent carbocyclic aromatic groups derived from a single aromatic ring or multiple condensed aromatic rings. Suitable aryl groups include phenyl and naphthyl, preferably phenyl.

Suitable aryl(C₁₋₆)alkyl groups include benzyl, phenylethyl, phenylpropyl and naphthylmethyl.

The term “C₃₋₇ heterocycloalkyl” as used herein refers to saturated monocyclic rings containing 3 to 7 carbon atoms and at least one heteroatom selected from oxygen, sulphur and nitrogen, and may comprise benzo-fused analogues thereof. Suitable heterocycloalkyl groups include oxetanyl, azetidinyl, tetrahydrofuranyl, dihydrobenzo-furanyl, dihydrobenzothienyl, pyrrolidinyl, indolinyl, isoindolinyl, oxazolidinyl, thiazolidinyl, isothiazolidinyl, imidazolidinyl, tetrahydropyranyl, chromanyl, dioxanyl, tetrahydrothiopyranyl, piperidinyl, 1,2,3,4-tetrahydroquinolinyl, 1,2,3,4-tetrahydro-isoquinolinyl, piperazinyl, 1,2,3,4-tetrahydroquinoxalinyl, hexahydro-[1,2,5]thiadiazolo-[2,3-a]pyrazinyl, homopiperazinyl, morpholinyl, benzoxazinyl, thiomorpholinyl, azepanyl, oxazepanyl, diazepanyl, thiadiazepanyl and azocanyl.

The term “C₄₋₉ heterobicycloalkyl” as used herein refers to monovalent groups of 4 to 9 carbon atoms derived from a saturated bicyclic hydrocarbon, comprising one or more heteroatoms selected from oxygen, sulphur and nitrogen. Typical heterobicyclo-alkyl groups include 3-azabicyclo[3.1.0]hexanyl, 2-oxa-5-azabicyclo[2.2.1]heptanyl, 7-oxabicyclo[2.2.1]hexanyl, 6-azabicyclo[3.2.0]heptanyl, 3-azabicyclo[3.1.1]heptanyl, 6-oxa-3-azabicyclo[3.1.1]heptanyl, 3-azabicyclo[4.1.0]heptanyl, 2-oxabicyclo[2.2.2]-octanyl, quinuclidinyl, 2-oxa-5-azabicyclo[2.2.2]octanyl, 3-azabicyclo[3.2.1]octanyl, 8-oxabicyclo[3.2.1]octanyl, 8-azabicyclo[3.2.1]octanyl, 3-oxa-8-azabicyclo[3.2.1]octanyl, 3,8-diazabicyclo[3.2.1]octanyl, 3,6-diazabicyclo[3.2.2]nonanyl, 3-oxa-7-azabicyclo-[3.3.1]nonanyl, 3,7-dioxa-9-azabicyclo[3.3.1]nonanyl and 3,9-diazabicyclo[4.2.1]-nonanyl.

The term “C₄₋₉ spiroheterocycloalkyl” as used herein refers to saturated bicyclic ring systems containing 4 to 9 carbon atoms and at least one heteroatom selected from oxygen, sulphur and nitrogen, in which the two rings are linked by a common atom. Suitable spiroheterocycloalkyl groups include 5-azaspiro[2.3]hexanyl, 5-azaspiro[2.4]-heptanyl, 2-oxaspiro[3.3]heptanyl, 2-azaspiro[3.3]heptanyl, 2-oxa-6-azaspiro[3.3]-heptanyl, 3-oxa-6-azaspiro[3.3]heptanyl, 6-thia-2-azaspiro[3.3]heptanyl, 2-oxa-6-azaspiro[3.4]octanyl, 2-oxa-6-azaspiro[3.5]nonanyl, 7-oxa-2-azaspiro[3.5]nonanyl, 2-oxa-7-azaspiro[3.5]nonanyl and 2,4,8-triazaspiro[4.5]decanyl.

The term “heteroaryl” as used herein refers to monovalent aromatic groups containing at least five atoms derived from a single ring or multiple condensed rings, wherein one or more carbon atoms have been replaced by one or more heteroatoms selected from oxygen, sulfur and nitrogen. Suitable heteroaryl groups include furyl, benzofuryl, dibenzofuryl, thienyl, benzothienyl, thieno[2,3-c]pyrazolyl, thieno[3,2-c]-pyridinyl, dibenzothienyl, pyrrolyl, indolyl, pyrrolo[2,3-b]pyridinyl, pyrrolo[3,2-c]-pyridinyl, pyrrolo[3,4-b]pyridinyl, pyrazolyl, pyrazolo[1,5-a]pyridinyl, pyrazolo[3,4-b]-pyridinyl, pyrazolo[3,4-d]pyrimidinyl, indazolyl, 4,5,6,7-tetrahydroindazolyl, oxazolyl, benzoxazolyl, isoxazolyl, thiazolyl, benzothiazolyl, isothiazolyl, imidazolyl, benzimidazolyl, imidazo[2,1-b]thiazolyl, imidazo[1,2-a]pyridinyl, imidazo[1,5-a]-pyridinyl, imidazo[4,5-b]pyridinyl, purinyl, imidazo[1,2-a]pyrimidinyl, imidazo[1,2-a]-pyrazinyl, oxadiazolyl, thiadiazolyl, triazolyl, [1,2,4]triazolo[1,5-a]pyridinyl, [1,2,4]triazolo[1,5-a]pyrimidinyl, benzotriazolyl, tetrazolyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl and chromenyl.

The term “halogen” as used herein is intended to include fluorine, chlorine, bromine and iodine atoms, typically fluorine, chlorine or bromine.

The absolute stereochemical configuration of the chiral carbon atom in the W-containing six-membered ring of the compounds according to the invention is as depicted in formula (I) above. Generally, the compounds in accordance with the invention are at least 51% enantiomerically pure (by which it is meant that a sample thereof comprises a mixture of enantiomers containing 51% or more of the enantiomer depicted in formula (I) and 49% or less of the opposite antipode). Typically, the compounds in accordance with the invention are at least 60% enantiomerically pure. Appositely, the compounds in accordance with the invention are at least 75% enantiomerically pure. Suitably, the compounds in accordance with the invention are at least 80% enantiomerically pure. More suitably, the compounds in accordance with the invention are at least 85% enantiomerically pure. Still more suitably, the compounds in accordance with the invention are at least 90% enantiomerically pure. Even more suitably, the compounds in accordance with the invention are at least 95% enantiomerically pure. Preferably, the compounds in accordance with the invention are at least 99% enantiomerically pure. Ideally, the compounds in accordance with the invention are at least 99.9% enantiomerically pure.

Where the compounds of formula (I) have one or more additional asymmetric centres, they may accordingly exist as enantiomers. Where the compounds in accordance with the invention possess one or more additional asymmetric centres, they may also exist as diastereomers. The invention is to be understood to extend to the use of all such enantiomers and diastereomers, and to mixtures thereof in any proportion, including racemates. Formula (I) and the formulae depicted hereinafter are intended to represent all individual stereoisomers and all possible mixtures thereof, unless stated or shown otherwise. In addition, compounds of formula (I) may exist as tautomers, for example keto (CH₂C═O)↔enol (CH═CHOH) tautomers or amide (NHC═O)↔hydroxyimine (N═COH) tautomers or imide (NHC═NH)↔aminoimine (N═CNH₂) tautomers. Formula (I) and the formulae depicted hereinafter are intended to represent all individual tautomers and all possible mixtures thereof, unless stated or shown otherwise. In addition, under certain circumstances, e.g. where R² represents halogen, compounds of formula (I) may exist as atropisomers. Formula (I) and the formulae depicted hereinafter are intended to represent all individual atropisomers and all possible mixtures thereof, unless stated or shown otherwise.

It is to be understood that each individual atom present in formula (I), or in the formulae depicted hereinafter, may in fact be present in the form of any of its naturally occurring isotopes, with the most abundant isotope(s) being preferred. Thus, by way of example, each individual hydrogen atom present in formula (I), or in the formulae depicted hereinafter, may be present as a ¹H, ²H (deuterium; D) or ³H (tritium; T) atom, preferably ¹H. Similarly, by way of example, each individual carbon atom present in formula (I), or in the formulae depicted hereinafter, may be present as a ¹²C, ¹³C or ¹⁴C atom, preferably ¹²C.

In a first embodiment, W represents C(O). In a second embodiment, W represents S(O)₂.

In a first embodiment, the present invention provides a compound of formula (IA) or an N-oxide thereof, or a pharmaceutically acceptable salt thereof.

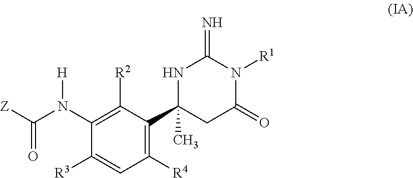

wherein

Z, R¹, R², R³ and R⁴ are as defined above.

In a second embodiment, the present invention provides a compound of formula (IB) or an N-oxide thereof, or a pharmaceutically acceptable salt thereof:

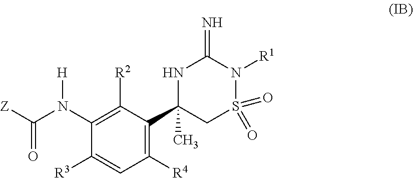

wherein

Z, R¹, R², R³ and R⁴ are as defined above.

In a first embodiment, Z represents aryl, which group may be optionally substituted by one or more substituents. In a second embodiment, Z represents heteroaryl, which group may be optionally substituted by one or more substituents.

Typically, Z represents phenyl, naphthyl, furyl, benzofuryl, dibenzofuryl, thienyl, benzothienyl, thieno[2,3-c]pyrazolyl, thieno[3,2-c]pyridinyl, dibenzothienyl, pyrrolyl, indolyl, pyrrolo[2,3-b]pyridinyl, pyrrolo[3,2-c]pyridinyl, pyrrolo[3,4-b]pyridinyl, pyrazolyl, pyrazolo[1,5-a]pyridinyl, pyrazolo[3,4-b]pyridinyl, pyrazolo[3,4-d]-pyrimidinyl, indazolyl, 4,5,6,7-tetrahydroindazolyl, oxazolyl, benzoxazolyl, isoxazolyl, thiazolyl, benzothiazolyl, isothiazolyl, imidazolyl, benzimidazolyl, imidazo[2,1-b]-thiazolyl, imidazo[1,2-a]pyridinyl, imidazo[1,5-a]pyridinyl, imidazo[4,5-b]pyridinyl, purinyl, imidazo[1,2-a]pyrimidinyl, imidazo[1,2-a]pyrazinyl, oxadiazolyl, thiadiazolyl, triazolyl, [1,2,4]triazolo[1,5-a]pyridinyl, [1,2,4]triazolo[1,5-a]pyrimidinyl, benzotriazolyl, tetrazolyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl or chromenyl, any of which groups may be optionally substituted by one or more substituents. Additionally, Z may represent [1,2,4]triazolo[4,3-a]pyridinyl or tetrazolo-[1,5-a]pyridinyl, either of which groups may be optionally substituted by one or more substituents.

Selected examples of Z include phenyl, naphthyl, furyl, benzofuryl, pyrrolyl, indolyl, pyrazolyl, imidazolyl, imidazo[1,2-a]pyridinyl, imidazo[1,5-a]pyridinyl, imidazo[1,2-a]pyrazinyl, oxadiazolyl, [1,2,4]triazolo[4,3-a]pyridinyl, tetrazolo[1,5-a]pyridinyl, pyridinyl, quinolinyl, naphthyridinyl, pyridazinyl, cinnolinyl, pyrimidinyl, pyrazinyl and quinoxalinyl, any of which groups may be optionally substituted by one or more substituents.

More particularly, Z represents phenyl, pyrazolyl, oxadiazolyl, pyridinyl, pyridazinyl, pyrimidinyl or pyrazinyl, any of which groups may be optionally substituted by one or more substituents.

Appositely, Z represents phenyl, pyridinyl, pyridazinyl, pyrimidinyl or pyrazinyl, any of which groups may be optionally substituted by one or more substituents.

Suitably, Z represents phenyl or pyridinyl, either of which groups may be optionally substituted by one or more substituents.

Typical examples of optional substituents on Z include one, two or three substituents independently selected from halogen, cyano, nitro, C₁₋₆ alkyl, difluoromethyl, trifluoromethyl, trifluoroethyl, hydroxy, hydroxy(C₁₋₆)alkyl, oxo, C₁₋₆ alkoxy, difluoro-methoxy, difluoroethoxy, trifluoromethoxy, trifluoroethoxy, phenoxy, methylenedioxy, difluoromethylenedioxy, C₁₋₆ alkylthio, C₁₋₆ alkylsulfinyl, C₁₋₆ alkylsulfonyl, amino, C₁₋₆ alkylamino, di(C₁₋₆)alkylamino, amino(C₁₋₆)alkyl, di(C₁₋₆)alkylamino(C₁₋₆)alkyl, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl, di(C₁₋₆)alkylamino-sulfonyl and di(C₁₋₆)alkylsulfoximino. Additional examples include C₂₋₆ alkynyl, cyclopropyl, morpholinyl, pyrazolyl, imidazolyl and (C₁₋₆)alkylimidazolyl.

Selected examples of optional substituents on Z include one, two or three substituents independently selected from halogen, cyano, C₁₋₆ alkyl, trifluoromethyl, C₂₋₆ alkynyl, cyclopropyl, C₁₋₆ alkoxy, difluoromethoxy, trifluoromethoxy, trifluoroethoxy, methylenedioxy, C₁₋₆ alkylsulfonyl, di(C₁₋₆)alkylamino, morpholinyl, pyrazolyl, imidazolyl and (C₁₋₆)alkylimidazolyl.

Apposite examples of optional substituents on Z include one, two or three substituents independently selected from halogen, cyano, C₁₋₆ alkyl and trifluoromethyl.

Suitable examples of optional substituents on Z include one, two or three substituents independently selected from halogen.

Typical examples of particular substituents on Z include one, two or three substituents independently selected from fluoro, chloro, bromo, cyano, nitro, methyl, ethyl, isopropyl, tert-butyl, difluoromethyl, trifluoromethyl, trifluoroethyl, hydroxy, hydroxymethyl, hydroxyethyl, hydroxyisopropyl, oxo, methoxy, isopropoxy, difluoro-methoxy, difluoroethoxy, trifluoromethoxy, trifluoroethoxy, phenoxy, methylenedioxy, difluoromethylenedioxy, methylthio, methylsulfinyl, methylsulfonyl, amino, methyl-amino, dimethylamino, aminomethyl, dimethylaminomethyl, acetylamino, methoxy-carbonylamino, methylsulfonylamino, formyl, acetyl, carboxy, methoxycarbonyl, ethoxy-carbonyl, aminocarbonyl, methylaminocarbonyl, dimethylaminocarbonyl, aminosulfonyl, methylaminosulfonyl, dimethylaminosulfonyl and dimethylsulfoximino. Additional examples include propynyl, cyclopropyl, morpholinyl, pyrazolyl, imidazolyl and methyl-imidazolyl.

Selected examples of particular substituents on Z include one, two or three substituents independently selected from fluoro, chloro, cyano, methyl, trifluoromethyl, propynyl, cyclopropyl, methoxy, difluoromethoxy, trifluoromethoxy, trifluoroethoxy, methylenedioxy, methylsulfonyl, dimethylamino, morpholinyl, pyrazolyl, imidazolyl and methylimidazolyl.

Apposite examples of particular substituents on Z include one, two or three substituents independently selected from fluoro, chloro, cyano, methyl and trifluoro-methyl.

Suitable examples of particular substituents on Z include one, two or three substituents independently selected from fluoro and chloro.

Selected values of Z include phenyl, fluorophenyl, chlorophenyl, cyanophenyl, methylphenyl, tert-butylphenyl, trifluoromethylphenyl, methoxyphenyl, isopropoxy-phenyl, difluoromethoxyphenyl, trifluoromethoxyphenyl, phenoxyphenyl, methylene-dioxyphenyl, difluoromethylenedioxyphenyl, methylsulfonylphenyl, methoxycarbonyl-phenyl, dimethylsulfoximinophenyl, difluorophenyl, (chloro)(fluoro)phenyl, (cyano)-(fluoro)phenyl, (fluoro)(methyl)phenyl, (fluoro)(methoxy)phenyl, (fluoro)(difluoro-methoxy)phenyl, (fluoro)(trifluoromethoxy)phenyl, (fluoro)(methylsulfonyl)phenyl, (chloro)(cyano)phenyl, (chloro)(methylsulfonyl)phenyl, (cyano)(trifluoromethyl)phenyl, (cyano)(methoxy)phenyl, (cyano)(difluoromethoxy)phenyl, dimethylphenyl, dimethoxy-phenyl, trifluorophenyl, naphthyl, (dimethyl)(phenyl)pyrazolyl, pyrazolo[1,5-a]pyridinyl, fluoropyrazolo[1,5-a]pyridinyl, methylpyrazolo[3,4-b]pyridinyl, methylindazolyl, imidazo[1,2-a]pyridinyl, imidazo[1,5-a]pyridinyl, methylimidazo[4,5-b]pyridinyl, [1,2,4]triazolo[1,5-a]pyridinyl, pyridinyl, fluoropyridinyl, chloropyridinyl, cyano-pyridinyl, methylpyridinyl, ethylpyridinyl, tert-butylpyridinyl, difluoromethylpyridinyl, trifluoromethylpyridinyl, trifluoroethylpyridinyl, methoxypyridinyl, difluoromethoxy-pyridinyl, trifluoromethoxypyridinyl, difluoroethoxypyridinyl, trifluoroethoxypyridinyl, dimethylaminopyridinyl, (fluoro)(methoxy)pyridinyl, (chloro)(methyl)pyridinyl, (chloro)-(trifluoromethyl)pyridinyl, (cyano)(methyl)pyridinyl, (cyano)(difluoromethyl)pyridinyl, (methyl)(trifluoromethyl)pyridinyl, (methyl)(oxo)pyridinyl, (methoxy)(methyl)pyridinyl, (difluoromethoxy)(methyl)pyridinyl, quinolinyl, cyanoquinolinyl, difluoromethoxy-quinolinyl, isoquinolinyl, methylisoquinolinyl, difluoromethoxyisoquinolinyl, methyl-pyridazinyl, trifluoroethoxypyridazinyl, methylpyrimidinyl, tert-butylpyrimidinyl, trifluoromethylpyrimidinyl, methylpyrazinyl, tert-butylpyrazinyl and difluoromethoxy-pyrazinyl. Additional values include (chloro)(methyl)phenyl, methylpyrazolyl, methyl-oxadiazolyl, pyridazinyl, pyrimidinyl and pyrazinyl. Additional values include propynyl-phenyl, pyrazolylphenyl, imidazolylphenyl, methylimidazolylphenyl, (fluoro)(trifluoro-methyl)phenyl, (chloro)(difluoromethoxy)phenyl, (methoxy)(methylsulfonyl)phenyl, (chloro)(difluoro)phenyl, methoxynaphthyl, cyanofuryl, fluorobenzofuryl, (cyano)-(methyl)pyrrolyl, methylindolyl, (methyl)(trifluoromethyl)pyrazolyl, methylimidazolyl, methylimidazo[1,2-a]pyridinyl, imidazo[1,2-a]pyrazinyl, [1,2,4]triazolo[4,3-a]pyridinyl, tetrazolo[1,5-a]pyridinyl, propynylpyridinyl, cyclopropylpyridinyl, morpholinylpyridinyl, difluoropyridinyl, (fluoro)(trifluoromethyl)pyridinyl, (methoxy)(trifluoromethyl)-pyridinyl, quinolinyl, naphthyridinyl, trifluoromethylpyridazinyl, cinnolinyl, chloro-pyrimidinyl, methoxypyrazinyl, dimethylaminopyrazinyl, quinoxalinyl and trifluoro-methylquinoxalinyl.

Particular values of Z include phenyl, fluorophenyl, chlorophenyl, cyanophenyl, trifluoromethylphenyl, propynylphenyl, methylenedioxyphenyl, methylsulfonylphenyl, pyrazolylphenyl, imidazolylphenyl, methylimidazolylphenyl, difluorophenyl, (chloro)-(fluoro)phenyl, (cyano)(fluoro)phenyl, (fluoro)(trifluoromethyl)phenyl, (fluoro)-(methoxy)phenyl, (fluoro)(trifluoromethoxy)phenyl, (chloro)(cyano)phenyl, (chloro)-(methyl)phenyl, (chloro)(difluoromethoxy)phenyl, (cyano)(trifluoromethyl)phenyl, (cyano)(methoxy)phenyl, (methoxy)(methylsulfonyl)phenyl, trifluorophenyl, (chloro)-(difluoro)phenyl, methoxynaphthyl, cyanofuryl, fluorobenzofuryl, (cyano)(methyl)-pyrrolyl, methylindolyl, methylpyrazolyl, (methyl)(trifluoromethyl)pyrazolyl, methyl-imidazolyl, methylimidazo[1,2-a]pyridinyl, imidazo[1,5-a]pyridinyl, imidazo[1,2-a]-pyrazinyl, methyloxadiazolyl, [1,2,4]triazolo[4,3-a]pyridinyl, tetrazolo[1,5-a]pyridinyl, pyridinyl, fluoropyridinyl, chloropyridinyl, cyanopyridinyl, methylpyridinyl, propynyl-pyridinyl, cyclopropylpyridinyl, trifluoromethylpyridinyl, methoxypyridinyl, trifluoro-methoxypyridinyl, trifluoroethoxypyridinyl, morpholinylpyridinyl, difluoropyridinyl, (fluoro)(trifluoromethyl)pyridinyl, (methyl)(trifluoromethyl)pyridinyl, (methoxy)-(trifluoromethyl)pyridinyl, quinolinyl, naphthyridinyl, pyridazinyl, methylpyridazinyl, trifluoromethylpyridazinyl, cinnolinyl, pyrimidinyl, chloropyrimidinyl, methyl-pyrimidinyl, pyrazinyl, methylpyrazinyl, methoxypyrazinyl, dimethylaminopyrazinyl, quinoxalinyl and trifluoromethylquinoxalinyl.

Apposite values of Z include phenyl, fluorophenyl, chlorophenyl, cyanophenyl, difluorophenyl, (chloro)(methyl)phenyl, methylpyrazolyl, methyloxadiazolyl, pyridinyl, fluoropyridinyl, chloropyridinyl, trifluoromethylpyridinyl, pyridazinyl, pyrimidinyl and pyrazinyl.

Typical values of Z include phenyl, fluorophenyl, difluorophenyl and chloro-pyridinyl.

Typically, R¹ represents C₃₋₇ cycloalkyl, aryl(C₁₋₆)alkyl, C₃₋₇ heterocycloalkyl, C₃₋₇ heterocycloalkyl(C₁₋₆)alkyl or heteroaryl(C₁₋₆)alkyl, any of which groups may be optionally substituted by one or more substituents.

Suitably, R¹ represents C₃₋₇ cycloalkyl, C₃₋₇ heterocycloalkyl or C₃₋₇ heterocycloalkyl(C₁₋₆)alkyl, any of which groups may be optionally substituted by one or more substituents.

More particularly, R¹ represents C₃₋₇ cycloalkyl or C₃₋₇ heterocycloalkyl, either of which groups may be optionally substituted by one or more substituents.

Appositely, R¹ represents C₃₋₇ heterocycloalkyl, which group may be optionally substituted by one or more substituents.

Suitable examples of R¹ include cyclobutyl, cyclohexyl, tetrahydrofuranyl, tetrahydropyranyl, oxetanylmethyl, tetrahydropyranylmethyl, 7-oxabicyclo[2.2.1]-heptanyl, 8-oxabicyclo[3.2.1]octanyl and 2-oxaspiro[3.3]heptanyl, any of which groups may be optionally substituted by one or more substituents.

Typical examples of R¹ include cyclobutyl, cyclohexyl, tetrahydrofuranyl, tetrahydropyranyl and tetrahydropyranylmethyl, any of which groups may be optionally substituted by one or more substituents.

Selected examples of R¹ include cyclohexyl and tetrahydropyranyl, either of which groups may be optionally substituted by one or more substituents.

A particular example of R¹ is tetrahydropyranyl, which group may be optionally substituted by one or more substituents.

Typical examples of optional substituents on R¹ include one, two or three substituents independently selected from halogen, cyano, nitro, C₁₋₆ alkyl, difluoromethyl, trifluoromethyl, hydroxy, hydroxy(C₁₋₆)alkyl, oxo, C₁₋₆ alkoxy, difluoromethoxy, trifluoromethoxy, C₁₋₆ alkylthio, C₁₋₆ alkylsulfinyl, C₁₋₆ alkylsulfonyl, amino, C₁₋₆ alkylamino, di(C₁₋₆)alkylamino, amino(C₁₋₆)alkyl, di(C₁₋₆)alkylamino(C₁₋₆)alkyl, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl and di(C₁₋₆)alkyl-aminosulfonyl.

Selected examples of optional substituents on R¹ include one, two or three substituents independently selected from halogen and C₁₋₆ alkyl.

Suitable examples of optional substituents on R¹ include one, two or three substituents independently selected from C₁₋₆ alkyl.

Typical examples of particular substituents on R¹ include one, two or three substituents independently selected from fluoro, chloro, bromo, cyano, nitro, methyl, ethyl, isopropyl, difluoromethyl, trifluoromethyl, hydroxy, hydroxymethyl, hydroxyethyl, hydroxyisopropyl, oxo, methoxy, difluoromethoxy, trifluoromethoxy, methylthio, methylsulfinyl, methylsulfonyl, amino, methylamino, dimethylamino, aminomethyl, dimethylaminomethyl, acetylamino, methoxycarbonylamino, methylsulfonylamino, formyl, acetyl, carboxy, methoxycarbonyl, ethoxycarbonyl, aminocarbonyl, methyl-aminocarbonyl, dimethylaminocarbonyl, aminosulfonyl, methylaminosulfonyl and dimethylaminosulfonyl.

Selected examples of particular substituents on R¹ include one, two or three substituents independently selected from fluoro and methyl.

Suitable examples of particular substituents on R¹ include one, two or three substituents independently selected from methyl.

Selected values of R¹ include difluorocyclohexyl, (difluoro)(methyl)cyclohexyl, tetrahydropyranyl, methyltetrahydropyranyl and dimethyltetrahydropyranyl.

Typical values of R¹ include tetrahydropyranyl and methyltetrahydropyranyl.

In a first embodiment, R¹ represents tetrahydropyranyl, especially tetrahydro-pyran-4-yl. In a second embodiment, R¹ represents methyltetrahydropyranyl, especially 2-methyltetrahydropyran-4-yl. In a third embodiment, R¹ represents dimethyltetrahydro-pyranyl, especially 2,6-dimethyltetrahydropyran-4-yl. In a fourth embodiment, R¹ represents difluorocyclohexyl, especially 4,4-difluorocyclohexyl. In a fifth embodiment, R¹ represents (difluoro)(methyl)cyclohexyl, especially 4,4-difluoro-3-methylcyclohexyl.

Generally, R², R³ and R⁴ independently represent hydrogen or halogen.

Generally, R² represents hydrogen or halogen.

In a first embodiment, R² represents hydrogen. In a second embodiment, R² represents halogen, especially fluoro or chloro. In one aspect of that embodiment, R² represents fluoro. In another aspect of that embodiment, R² represents chloro. In a third embodiment, R² represents trifluoromethyl.

Selected values of R² include hydrogen, fluoro and chloro.

Suitably, R² represents chloro.

Generally, R³ represents hydrogen or halogen, especially hydrogen.

In a first embodiment, R³ represents hydrogen. In a second embodiment, R³ represents halogen, especially fluoro or chloro. In one aspect of that embodiment, R³ represents fluoro. In another aspect of that embodiment, R³ represents chloro. In a third embodiment, R³ represents trifluoromethyl.

Selected values of R³ include hydrogen, fluoro and chloro.

Suitably, R³ represents hydrogen or fluoro.

Generally, R⁴ represents hydrogen or halogen, especially hydrogen.

In a first embodiment, R⁴ represents hydrogen. In a second embodiment, R⁴ represents halogen, especially fluoro or chloro. In one aspect of that embodiment, R⁴ represents fluoro. In another aspect of that embodiment, R⁴ represents chloro. In a third embodiment, R⁴ represents trifluoromethyl.

Suitably, R² represents hydrogen or halogen; R³ represents hydrogen or halogen; and R⁴ represents hydrogen.

Appositely, R² represents halogen; R³ represents hydrogen or halogen; and R⁴ represents hydrogen.

Generally, R² represents hydrogen or halogen; and R³ and R⁴ both represent hydrogen.

More particularly, R² represents halogen; and R³ and R⁴ both represent hydrogen.

One sub-class of compounds according to the invention is represented by the compounds of formula (IIA), and pharmaceutically acceptable salts thereof:

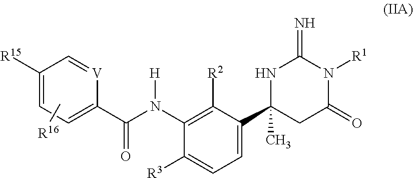

wherein

V represents N or CH;

R¹⁵ and R¹⁶ independently represent hydrogen, halogen, cyano, nitro, C₁₋₆ alkyl, difluoromethyl, trifluoromethyl, hydroxy, hydroxy(C₁₋₆)alkyl, C₁₋₆ alkoxy, difluoro-methoxy, trifluoromethoxy, phenoxy, C₁₋₆ alkylthio, C₁₋₆ alkylsulfinyl, C₁₋₆ alkylsulfonyl, amino, C₁₋₆ alkylamino, di(C₁₋₆)alkylamino, amino(C₁₋₆)alkyl, di(C₁₋₆)alkylamino(C₁₋₆)-alkyl, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkyl-aminocarbonyl, di(C₁₋₆)alkylaminocarbonyl, aminosulfonyl, C₁₋₆ alkylaminosulfonyl, di(C₁₋₆)alkylaminosulfonyl or di(C₁₋₆)alkylsulfoximino; and

R¹, R² and R³ are as defined above.

In a first embodiment, V represents N. In a second embodiment, V represents CH.

Appositely, R¹⁵ and R¹⁶ independently represent hydrogen, halogen, cyano, trifluoromethyl, C₁₋₆ alkoxy, trifluoromethoxy or C₁₋₆ alkylsulfonyl.

Suitably, R¹⁵ and R¹⁶ independently represent hydrogen, halogen, cyano or trifluoromethyl.

Typically, R¹⁵ and R¹⁶ independently represent hydrogen or halogen.

In general, R¹⁵ and R¹⁶ may independently represent hydrogen, fluoro, chloro, bromo, cyano, nitro, methyl, ethyl, isopropyl, tert-butyl, difluoromethyl, trifluoromethyl, hydroxy, hydroxymethyl, hydroxyethyl, hydroxyisopropyl, methoxy, isopropoxy, difluoromethoxy, trifluoromethoxy, phenoxy, methylthio, methylsulfinyl, methylsulfonyl, amino, methylamino, dimethylamino, aminomethyl, dimethylaminomethyl, acetylamino, methoxycarbonylamino, methylsulfonylamino, formyl, acetyl, carboxy, methoxycarbonyl, ethoxycarbonyl, aminocarbonyl, methylaminocarbonyl, dimethylaminocarbonyl, amino-sulfonyl, methylaminosulfonyl, dimethylaminosulfonyl or dimethylsulfoximino.

In principle, R¹⁵ and R¹⁶ may independently represent hydrogen, fluoro, chloro, cyano, trifluoromethyl, methoxy, trifluoromethoxy or methylsulfonyl.

More generally, R¹⁵ and R¹⁶ may independently represent hydrogen, fluoro, chloro, cyano or trifluoromethyl.

In particular, R¹⁵ and R¹⁶ may independently represent hydrogen, fluoro or chloro.

Typically, R¹⁵ represents hydrogen, halogen, cyano, C₁₋₆ alkyl, trifluoromethyl, C₁₋₆ alkoxy, difluoromethoxy, trifluoromethoxy, phenoxy, C₁₋₆ alkylsulfonyl, C₂₋₆ alkoxy-carbonyl or di(C₁₋₆)alkylsulfoximino.

More particularly, R¹⁵ represents hydrogen, halogen, cyano, trifluoromethyl or trifluoromethoxy.

Appositely, R¹⁵ represents hydrogen, halogen, cyano or trifluoromethyl.

Suitably, R¹⁵ represents hydrogen or halogen.

Particular values of R¹⁵ include hydrogen, fluoro, chloro, cyano, methyl, tert-butyl, trifluoromethyl, methoxy, isopropoxy, difluoromethoxy, trifluoromethoxy, phenoxy, methylsulfonyl, methoxycarbonyl and dimethylsulfoximino.

Selected values of R¹⁵ include hydrogen, fluoro, chloro, cyano, trifluoromethyl and trifluoromethoxy.

Apposite values of R¹⁵ include hydrogen, fluoro, chloro, cyano and trifluoro-methyl.

Specific values of R¹⁵ include hydrogen, fluoro and chloro.

In general, R¹⁶ represents hydrogen, halogen, cyano, trifluoromethyl, C₁₋₆ alkoxy, trifluoromethoxy or C₁₋₆ alkylsulfonyl.

Typically, R¹⁶ represents hydrogen, halogen, cyano, C₁₋₆ alkyl, trifluoromethyl or C₁₋₆ alkoxy.

Appositely, R¹⁶ represents hydrogen, halogen or cyano.

Suitably, R¹⁶ represents hydrogen or halogen.

Selected values of R¹⁶ include hydrogen, fluoro, chloro, cyano, trifluoromethyl, methoxy, trifluoromethoxy and methylsulfonyl.

Particular values of R¹⁶ include hydrogen, fluoro, chloro, cyano, methyl, trifluoromethyl and methoxy.

Apposite values of R¹⁶ include hydrogen, fluoro, chloro and cyano.

Illustrative values of R¹⁶ include hydrogen, fluoro and chloro.

Specific values of R¹⁶ include hydrogen and fluoro.

Another sub-class of compounds according to the invention is represented by the compounds of formula (IIB), and pharmaceutically acceptable salts thereof:

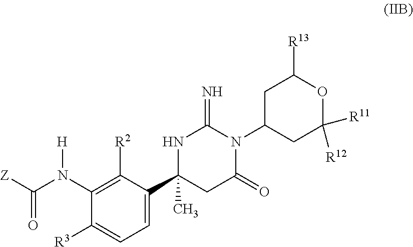

wherein

R¹¹ represents hydrogen or methyl;

R¹² represents hydrogen or methyl;

R¹³ represents hydrogen or methyl; and

Z, R² and R³ are as defined above.

In a first embodiment, R¹¹ represents hydrogen. In a second embodiment, R¹¹ represents methyl.

In a first embodiment, R¹² represents hydrogen. In a second embodiment, R¹² represents methyl.

In a first embodiment, R¹¹ and R¹² both represent hydrogen. In a second embodiment, R¹¹ represents hydrogen and R¹² represents methyl. In a third embodiment, R¹¹ and R¹² both represent methyl.

In a first embodiment, R¹³ represents hydrogen. In a second embodiment, R¹³ represents methyl.

Another sub-class of compounds according to the invention is represented by the compounds of formula (IIC), and pharmaceutically acceptable salts thereof:

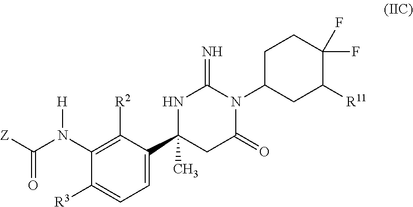

wherein

Z, R², R³ and R¹¹ are as defined above.

Specific novel compounds in accordance with the present invention include each of the compounds whose preparation is described in the accompanying Examples, and pharmaceutically acceptable salts thereof.

The present invention also provides a pharmaceutical composition which comprises a compound in accordance with the invention as described above, or a pharmaceutically acceptable salt thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Preparations for oral administration may be suitably formulated to give controlled release of the active compound.

For buccal administration, the compositions may take the form of tablets or lozenges formulated in conventional manner.

The compounds of formula (I) may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds of formula (I) may also be formulated as a depot preparation. Such long-acting formulations may be administered by implantation or by intramuscular injection.

For nasal administration or administration by inhalation, the compounds according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds of use in the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively, the compounds of use in the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds of use in the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration compounds may be formulated in an ointment such as petrolatum.

For rectal administration the compounds of use in the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

The quantity of a compound of use in the invention required for the prophylaxis or treatment of a particular condition will vary depending on the compound chosen and the condition of the patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

General methods for the preparation of the compounds of formula (I) as defined above are described in WO 2016/172255, WO 2011/044181, WO 2008/103351 and WO 2006/041404.

The compounds in accordance with the invention may be prepared by a process which comprises reacting a compound of formula Z—COCl with a compound of formula (III):

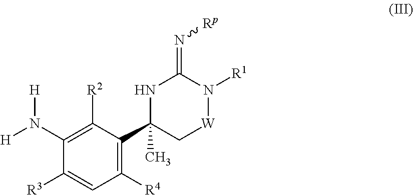

wherein W, Z, R¹, R², R³ and R⁴ are as defined above, and R^{p} represents hydrogen or an N-protecting group; followed, as necessary, by removal of the N-protecting group R^{p}.

The reaction between the compound of formula Z—COCl and compound (III) is conveniently accomplished at ambient temperature in the presence of pyridine.

Suitably, the N-protecting group R^{p} is tert-butoxycarbonyl (BOC).

Where the N-protecting group R^{p} is BOC, subsequent removal of the BOC group may suitably be accomplished by treatment with an acid, e.g. a mineral acid such as hydrochloric acid, or an organic acid such as trifluoroacetic acid. The reaction will typically be effected at ambient temperature in a suitable solvent, e.g. a chlorinated solvent such as dichloromethane, or a cyclic ether such as 1,4-dioxane.

In an alternative procedure, the compounds in accordance with the invention may be prepared by a two-step process which comprises: (i) treating a compound of formula Z—CO₂H with oxalyl chloride and N,N-dimethylformamide; and (ii) reacting the material thereby obtained with a compound of formula (III) as defined above; followed, as necessary, by removal of the N-protecting group R^{p}.

Step (i) is conveniently accomplished at ambient temperature in a suitable solvent, e.g. a chlorinated solvent such as dichloromethane.

Step (ii) is conveniently carried out in the presence of a base, e.g. an organic base such as triethylamine. The reaction is typically performed at a temperature in the region of 0° C. in a suitable solvent, e.g. a chlorinated solvent such as dichloromethane.

In another procedure, the compounds in accordance with the invention may be prepared by a process which comprises reacting a carboxylic acid of formula Z—CO₂H with a compound of formula (III) as defined above; in the presence of a coupling agent; followed, as necessary, by removal of the N-protecting group R^{p}.

Suitably, the coupling agent may be N,N,N′,N′-tetramethylchloroformamidinium hexafluorophosphate, in which case the reaction may generally be carried out in the presence of 1-methylimidazole. The reaction is conveniently performed at ambient temperature in a suitable solvent, e.g. a nitrile solvent such as acetonitrile.

Alternatively, the coupling agent may be 2,4,6-tripropyl-1,3,5,2,4,6-trioxa-triphosphorinane 2,4,6-trioxide, in which case the reaction may generally be carried out in the presence of a base which may suitably include organic amines, e.g. a trialkylamine such as N,N-diisopropylethylamine, or an aromatic base such as pyridine. The reaction is conveniently performed at ambient temperature in a suitable solvent, e.g. a chlorinated solvent such as dichloromethane.

Alternatively, the coupling agent may be 2-chloro-1-methylpyridinium iodide, in which case the reaction may generally be carried out in the presence of a base, e.g. a trialkylamine such as N,N-diisopropylethylamine. The reaction is conveniently performed at ambient temperature in a suitable solvent, e.g. a chlorinated solvent such as dichloromethane.

The intermediates of formula (III) above wherein W represents C(O) may be prepared by treating a compound of formula (IV):

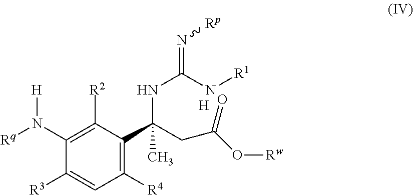

wherein R¹, R², R³, R⁴ and R^{p} are as defined above, R^{q} represents an N-protecting group, and R^{w} represents C₁₋₄ alkyl, especially methyl; with a base; followed by removal of the N-protecting group R^{q}.

Suitably, the base of use in the above reaction is a C₁₋₄ alkoxide salt, typically an alkali metal alkoxide such as potassium tert-butoxide. The reaction is conveniently accomplished at ambient temperature in a suitable solvent, e.g. a cyclic ether such as tetrahydrofuran.

Suitably, the N-protecting group R^{q} is benzyloxycarbonyl.

Where the N-protecting group R^{q} is benzyloxycarbonyl, subsequent removal of the benzyloxycarbonyl group may suitably be accomplished by catalytic hydrogenation. Typically, this will involve treatment with gaseous hydrogen in the presence of a hydrogenation catalyst such as palladium on charcoal.

The intermediates of formula (IV) above may be prepared by reacting a compound of formula (V) with a compound of formula (VI):

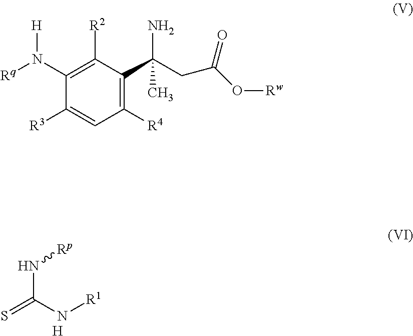

wherein R¹, R², R³, R⁴, R^{p}, R^{q} and R^{w} are as defined above.

Generally, the reaction between compounds (V) and (VI) is performed in the presence of a coupling agent. A suitable coupling agent is N-(3-dimethylaminopropyl)-N′-ethylcarbodiimide hydrochloride (EDC.HCl). Suitably, the reaction is performed in the presence of a base, typically an organic base such as N,N-diisopropylethylamine.

The reaction between compounds (V) and (VI) is conveniently accomplished at ambient temperature in a suitable solvent, e.g. a dipolar aprotic solvent such as N,N-dimethylformamide.

Under certain circumstances, the reaction between compounds (V) and (VI) will proceed directly to the corresponding compound of formula (III).

In an alternative procedure, the intermediates of formula (III) above may be prepared by treating a compound of formula (VII):

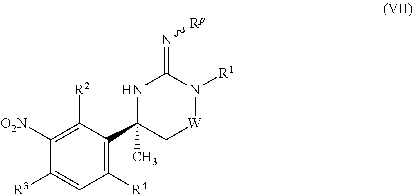

wherein W, R¹, R², R³, R⁴ and R^{p} are as defined above; with a reducing agent.

Suitably, the reducing agent of use in the above reaction may be a mixture of zinc and ammonium formate, in which case the reaction may conveniently be accomplished at ambient temperature in a suitable solvent, e.g. a C₁₋₄ alkanol such as methanol.

Alternatively, the reducing agent may be tin(II) chloride, in which case the reaction may conveniently be accomplished at an elevated temperature in a suitable solvent, e.g. a C₁₋₄ alkanol such as ethanol.

Alternatively, the compound of formula (VII) may be reduced by conventional catalytic hydrogenation, in which case the reaction may conveniently be accomplished by treating compound (VII) with hydrogen gas in the presence of a hydrogenation catalyst, e.g. palladium on charcoal. The reaction will typically be performed at ambient temperature in a suitable solvent, e.g. a C₁₋₄ alkanol such as methanol.

The intermediates of formula (VII) above wherein W represents C(O) may be prepared by treating a compound of formula (VIII):

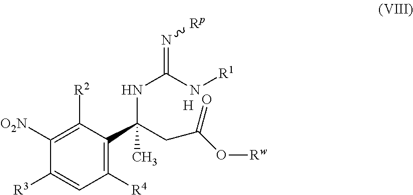

wherein R¹, R², R³, R⁴, R^{p} and R^{w} are as defined above; with a base; in a manner analogous to that described above for compound (IV).

The intermediates of formula (VIII) above may be prepared by reacting a compound of formula (VI) as defined above with a compound of formula (IX):

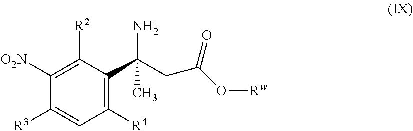

wherein R², R³, R⁴ and R^{w} are as defined above; employing conditions analogous to those described above for the reaction between compounds (V) and (VI).

Where they are not commercially available, the starting materials of formula (V), (VI) and (IX) may be prepared by methods analogous to those described in the accompanying Examples, or by standard methods well known from the art.

It will be understood that any compound of formula (I) initially obtained from any of the above processes may, where appropriate, subsequently be elaborated into a further compound of formula (I) by techniques known from the art.

Where a mixture of products is obtained from any of the processes described above for the preparation of compounds according to the invention, the desired product can be separated therefrom at an appropriate stage by conventional methods such as preparative HPLC; or column chromatography utilising, for example, silica and/or alumina in conjunction with an appropriate solvent system.

Where the above-described processes for the preparation of the compounds according to the invention give rise to mixtures of stereoisomers, these isomers may be separated by conventional techniques. In particular, where it is desired to obtain a particular enantiomer of a compound of formula (I) this may be produced from a corresponding mixture of enantiomers using any suitable conventional procedure for resolving enantiomers. Thus, for example, diastereomeric derivatives, e.g. salts, may be produced by reaction of a mixture of enantiomers of formula (I), e.g. a racemate, and an appropriate chiral compound, e.g. a chiral base. The diastereomers may then be separated by any convenient means, for example by crystallisation, and the desired enantiomer recovered, e.g. by treatment with an acid in the instance where the diastereomer is a salt. In another resolution process a racemate of formula (I) may be separated using chiral HPLC. Moreover, if desired, a particular enantiomer may be obtained by using an appropriate chiral intermediate in one of the processes described above. Alternatively, a particular enantiomer may be obtained by performing an enantiomer-specific enzymatic biotransformation, e.g. an ester hydrolysis using an esterase, and then purifying only the enantiomerically pure hydrolysed acid from the unreacted ester antipode. Chromatography, recrystallisation and other conventional separation procedures may also be used with intermediates or final products where it is desired to obtain a particular geometric isomer of the invention.

During any of the above synthetic sequences it may be necessary and/or desirable to protect sensitive or reactive groups on any of the molecules concerned. This may be achieved by means of conventional protecting groups, such as those described in Greene's Protective Groups in Organic Synthesis, ed. P. G. M. Wuts, John Wiley & Sons, 5^{th} edition, 2014. The protecting groups may be removed at any convenient subsequent stage utilising methods known from the art.

The following Examples illustrate the preparation of compounds according to the invention.

The compounds of the present invention are potent inhibitors of the growth and propagation of the Plasmodium falciparum: parasite in human blood. As such, they are active in a P. falciparum 3D7 asexual blood stage assay, exhibiting IC₅₀ values of 50 μM or less, generally of 20 μM or less, usually of 5 μM or less, typically of 1 μM or less, suitably of 500 nM or less, ideally of 100 nM or less, and preferably of 20 nM or less (the skilled person will appreciate that a lower IC₅₀ figure denotes a more active compound).

### Asexual Blood Stage Assay

The assay used to measure the effect of test compounds on a bloodstream stage of Plasmodium falciparum: 3D7 strain employs SYBR green as the readout. This is a dye that binds to double stranded deoxyribonucleic acid (DNA) with a resulting increase in fluorescence, allowing detection of P. falciparum DNA in infected erythrocytes, and thereby providing a measure of parasite growth and propagation.

P. falciparum Culture Maintenance

Erythrocytes (A+ blood) were prepared for both parasite culture and assay by washing 4 times with incomplete media (15.9 g RPMI 1640 (25 mM HEPES, L-glutamine), 1 g NaHCO₃, 2 g glucose, 400 μL gentacin (500 mg/mL), 2 mL hypoxanthine solution (13.6 g/L in 0.1M NaOH pH 7.3) in 1 litre of media). The cells were centrifuged at 1800 g for 5 minutes, before decanting the supernatant and re-suspending in fresh incomplete media. On the final wash, the cells were re-suspended in complete media (incomplete media with 5 g/L AlbumaxII), and centrifuged at 1800 g for 3 minutes. This cell sediment was treated as 100% haematocrit.

P. falciparum 3D7 was cultured in erythrocytes at 5% haematocrit in complete media at 37° C. (1% O₂, 3% CO₂, balance N₂). Cultures were split on a weekly basis to achieve a 1% parasitaemia in erythrocites at 5% haematocrit in fresh media. Culture media is replaced by fresh media every other day (2 times during the week).

### Assay Procedure

On day 1, test compounds were added to assay plates using Echo dispensing technology (1.5 fold dilution and 20 points titration). 50 nL of each compound dilution was added to 50 μL of culture (5% haematocrit, 0.5% parasitaemia) and incubated for 72 h at 37° C. (1% O₂, 3% CO₂, balance N₂). Final concentrations of test compounds ranged from 50,000 nM to 15 nM, in 0.5% DMSO.

On day 4, 10 μL SYBR green (Invitrogen S7563 supplied as 10,000× concentrate in DMSO) pre-diluted to 3× concentrate with Lysis buffer (20 mM Tris pH 7.9, 5% EDTA, 0.16% w/v, 1.6% TX100 v/v) was added to the cultures and incubated in the dark, overnight, at room temperature.

On day 5, fluorescent signal was measured using a BioTek plate reader (excitation 485 nm, emission 528 nm). All data were processed using IDBS ActivityBase. Raw data were converted into percent inhibition through linear regression by setting the high inhibition control (mefloquine) as 100% and the no inhibition control (DMSO) as 0%. Quality control criteria for passing plates were as follows: Z′>0.5, S:B>3, 30% CV_{(no inhibition control)}<15. The formula used to calculate Z′ is:

$$1 - \frac{3 ( σ p + σ n )}{( μ p - μ n )}$$

where μ denotes the mean; σ denotes the standard deviation; p denotes the positive control; and n denotes the negative control.

All EC₅₀ curve fitting was undertaken using the following bi-phasic two site dose response using XLfit model 300 (IDBS):

$$y = \frac{A}{1 + 1 {0}^{( C - log 10 ( Bx ) )}} + \frac{100 - A}{1 + 1 {0}^{( D - log 10 ( Bx ) )}}$$

where A=100 minus the top of the upper curve 1 and the bottom of lower curve; B=Hill slope; log(C)=IC₅₀ concentration at lower site; log(D)=IC₅₀ concentration at upper site; x=inhibitor concentration; and y=% inhibition.

When tested in the P. falciparum 3D7 asexual blood stage assay as described above, the compounds of the accompanying Examples were found to exhibit the following IC₅₀ values.

| Example | IC₅₀ (nM) |
|---|---|
| 1 | 227 |
| 2 | 1117 |
| 3 | 132 |
| 4 | 23 |
| 5 | 285 |
| 6 | 33 |
| 7 | 29 |
| 8 | 260 |
| 9 | 52 |
| 10 | 290 |
| 11 | 9 |
| 12 | 23 |
| 13 | 11 |
| 14 | 14 |
| 15 | 37 |
| 16 | 23 |
| 17 | 12 |
| 18 | 46 |
| 19 | 31 |
| 20 | 8 |
| 21 | 231 |
| 22 | 9 |
| 23 | 68 |
| 24 | 28 |
| 25 | 87 |
| 26 | 15 |
| 27 | 29 |
| 28 | 24 |
| 29 | 23 |
| 30 | 15 |
| 31 | 13 |
| 32 | 55 |
| 33 | 54 |
| 34 | 60 |
| 35 | 11 |
| 36 | 33 |
| 37 | 207 |
| 38 | 110 |
| 39 | 19 |
| 40 | 34 |
| 41 | 15 |
| 42 | 42 |
| 43 | 92 |
| 44 | 20 |
| 45 | 156 |
| 46 | 41 |
| 47 | 242 |
| 48 | 55 |
| 49 | 17 |
| 50 | 56 |
| 51 | 59 |
| 52 | 115 |
| 53 | 630 |
| 54 | 170 |
| 55 | 53 |
| 56 | 21 |
| 57 | 476 |
| 58 | 438 |
| 59 | 114 |
| 60 | 122 |
| 61 | 442 |
| 62 | 23 |
| 63 | 41 |
| 64 | 13 |
| 65 | 51 |
| 66 | 92 |
| 67 | 26 |
| 68 | 11 |
| 69 | 25 |
| 70 | 36 |
| 71 | 26 |
| 72 | 14 |
| 73 | 6 |
| 74 | 109 |
| 75 | 20 |
| 76 | 11 |
| 77 | 7 |
| 78 | 27 |
| 79 | 61 |
| 80 | 61 |
| 81 | 182 |
| 82 | 35 |
| 83 | 18 |
| 84 | 37 |
| 85 | 76 |
| 86 | 10 |
| 87 | 18 |
| 88 | 46 |
| 89 | 373 |
| 90 | 23 |
| 91 | 116 |
| 92 | 28 |
| 93 | 47 |
| 94 | 81 |
| 95 | 34 |
| 96 | 565 |
| 97 | 21 |
| 98 | 45 |
| 99 | 18 |
| 100 | 33 |
| 101 | 85 |
| 102 | 35 |
| 103 | 17 |
| 104 | 19 |
| 105 | 44 |
| 106 | 38 |
| 107 | 27 |
| 108 | 40 |
| 109 | 23 |
| 110 | 288 |
| 111 | 418 |
| 112 | 707 |
| 113 | 5 |
| 114 | 53 |
| 115 | 18 |
| 116 | 89 |
| 117 | 17 |

### EXAMPLES

### Abbreviations

DCM: dichloromethane EtOAc: ethyl acetate
DMSO: dimethyl sulfoxide THF: tetrahydrofuran
MeOH: methanol DMF: N,N-dimethylformamide
DIPEA: N,N-diisopropylethylamine TFA: trifluoroacetic acid
TFAA: trifluoroacetic anhydride EtOH: ethanol
DEA: diethylamine DMAP: 4-(dimethylamino)pyridine
DAST: (diethylamino)sulfur trifluoride LiHMDS: lithium bis(trimethylsilyl)amide
EDC.HCl: N-(3-dimethylaminopropyl)-N′-ethylcarbodiimide hydrochloride
TCFH: N,N,N′,N′-tetramethylchloroformamidinium hexafluorophosphate
T3P: 2,4,6-tripropyl-1,3,5,2,4,6-trioxatriphosphorinane-2,4,6-trioxide solution
Me₄tBuXPhos: 2-di-tert-butylphosphino-3,4,5,6-tetramethyl-2′,4′,6′-triisopropyl-1,1′-biphenyl
Pd₂(dba)₃: tris(dibenzylideneacetone)dipalladium(0)
h: hour M: mass
r.t.: room temperature RT: retention time

### DAD: Diode Array Detector

### HPLC: High Performance Liquid Chromatography

### LCMS: Liquid Chromatography Mass Spectrometry

### ESI: Electrospray Ionisation

### Nomenclature

Compounds were named in accordance with IUPAC guidelines with the aid of Biovia Draw version 16.1.

The asterisk (*)—for example, in compounds designated (2R*,4R*)—indicates compounds of known relative stereochemistry but unknown absolute stereochemistry.

### Materials

Commercially available Zn dust was activated by stirring with dilute 1N HCl, then washing with water, methanol and acetone, followed by drying under vacuum at 100-120° C. for 15 minutes.

### Analytical Conditions

### Method 1

### Column: Waters X Bridge C18, 2.1×30 mm, 2.5 μm

| | |
|---|---|
| Injection Volume | 5.0 μL |
| Flow Rate | 1.00 mL/minute |

### Detection:

MS—ESI+ m/z 150 to 800

UV—DAD 220-400 nm

Solvent A 5 mM ammonium formate in water+0.1% ammonia
Solvent B acetonitrile+5% Solvent A+0.1% ammonia
Gradient program:

5% B to 95% B in 4.0 minutes; hold until 5.00 minutes;

at 5.10 minutes concentration of B is 5%; hold up to 6.5 minutes

### Method 2

### Column: Waters UPLC X Bridge BEH (C18, 2.1×50 mm, 2.5 μm)

### Temperature: 45° C.

Injection volume: 1.0 μL
Flow rate: 1.00 mL/minute
Detection: Mass spectrometry—+/− detection in the same run

### PDA: 210 to 400 nm

Solvent A: 10 mM ammonium formate in water+0.1% formic acid
Solvent B: 95% acetonitrile+5% H₂O+0.1% formic acid

| Time | % A | % B |
|---|---|---|
| 0 | 95 | 55 |
| 0.10 | 95 | 5 |
| 2.10 | 5 | 95 |
| 2.35 | 5 | 95 |
| 2.80 | 95 | 5 |

### Method 3

### Column: Zorbax Extend C18 (50×4.6 mm, 5μ, 80 A)

Mobile phase: 50:50 [10 mM ammonium acetate in water]:acetonitrile to 5:95 [10 mM ammonium acetate in water]:acetonitrile gradient over 1.5 minutes, then continue elution to 4 minutes.
Flow rate: 1.2 mL/minute

### Intermediate 1

### N-[1-(2-Chloro-3-nitrophenyl)ethylidene]-(R)-2-methylpropane-2-sulfinamide

To a solution of 1-(2-chloro-3-nitrophenyl)ethanone (10.5 g, 5.1 mmol) and (R)-2-methyl-2-propanesulfinamide (11.2 g, 5.1 mmol) in dry THF (100 mL) was added titanium(IV) ethoxide (23.2 g, 10.5 mmol). The reaction mixture was heated at 75° C. for 12 h, then quenched with H₂O (500 mL), stirred at room temperature for 1 h and filtered through a pad of Celite. The aqueous layer was extracted with EtOAc (2×150 mL). The organic layer was separated and dried over anhydrous sodium sulfate, then concentrated in vacuo. The crude residue was purified by column chromatography (silica, 100-200 mesh, 30% EtOAc in hexanes) to afford the title compound (10.0 g, 63%) as a red liquid. LCMS (Method 1, ESI) 303.00 [MH]⁺, RT 3.02 minutes.

### Intermediate 2

### N-[1-(3-Amino-2-chlorophenyl)ethylidene]-2-(R)-methylpropane-2-sulfinamide

To a solution of Intermediate 1 (10.0 g, 33.2 mmol) in MeOH (100 mL) was added Raney Ni (10.0 g) at room temperature. The reaction mixture was stirred at room temperature for 6 h under hydrogen pressure, then filtered through a pad of Celite and washed with MeOH (150 mL). The filtrate was concentrated in vacuo to afford the title compound (8.80 g, 98%) as a colourless liquid, which was utilised without further purification. LCMS (Method 1, ESI) 273.00 [MH]⁺, RT 2.58 minutes.

### Intermediate 3

### Benzyl N-(3-{N—[(R)-tert-butylsulfinyl]-C-methylcarbonimidoyl}-2-chlorophenyl)-carbamate

To a solution of Intermediate 2 (10.0 g, 36.7 mmol) in THF (100 mL) were added DIPEA (32.5 mL, 183.0 mmol) and benzyl chloroformate (12.5 g, 73.5 mmol) at 0° C. The reaction mixture was stirred at room temperature for 16 h, then quenched with H₂O (500 mL) and extracted with EtOAc (3×250 mL). The organic layer was separated and dried over anhydrous sodium sulfate, then concentrated in vacuo. The crude residue was purified by column chromatography (silica, 100-200 mesh, 30% EtOAc in n-hexanes) to afford the title compound (12.5 g, 84%) as a yellow liquid. LCMS (Method 1, ESI) 407.00 [MH]⁺, RT 3.43 minutes.

### Intermediate 4

### Methyl (3S)-3-[3-(benzyloxycarbonylamino)-2-chlorophenyl]-3-{[(R)-tert-butylsulfinyl]-amino}butanoate

A suspension of CuCl (4.37 g, 44.2 mmol) and Zn (14.4 g, 221.0 mmol) in THF (90 mL) was heated at 50° C. for 30 minutes. Methyl bromoacetate (11.0 g, 66.0 mmol) was added dropwise at 80° C., then the reaction mixture was heated at 50° C. for 1 h. Intermediate 3 (9.00 g, 22.0 mmol) was added at 0° C. The reaction mixture was stirred at room temperature for 16 h, then filtered through a pad of Celite. The filtrate was washed with brine (300 mL). The organic layer was separated and dried over anhydrous sodium sulfate, then concentrated in vacuo. The crude residue was purified by column chromatography (silica, 100-200 mesh, 40% EtOAc in hexanes) to afford the title compound (7.50 g, 70%) as a yellow liquid. δ_{H} (400 MHz, DMSO-d₆) 9.09 (s, 1H), 7.54 (d, J 8.0 Hz, 1H), 7.29-7.43 (m, 7H), 5.39 (s, 1H), 5.14 (s, 2H), 3.47 (s, 3H), 3.31 (s, 2H), 1.86 (s, 3H) 1.13 (s, 9H). LCMS (Method 1, ESI) 481.00 [MH]⁺, RT 3.43 minutes.

### Intermediate 5

### Methyl (3S)-3-amino-3-[3-(benzyloxycarbonylamino)-2-chlorophenyl]butanoate

To a solution of Intermediate 4 (7.50 g, 15.6 mmol) in MeOH (80 mL) was added 4M HCl in 1,4-dioxane (15.6 mL, 62.5 mmol) at 0° C. The reaction mixture was stirred at room temperature for 6 h, then concentrated in vacuo. The residue was basified with saturated aqueous NaHCO₃ solution (200 mL) and extracted with EtOAc (2×250 mL). The organic layer was separated and dried over anhydrous sodium sulfate, then concentrated in vacuo, to afford the title compound (5.18 g, 90%) as a yellow liquid, which was utilised without further purification.

### Intermediate 6

### tert-Butyl N-(tetrahydropyran-4-ylcarbamothioyl)carbamate

To a solution of N,N′-bis-tert-butoxycarbonylthiourea (12.3 g, 44.5 mmol) in THF (100 mL) under nitrogen was added 60% NaH (5 g, 124.5 mmol) portionwise over a period of 10 minutes at 0° C. The mixture was stirred for 1 h, then TFAA (11.2 mL, 80.1 mmol) was added dropwise at 0° C. The mixture was stirred for 1 h, then a solution of tetrahydropyran-4-amine (4.5 g, 44.5 mmol) in THF (20 mL) was added. The reaction mixture was stirred at r.t. for 2 h, then quenched with ice-cold water and extracted with EtOAc (2×500 mL). The combined organic layers were dried over sodium sulfate, then the solvent was evaporated under reduced pressure. The crude residue was purified by column chromatography (silica gel, 100-200 mesh, 3% ethyl acetate/hexane) to afford the title compound (9.0 g, 77%) as a pale yellow solid. δ_{H} (400 MHz, CDCl₃) 9.68 (br s, 1H), 7.81 (br s, 1H), 4.46-4.44 (m, 1H), 3.95 (d, J 11.6 Hz, 2H), 3.52 (t, J 11.6 Hz, 2H), 2.07 (d, J 11.6 Hz, 2H), 1.61-1.53 (m, 2H), 1.47 (s, 9H).

### Intermediate 7

### Methyl (3S)-3-[3-(benzyloxycarbonylamino)-2-chlorophenyl]-3-{[N′-tert-butoxy-carbonyl-N-(tetrahydropyran-4-yl)carbamimidoyl]amino}butanoate

To a solution of Intermediate 5 (14 g, 33.9 mmol) and Intermediate 6 (9 g, 33.9 mmol) in DMF (100 mL) were added DIPEA (24 mL, 135.9 mmol) and EDC.HCl (13 g, 67.9 mmol) at 0° C. The reaction mixture was stirred at r.t. for 16 h, then diluted with ice-cold water and extracted with EtOAc (2×800 mL). The combined organic layers were washed with brine and dried over sodium sulfate, then the solvent was evaporated under reduced pressure. The crude residue was purified by column chromatography (silica gel, 100-200 mesh, 30% EtOAc/hexane) to afford the title compound (9 g, 44%) as an off-white solid. LCMS (Method 1, ESI) 603.85 [MH]⁺, RT 2.14 minutes.

### Intermediate 8

### tert-Butyl N-{(4S)-4-[3-(benzyloxycarbonylamino)-2-chlorophenyl]-4-methyl-6-oxo-1-(tetrahydropyran-4-yl)hexahydropyrimidin-2-ylidene}carbamate

To a solution of Intermediate 7 (9 g, 14.9 mmol) in THF (100 mL) was added potassium tert-butoxide in THF (1M, 29.84 mL, 29.8 mmol) under nitrogen at 0° C. over a period of 10 minutes. The reaction mixture was stirred at r.t. for 45 minutes, then quenched with aqueous ammonium chloride solution and extracted with EtOAc (2×800 mL). The combined organic layers were washed with brine and dried over sodium sulfate, then the solvent was evaporated under reduced pressure. The crude residue was purified by column chromatography (silica gel, 100-200 mesh, 30% EtOAc/hexane) to afford the title compound (7.5 g, 88%) as an off-white solid. LCMS (Method 1, ESI) 571.75 [MH]⁺, RT 2.21 minutes.

### Intermediate 9

### tert-Butyl N-[(4S)-4-(3-amino-2-chlorophenyl)-4-methyl-6-oxo-1-(tetrahydropyran-4-yl)-hexahydropyrimidin-2-ylidene]carbamate

To a solution of Intermediate 8 (8.0 g, 14.0 mmol) in methanol (100 mL) was added 10% Pd/C (800 mg). The reaction mixture was stirred under hydrogen balloon pressure at r.t. for 30 minutes, then filtered through celite and washed with methanol. The filtrate was concentrated under reduced pressure. The crude residue was purified by column chromatography (silica gel, 100-200 mesh, 30% EtOAc/hexane) to afford the title compound (5.5 g, 89%) as an off-white solid. δ_{H} (400 MHz, CDCl₃) 10.53 (br s, 1H), 6.99-7.05 (m, 1H), 6.75 (d, J 7.8 Hz, 1H), 6.68 (d, J 7.83 Hz, 1H), 4.74-4.85 (m, 1H), 4.20 (br s, 2H), 3.97 (dd, J 11.2, 4.4 Hz, 1H), 3.90 (dd, J 11.2, 4.40 Hz, 1H), 3.67 (dd, J 16.1, 1.5 Hz, 1H), 3.42-3.48 (m, 1H), 3.31-3.39 (m, 1H), 2.81 (d, J 16.63 Hz, 1H), 2.62-2.68 (m, 1H), 2.53-2.58 (m, 1H), 1.84 (s, 3H), 1.54 (s, 9H), 1.47-1.50 (m, 1H), 1.09-1.13 (m, 1H). LCMS (Method 1, ESI) 437.20 [MH]⁺, RT 2.08 minutes.

### Intermediate 10

### rac-(2S,4S)-2-Methyltetrahydropyran-4-amine

To a stirred solution of 2-methyltetrahydropyran-4-one (10.0 g, 87.6 mmol) in MeOH (100 mL) were added benzylamine (14.3 mL, 131.4 mmol) and acetic acid (0.25 mL, 4.38 mmol) under a nitrogen atmosphere. The mixture was stirred for 4 h at room temperature, then sodium cyanoborohydride (8.27 g, 131.4 mmol) was added at r.t. The reaction mixture was stirred for 16 h, then concentrated under reduced pressure. The crude residue was purified by column chromatography (100-200 mesh silica gel, eluting with 30-100% EtOAc/hexane). The resulting pale brown liquid was dissolved in MeOH (100 mL), and 10% Pd/C (10.0 g) was added in a Parr shaker vessel. The reaction mixture was stirred at r.t. for 16 h, then passed through a celite pad and washed with 10% MeOH in DCM. The filtrate was concentrated under reduced pressure to obtain the title compound (4.0 g, 71%) as a brown liquid. δ_{H} (400 MHz, DMSO-d₆) 3.81-3.77 (m, 1H), 3.32-3.23 (m, 2H), 2.71-2.63 (m, 1H), 2.32-1.86 (br s, 2H), 1.71-1.58 (m, 2H), 1.14-1.05 (m, 4H), 0.86 (q, J 12.3 Hz, 1H).

### Intermediate 11

### tert-Butyl N-{[rac-(2S,4S)-2-methyltetrahydropyran-4-yl]carbamothioyl}carbamate

Prepared from Intermediate 10 (3.16 g, 11.46 mmol) in accordance with the procedure described for Intermediate 6 to afford the title compound (2.1 g, 60%) as an off-white solid. δ_{H} (400 MHz, DMSO-d₆) 10.61 (s, 1H), 9.69 (d, J 7.5 Hz, 1H), 4.34-4.30 (m, 1H), 3.86 (dd, J 1.9, 10.8 Hz, 1H), 3.43-3.35 (m, 2H), 2.01 (d, J 10.6 Hz, 1H), 1.93 (d, J 12.2 Hz, 1H), 1.47 (s, 9H), 1.44-1.37 (m, 2H), 1.18-1.13 (m, 1H), 1.10 (d, J 6.12 Hz, 3H).

### Intermediate 12

### tert-Butyl (NE)-N-{(4S)-4-[3-(benzyloxycarbonylamino)-2-chlorophenyl]-4-methyl-1-[(2SR,4SR)-2-methyltetrahydropyran-4-yl]-6-oxohexahydropyrimidin-2-ylidene}-carbamate

Prepared from Intermediate 11 (2.0 g, 5.3 mmol) in accordance with the two-step procedure described for Intermediate 7 then Intermediate 8 to afford the title compound as an off-white solid. δ_{H} (400 MHz, DMSO-d₆) 10.51 (s, 1H), 9.25 (s, 1H), 7.58 (d, J 7.8 Hz, 1H), 7.40-7.32 (m, 6H), 7.17 (d, J 8.0 Hz, 1H), 5.13 (s, 2H), 4.68-4.62 (m, 1H), 3.82 (dd, J 2.8, 11.6, 1H), 3.74-3.71 (m, 1H), 3.58 (dd, J 2.8, 16.4 Hz, 1H), 3.29-3.17 (m, 3H), 2.35-2.21 (m, 1H), 1.75 (s, 3H), 1.44 (s, 9H), 1.07 (d, J 9.3 Hz, 2H), 1.05 (d, J 17.6 Hz, 2H).

### Intermediate 13

### tert-Butyl (NE)-N-{(4S)-4-(3-amino-2-chlorophenyl)-4-methyl-1-[(2S*,4S*)-2-methyl-tetrahydropyran-4-yl]-6-oxohexahydropyrimidin-2-ylidene}carbamate

Prepared from Intermediate 12 (1.5 g, 2.5 mmol) in accordance with the procedure described for Intermediate 9. The resulting racemic mixture was separated using chiral HPLC purification (chiral HPLC conditions: column: Chiralpak IC (250×20 mm) 5μ; mobile phase: hexane/EtOH/DEA: 80/20/0.1 (v/v/v); flow rate: 18 mL/minute; uv: 242 nm; runtime: 15 minutes) to afford the title compound (Peak 2 diastereomer 0.523 g) as an off-white solid.

Intermediate 13 (Peak 2): δ_{H} (400 MHz, DMSO-d₆) 10.47 (s, 1H), 7.00 (t, J 7.9 Hz, 1H), 6.78 (d, J 8.0 Hz, 1H), 6.46 (d, J 7.8 Hz, 1H), 5.52 (s, 2H), 4.69-4.63 (m, 1H), 3.75 (dd, J 4.5, 11.2 Hz, 1H), 3.50 (d, J 16.3 Hz, 1H), 3.23-3.18 (m, 2H), 3.11 (d, J 16.2 Hz, 1H), 2.33-2.22 (m, 1H), 2.11-2.02 (m, 1H), 1.73 (s, 3H), 1.44 (br s, 10H), 1.06 (d, J 6.0 Hz, 3H), 0.85 (d, J 7.0 Hz, 1H). LCMS (ESI, Method 3) m/e 451 [M+H]⁺, RT 1.56 minutes.

### Intermediate 14

### tert-Butyl N-[(4,4-difluorocyclohexyl)carbamothioyl]carbamate

Prepared from 4,4-difluorocyclohexanamine (4.09 g, 14.8 mmol) in accordance with the procedure described for Intermediate 6 to afford the title compound (1.9 g, 44%) as a yellow solid. δ_{H} (400 MHz, CDCl₃) 9.74 (d, J 3.91 Hz, 1H), 7.87 (br s, 1H), 4.30-4.44 (m, 1H), 2.05-2.24 (m, 4H), 1.84-2.01 (m, 2H), 1.62-1.81 (m, 2H), 1.50 (s, 9H).

### Intermediate 15

### Methyl (3S)-3-[3-(benzyloxycarbonylamino)-2-chlorophenyl]-3-{[(Z)—N′-tert-butoxy-carbonyl-N-(4,4-difluorocyclohexyl)carbamimidoyl]amino}butanoate

Prepared from Intermediate 14 (2.13 g, 5.16 mmol) in accordance with the procedure described for Intermediate 7 to afford the title compound (1 g, 66%) as a yellow solid. LCMS (Method 1, ESI) 637.25 [MH]⁺, RT 2.36 minutes.

### Intermediate 16

### tert-Butyl (NE)-N-{(4S)-4-[3-(benzyloxycarbonylamino)-2-chlorophenyl]-1-(4,4-difluorocyclohexyl)-4-methyl-6-oxohexahydropyrimidin-2-ylidene}carbamate

Prepared from Intermediate 15 (3.10 g, 4.25 mmol) in accordance with the procedure described for Intermediate 8 to afford the title compound (1.9 g, 58%) as an off-white solid. δ_{H} (400 MHz, CDCl₃) 10.60 (br s, 1H), 8.21 (d, J 7.83 Hz, 1H), 7.40-7.46 (m, 4H), 7.36-7.40 (m, 2H), 7.04 (dd, J 7.83, 1.47 Hz, 1H), 5.24 (s, 2H), 4.60-4.70 (m, 1H), 3.65 (d, J 16.63 Hz, 1H), 2.84 (d, J 16.14 Hz, 1H), 2.56-2.66 (m, 1H), 2.43-2.54 (m, 1H), 1.98-2.14 (m, 2H), 1.83 (s, 3H), 1.72-1.79 (m, 1H), 1.65-1.70 (m, 3H), 1.55 (s, 9H), 1.14 (d, J 12.72 Hz, 1H). LCMS (Method 1, ESI) 606 [MH]⁺, RT 2.35 minutes.

### Intermediate 17

### tert-Butyl (S,E)-[4-(3-amino-2-chlorophenyl)-1-(4,4-difluorocyclohexyl)-4-methyl-6-oxo-tetrahydropyrimidin-2(1H)-ylidene]carbamate

Prepared from Intermediate 16 (1.9 g, 3.03 mmol) in accordance with the procedure described for Intermediate 9 to afford the title compound (1.18 g, 82%) as an off-white solid. δ_{H} (400 MHz, CDCl₃) 10.55 (br s, 1H), 7.00-7.07 (m, 1H), 6.76 (dd, J 8.07, 1.22 Hz, 1H), 6.68 (dd, J 7.82, 1.47 Hz, 1H), 4.61-4.70 (m, 1H), 3.66 (d, J 16.63 Hz, 1H), 2.78-2.85 (m, 1H), 2.57-2.67 (m, 1H), 2.45-2.55 (m, 1H), 2.08-2.11 (m, 1H), 2.00-2.02 (m, 1H), 1.84 (s, 3H), 1.74-1.80 (m, 1H), 1.62-1.70 (m, 2H), 1.55 (s, 9H), 1.15-1.21 (m, 1H) (two exchangeable H signals of —NH₂ not observed). LCMS (Method 1, ESI) 471.20 [MH]⁺, RT 2.12 minutes.

### Intermediate 18

### N-{2-Chloro-3-[(4S)-1-(4,4-difluorocyclohexyl)-2-imino-4-methyl-6-oxohexahydro-pyrimidin-4-yl]phenyl}-3-cyanobenzamide Trifluoroacetic Acid Salt

Prepared from Intermediate 17 (0.15 g, 0.32 mmol) and 3-cyanobenzoyl chloride (0.11 g, 0.64 mmol) in accordance with General Method 1 to afford the title compound (0.09 g, 58%) as an off-white solid. LCMS (Method 1, ESI) 500.10 [MH]⁺, RT 2.25 minutes.

### Intermediate 19

### (2R*,6S*)-2,6-Dimethyltetrahydropyran-4-ol

To a stirred solution of 2,6-dimethyl-4H-pyran-4-one (20 g, 161.3 mmol) in ethanol (200 mL) was added 10% Pd—C (20 g). The reaction mixture was hydrogenated under a H₂ atmosphere (150 psi) at 50° C. for 16 h, then the catalyst was filtered off and the filtrate was evaporated, to afford the title compound (5.0 g, 24%). δ_{H} (400 MHz, CDCl₃) 3.81-3.74 (m, 1H), 3.47-3.40 (m, 2H), 2.02-1.89 (dd, 2H), 1.46-1.36 (m, 2H), 1.22-1.20 (m, 6H).

### Intermediate 20

### [(2R*,6S*)-2,6-Dimethyltetrahydropyran-4-yl] 4-nitrobenzoate

In an oven-dried round-bottomed flask Intermediate 19 (8 g, 61.5 mmol) was taken up in dry THF (50 mL) under inert conditions. The reaction mixture was cooled to 0° C., and 4-nitrobenzoic acid (20.5 g, 123.0 mmol) was added, followed by triphenyl-phosphine (32.24 g, 123.1 mmol). Diisopropyl azodicarboxylate (24.37 mL, 123.1 mmol) was added slowly, and the reaction mixture was stirred at room temperature for 18 h, then the solvent was evaporated under reduced pressure. The residue was purified by flash chromatography, eluting with ethyl acetate and hexane, to afford the title compound (4.5 g, 26%). δ_{H} (400 MHz, CDCl₃) 8.30-8.16 (m, 4H), 5.44 (s, 1H), 3.93-3.89 (m, 2H), 1.92-1.88 (m, 2H), 1.60-1.43 (m, 2H), 1.24-1.22 (m, 6H).

### Intermediate 21

### (2R*,6S*)-2,6-Dimethyltetrahydropyran-4-ol

To a stirred solution of Intermediate 20 (15 g, 53.8 mmol) in THF (300 mL) and H₂O (100 mL) was added LiOH.H₂O (11.2 g, 268.8 mmol). The resulting mixture was stirred at ambient temperature for 16 h, then the THF was removed under reduced pressure. The aqueous layer was acidified with 1N HCl, and the organic portion was extracted with EtOAc. The combined organic layers were dried (MgSO₄), filtered and concentrated under vacuum. The entire residue was purified via silica-gel column chromatography, eluting with EtOAc-hexane, to afford the title compound (4 g, 57%) as a colourless oil. δ_{H} (400 MHz, CDCl₃) 4.21-4.20 (m, 1H), 3.93-3.86 (m, 2H), 1.64-1.60 (m, 2H), 1.46-1.39 (m, 2H), 1.16-1.15 (m, 6H) (one exchangeable H signal of —OH not observed).

### Intermediate 22

### [(2R*,6S*)-2,6-Dimethyltetrahydropyran-4-yl] 4-methylbenzenesulfonate

To a stirred solution of Intermediate 21 (7 g, 53.8 mmol) in DCM (70 mL) were added pyridine (22.2 mL, 215.4 mmol), DMAP (657 mg, 5.4 mmol) and p-toluene-sulfonyl chloride (20.5 g, 107.7 mmol) at 0° C. The reaction mixture was stirred under a N₂ atmosphere at 23° C. for 24 h, then quenched with saturated aqueous NaHCO₃ solution. The organic layer was separated, washed with water and brine, then dried over anhydrous Na₂SO₄ and concentrated under reduced pressure. The resulting crude oily liquid was purified by silica gel column chromatography (100-200 mesh), eluting with EtOAc and hexane (1:9), to afford the title compound (7.8 g, 51%) as a light yellow liquid. δ_{H} (400 MHz, DMSO-d₆) 7.78 (d, 2H), 7.32 (d, 2H), 4.86 (s, 1H), 3.84-3.77 (m, 2H), 2.45 (s, 3H), 1.74 (d, 2H), 1.38-1.25 (m, 2H), 1.11 (s, 6H).

### Intermediate 23

### (2R*,6S*)-4-Azido-2,6-dimethyltetrahydropyran

To a stirred solution of Intermediate 22 (8 g, 28.2 mmol) in DMF (15 mL) was added sodium azide (5.5 g, 84.5 mmol). The reaction mixture was placed on an oil bath, pre-heated to 60° C., and stirred for 16 h, then allowed to cool and diluted with diethyl ether. The organic layer was washed with ice-cold water and separated, then dried over anhydrous Na₂SO₄ and concentrated under reduced pressure, to afford the title compound (3 g, 68%) as a yellow liquid. δ_{H} (400 MHz, CDCl₃) 3.48-3.42 (m, 3H), 1.92-1.88 (m, 2H), 1.23-1.16 (m, 8H).

### Intermediate 24

### (2R*,6S*)-2,6-Dimethyltetrahydropyran-4-amine

In an oven-dried round-bottomed flask Intermediate 23 (4 g, 25.5 mmol) was taken up in ethanol (15 mL) under an inert atmosphere, then 10% Pd/C (2 g) was added. The reaction mixture was stirred at room temperature in the presence of a H₂ balloon for 16 h, then passed through a Celite® pad and washed with 10% MeOH/DCM solution. The filtrate was concentrated under reduced pressure to afford the crude title compound (2.5 g, 76%) as a yellow liquid, which was utilised without further purification. δ_{H} (400 MHz, DMSO-d₆) 3.55-3.41 (m, 2H), 2.87-2.84 (m, 1H), 1.79-1.73 (m, 2H), 1.32-1.28 (m, 2H), 1.20-1.16 (m, 6H).

### Intermediate 25

### tert-Butyl N-{[(2S*,6R*)-2,6-dimethyltetrahydropyran-4-yl]carbamothioyl}carbamate

Prepared from Intermediate 24 (5.48 g, 19.84 mmol) in accordance with the procedure described for Intermediate 6 to afford the title compound (4 g, 56%). δ_{H} (400 MHz, DMSO-d₆) 10.62 (s, 1H), 9.68 (d, 1H), 4.35 (br s, 1H), 3.48-3.47 (m, 2H), 1.98 (d, 2H), 1.43 (s, 9H), 1.24-1.17 (m, 2H), 1.10 (d, 6H).

### Intermediate 26

### Methyl (3R)-3-[3-(benzyloxycarbonylamino)-2-chlorophenyl]-3-({(Z)—N′-tert-butoxy-carbonyl-N-[(2S*,6R*)-2,6-dimethyltetrahydropyran-4-yl]carbamimidoyl}amino)-butanoate

Prepared from Intermediate 25 (2.34 g, 8.1 mmol) in accordance with the procedure described for Intermediate 7 to afford the crude title compound (3.9 g, 91%) as a yellow solid, which was utilised without further purification.

### Intermediate 27

### tert-Butyl (NE)-N-{(4S)-4-[3-(benzyloxycarbonylamino)-2-chlorophenyl]-1-[(2S*,6R*)-2,6-dimethyltetrahydropyran-4-yl]-4-methyl-6-oxohexahydropyrimidin-2-ylidene}-carbamate

Prepared from Intermediate 26 (4.2 g, 6.65 mmol) in accordance with the procedure described for Intermediate 8 to afford the title compound (3.82 g, 96%). δ_{H} (400 MHz, DMSO-d₆) 10.52 (s, 1H), 9.24 (s, 1H), 7.58 (d, 1H), 7.39-7.33 (m, 6H), 7.16 (d, 1H), 5.14 (s, 2H), 4.68 (br s, 1H), 3.58-3.54 (m, 1H), 3.37 (br s, 1H), 3.28-3.24 (br s, 1H), 3.20-3.16 (m, 1H), 1.98 (m, 2H), 1.87 (d, 1H), 1.75 (s, 3H), 1.44 (s, 9H), 1.17 (m, 1H), 1.06 (m, 3H), 0.96 (m, 3H).

### Intermediate 28

### tert-Butyl (NE)-N-{(4S)-4-(3-amino-2-chlorophenyl)-1-[(2S*, 6R*)-2,6-dimethyl-tetrahydropyran-4-yl]-4-methyl-6-oxohexahydropyrimidin-2-ylidene}carbamate

Prepared from Intermediate 27 (3.5 g, 5.84 mmol) in accordance with the procedure described for Intermediate 9 to afford the title compound (1.65 g, 61%). δ_{H} (400 MHz, DMSO-d₆) 10.48 (s, 1H), 7.03-6.99 (m, 1H), 6.78 (d, 1H), 6.46 (d, 1H), 5.53 (s, 2H), 4.73-4.67 (m, 1H), 3.49 (d, 1H), 3.37-3.35 (br s, 1H), 3.30-3.27 (br s, 1H), 3.10 (d, 1H), 2.03-1.86 (m, 2H), 1.72 (s, 3H), 1.42 (s, 9H), 1.06 (d, 3H), 0.87 (d, 3H).

### Intermediate 29

### N-(2-Chloro-3-{(4S)-1-[(2S*,6R*)-2,6-dimethyltetrahydropyran-4-yl]-2-imino-4-methyl-6-oxohexahydropyrimidin-4-yl}phenyl)-3-cyanobenzamide Trifluoroacetic Acid Salt

Prepared from Intermediate 28 (0.20 g, 0.43 mmol) and 3-cyanobenzoic acid (0.14 g, 0.86 mmol) in accordance with General Method 2 to afford the title compound (0.12 g, 55%) as an off-white solid. δ_{H} (400 MHz, DMSO-d₆) 10.30 (br s, 1H), 8.42 (s, 1H), 8.29 (d, J 7.83 Hz, 1H), 8.09 (d, J 6.85 Hz, 1H), 7.77 (t, J 7.83 Hz, 1H), 7.49-7.53 (m, 2H), 7.37-7.44 (m, 1H), 4.19-4.40 (m, 1H), 2.91 (d, J 15.16 Hz, 1H), 1.96-2.14 (m, 2H), 1.62 (s, 3H), 1.51 (d, J 6.36 Hz, 1H), 1.07 (d, J 5.87 Hz, 3H), 0.99 (d, J 5.87 Hz, 3H), 0.88-0.90 (m, 1H) (three H signals merged in solvent peak; and two exchangeable H signals not observed). LCMS (Method 1, ESI) 494.20 [MH]⁺, RT 1.88 minutes.

### Intermediate 30

### tert-Butyl N-[(1RS,3RS)-3-methyl-4-oxocyclohexyl]carbamate

To a stirred solution of tert-butyl N-(4-oxocyclohexyl)carbamate (25 g, 117.4 mmol) in dry THF (250 mL) was added LiHMDS (1M in THF, 246.7 mL) at −78° C. The reaction mixture was stirred at −78° C. for 1 h, then triethylborane (1M in THF, 176.1 mL) was added. The reaction mixture was stirred at −78° C. for 1 h, then iodomethane (14.94 mL, 234.74 mmol) solution in THF (30 mL) was added at −78° C. The reaction mixture was stirred at room temperature for 12 h, then quenched with 1N aqueous NaOH solution. The mixture was stirred for 2 h, then diluted with H₂O and extracted with EtOAc. The organic layer was separated and washed with brine, then dried over anhydrous Na₂SO₄ and concentrated in vacuo. The crude residue was purified by column chromatography on silica gel (100-200 mesh size), eluting with 20% ethyl acetate in hexane. The resulting material was re-purified by combi-flash chromatography (15% EtOAc in hexanes) to afford the title compound (mixture with ˜15% of the opposite stereoisomer) (5 g, 19%) as an off-white solid. δ_{H} (400 MHz, DMSO-d₆) 6.82 (br s, 1H), 3.78-3.93 (m, 1H), 2.53-2.62 (m, 1H), 2.02-2.15 (m, 3H), 0.84-0.87 (m, 3H), 1.83-1.87 (m, 1H), 1.54-1.56 (m, 1H), 1.38 (s, 9H), 1.20-1.30 (m, 1H).

### Intermediate 31

### tert-Butyl N-[(1RS,3RS)-4,4-difluoro-3-methylcyclohexyl]carbamate

To a stirred solution of Intermediate 30 (20 g, 88.10 mmol) in DCM (200 mL) was added DAST (23.25 mL, 176.21 mmol) at 0° C. The reaction mixture was stirred at room temperature for 12 h, then diluted with ice-cold H₂O and extracted with DCM. The organic layer was separated and washed with brine, then dried over anhydrous Na₂SO₄ and concentrated in vacuo. The crude residue was purified by column chromatography (silica, 100-200 mesh, 10-20% EtOAc in hexanes) to afford the title compound (mixture with ˜15% of the opposite stereoisomer) (15 g, 68%) as a light yellow solid. δ_{H} (400 MHz, DMSO-d₆) 6.77-6.90 (m, 1H), 3.44-3.56 (m, 1H), 2.09-2.29 (m, 1H), 1.86-2.03 (m, 2H), 1.57-1.80 (m, 2H), 1.40-1.50 (br s, 1H), 1.38 (s, 9H), 0.87-0.97 (m, 3H) (one H signal merged in solvent peak).

### Intermediate 32

### (1RS,3RS)-4,4-Difluoro-3-methylcyclohexanamine hydrochloride

To a stirred solution of Intermediate 31 (15 g, 66.1 mmol) in MeOH (75 mL) was added 4M HCl in 1,4-dioxane (33 mL, 132.2 mmol) at 0° C. The reaction mixture was stirred at room temperature for 12 h, then concentrated in vacuo. The crude residue was washed with diethyl ether and pentane to afford the title compound (mixture with ˜10% of the opposite stereoisomer) (HCl salt) (9.7 g, 98%) as a light yellow solid. δ_{H} (400 MHz, DMSO-d₆) 8.31 (br s, 3H). 3.19-3.38 (m, 1H), 2.31-2.40 (m, 1H), 1.89-2.19 (m, 4H), 1.52-1.69 (m, 2H), 1.02-1.13 (m, 3H).

### Intermediate 33

### tert-Butyl N-{[(1RS,3RS)-4,4-difluoro-3-methylcyclohexyl]carbamothioyl}carbamate

Prepared from Intermediate 32 (746 mg, 2.70 mmol) in accordance with the procedure described for Intermediate 6 to afford the title compound (100 mg, 12%). δ_{H} (400 MHz, DMSO-d₆) 10.63-10.62 (br s, 1H), 9.69-9.68 (br s, 1H), 4.28-4.26 (m, 1H), 2.06-2.02 (m, 4H), 1.94-1.83 (m, 1H), 1.52-1.69 (m, 1H), 1.43 (s, 9H), 1.34-1.25 (m, 1H), 0.96 (d, 3H).

### Intermediate 34

### Methyl (3S)-3-[3-(benzyloxycarbonylamino)-2-chlorophenyl]-3-({(Z)—N′-tert-butoxy-carbonyl-N-[(1RS,3RS)-4,4-difluoro-3-methylcyclohexyl]carbamimidoyl}amino]-butanoate

Prepared from Intermediate 33 (1.25 g, 3.75 mmol) in accordance with the procedure described for Intermediate 7 to afford the crude title compound (2.50 g) as a thick brown oil, which was utilised without further purification. LCMS (Method 1, ESI) 651.28 [MH]⁺, RT 2.36 minutes.

### Intermediate 35

### tert-Butyl (NE)-N-{(4S)-4-[3-(benzyloxycarbonylamino)-2-chlorophenyl]-1-[(1RS,3RS)-4,4-difluoro-3-methylcyclohexyl]-4-methyl-6-oxohexahydropyrimidin-2-ylidene}-carbamate

Prepared from Intermediate 34 (2.50 g, 2.99 mmol) in accordance with the procedure described for Intermediate 8 to afford the title compound (2.40 g, 88%) as an off-white solid. LCMS (Method 1, ESI) 619.40 [MH]⁺, RT 1.68 minutes.

### Intermediate 36

### tert-Butyl (NE)-N-{(4S)-4-(3-amino-2-chlorophenyl)-1-[(1RS,3RS)-4,4-difluoro-3-methylcyclohexyl]-4-methyl-6-oxohexahydropyrimidin-2-ylidene}carbamate

Prepared from Intermediate 35 (2.40 g, 2.60 mmol) in accordance with the procedure described for Intermediate 9 to afford the title compound (0.84 g, 57%) as an off-white solid. LCMS (Method 1, ESI) 485.25 [MH]⁺, RT 2.35 minutes.

### Intermediate 37

### tert-Butyl (NE)-N-[(4S)-4-{2-chloro-3-[(3-cyanobenzoyl)amino]phenyl}-1-[(1S*,3S*)-4,4-difluoro-3-methylcyclohexyl]-4-methyl-6-oxohexahydropyrimidin-2-ylidene]-carbamate

### tert-Butyl (NE)-N-[(4S)-4-{2-chloro-3-[(3-cyanobenzoyl)amino]phenyl}-1-[(1R*,3R*)-4,4-difluoro-3-methylcyclohexyl]-4-methyl-6-oxohexahydropyrimidin-2-ylidene]-carbamate

To a solution of Intermediate 36 (0.12 g, 0.20 mmol) and 3-cyanobenzoyl chloride (0.06 g, 0.39 mmol) in dry DCM (6 mL) was added pyridine (0.05 mL, 0.58 mmol) at 0° C. The reaction mixture was stirred at room temperature for 2 h, then quenched with H₂O (50 mL) and extracted with DCM (2×50 mL). The organic layer was separated and concentrated in vacuo. The crude residue was purified by combi-flash chromatography (40% EtOAc in hexanes), and re-purified by chiral HPLC (Column: Phenomenex Cellulose-4, 250 mm×4.6 mm, 5 u; Mobile Phase A: n-hexane+0.1% isopropylamine; Mobile Phase B: EtOH; Flow rate: 1.00 mL/minute, isocratic: 10% B) to afford the title compounds (Peak 1, 0.025 g, 47%; and Peak 2, 0.023 g, 42%).

Peak 1: δ_{H} (400 MHz, DMSO-d₆) 10.55 (s, 1H), 8.41 (s, 1H) 10.33 (s, 1H), 8.28 (d, J 7.83 Hz, 1H), 8.09 (d, J 7.83 Hz, 1H), 7.77 (t, J 7.83 Hz, 1H), 7.59 (d, J 6.85 Hz, 1H), 7.45 (t, J 7.83 Hz, 1H), 7.30 (d, J 7.83 Hz, 1H), 4.59 (t, J 12.23 Hz, 1H), 3.62 (d, J 16.63 Hz, 1H), 3.23 (d, J 16.14 Hz, 2H), 1.45 (s, 9H), 1.03 (d, J 11.25 Hz, 1H), 0.93 (d, J 6.85 Hz, 3H), 2.20-2.33 (m, 3H), 1.80 (s, 3H) (two H signals merged in solvent peak). LCMS (Method 1, ESI) 614.25 [MH]⁺, RT 2.357 minutes.
Peak 2: δ_{H} (400 MHz, DMSO-d₆) 10.55 (s, 1H), 10.33 (s, 1H), 8.41 (s, 1H), 8.28 (d, J 7.83 Hz, 1H), 8.09 (d, J 7.83 Hz, 1H), 7.77 (t, J 7.83 Hz, 1H), 7.60 (d, J 7.83 Hz, 1H), 7.44-7.51 (m, 1H), 7.31 (d, J 7.83 Hz, 1H), 4.58-4.67 (m, 1H), 3.62 (d, J 16.14 Hz, 1H), 3.24 (d, J 16.14 Hz, 1H), 1.98-2.16 (m, 4H), 1.80 (s, 3H), 1.45 (s, 9H), 1.00-1.07 (m, 1H), 0.82 (d, J 6.36 Hz, 3H) (two H signals merged in solvent peak). LCMS (Method 1, ESI) 614.25 [MH]⁺, RT 2.35 minutes.

### Intermediate 38

### Methyl 5-cyclopropylnicotinate

To a solution of methyl 5-bromonicotinate (0.80 g, 3.70 mmol) in toluene (18 mL) and H₂O (2 mL) were added cyclopropylboronic acid (0.48 g, 5.55 mmol) and K₃PO₄ (2.36 g, 11.1 mmol) at room temperature. The reaction mixture was purged with argon for 10 minutes. Palladium(II) acetate (0.04 g, 0.19 mmol) and tricyclohexylphosphine (0.10 g, 0.37 mmol) were added, and the reaction mixture was again purged with argon for 10 minutes. The reaction mixture was heated at 100° C. for 2 h, then concentrated in vacuo. The residue was diluted with H₂O (400 mL) and extracted with EtOAc (2×400 mL). The organic layer was separated, washed with H₂O (150 mL) and brine (150 mL), then dried over anhydrous Na₂SO₄ and concentrated in vacuo. The crude residue was purified by column chromatography (silica, 100-200 mesh, 20% EtOAc in hexanes) to afford the title compound (0.503 g, 74%) as a yellow oil. δ_{H} (400 MHz, DMSO-d₆) 8.85 (s, 1H), 8.63 (s, 1H), 7.87 (s, 1H), 3.87 (s, 3H), 2.02-2.16 (m, 1H), 1.00-1.09 (m, 2H), 0.74-0.87 (m, 2H). LCMS (Method 1, ESI) 178.20 [MH]⁺, RT 1.81 minutes.

### Intermediate 39

### 5-Cyclopropylnicotinic Acid

To a solution of Intermediate 38 (0.50 g, 2.72 mmol) in THF (6 mL), MeOH (2 mL) and H₂O (2 mL) was added LiOH (0.26 g, 10.9 mmol) at 0° C. The reaction mixture was stirred at room temperature for 2 h, then concentrated in vacuo. The residue was diluted with H₂O (10 mL), then acidified with 1N HCl to pH 6 and extracted with EtOAc (2×30 mL). The organic layer was separated, dried over anhydrous Na₂SO₄ and concentrated in vacuo. The crude residue was purified by washing with diethyl ether (2 mL) and pentane (5 mL), then dried in vacuo, to afford the title compound (0.26 g, 58%) as an off-white solid. δ_{H} (400 MHz, DMSO-d₆) 13.35 (br s, 1H), 8.84 (s, 1H), 8.60 (s, 1H), 7.84 (s, 1H), 1.98-2.13 (m, 1H), 0.99-1.13 (m, 2H), 0.79-0.82 (m, 2H). LCMS (Method 1, ESI) 163.80 [MH]⁺, RT 1.10 minutes.

### Intermediate 40

### 3-Cyano-2-methoxybenzoic Acid

To a solution of 3-cyano-2-fluorobenzoic acid (0.80 g, 4.84 mmol) in MeOH (10 mL) was added sodium methoxide (30% in MeOH, 1.11 mL, 19.4 mmol) at r.t. The reaction mixture was heated at reflux for 2 h, then concentrated in vacuo. The residue was acidified with 1N HCl (50 mL) to pH 5 and filtered, then washed with H₂O (75 mL) and dried in vacuo, to afford the title compound (0.70 g, 81%) as an off-white solid, which was utilised without further purification. δ_{H} (400 MHz, DMSO-d₆) 13.50 (br s, 1H), 8.00 (t, J 7.09 Hz, 2H), 7.37 (t, J 7.83 Hz, 1H), 3.95 (s, 3H). LCMS (Method 1, ESI) 176.35 [MH]⁺, RT 1.33 minutes.

### Intermediate 41

### Methyl 5-(prop-1-ynyl)pyridine-3-carboxylate

To a solution of methyl 5-bromonicotinate (1, 2.00 g, 9.26 mmol) in DMSO (25 mL) were added DBU (4.15 mL, 27.8 mmol) and but-2-ynoic acid (2, 1.17 g, 13.9 mmol) at room temperature. The reaction mixture was purged with argon for 15 minutes, then bis(triphenylphosphine)palladium(II) dichloride (0.33 g, 0.46 mmol) and 1,2-bis-(diphenylphosphino)ethane (0.40 g, 0.93 mmol) were added. The reaction mixture was again purged with argon for 15 minutes and heated at 110° C. for 3 h, then quenched with H₂O (200 mL) and extracted with EtOAc (2×200 mL). The organic layer was separated and concentrated in vacuo. The crude residue was purified by combi-flash chromatography (50% EtOAc in hexanes) to afford the title compound (0.305 g, 19%) as an off-white solid. δ_{H} (400 MHz, DMSO-d₆) 8.98 (s, 1H), 8.81 (s, 1H), 8.19 (s, 1H), 2.10 (s, 3H), 3.88 (s, 3H).

### Intermediate 42

### 5-(Prop-1-ynyl)pyridine-3-carboxylic Acid

Prepared from Intermediate 41 (0.40 g, 2.28 mmol) in accordance with the procedure described for Intermediate 39 to afford the title compound (0.26 g, 70%) as an off-white solid. δ_{H} (400 MHz, DMSO-d₆) 13.61 (br s, 1H), 8.97 (s, 1H), 2.11 (s, 3H), 8.78 (s, 1H), 8.16 (s, 1H). LCMS (Method 1, ESI) 162.80 [MH]⁺, RT 1.39 minutes.

### Intermediate 43

### Methyl 3-(4-methylimidazol-1-yl)benzoate

An oven-dried vial was charged with Pd₂(dba)₃ (0.12 g, 0.14 mmol) and Me₄tBuXPhos (0.067 g, 0.14 mmol). The vial was sealed, then evacuated and backfilled with argon (three times in total). Anhydrous toluene (5 mL) was added, and the resulting premixed catalyst solution was stirred at 120° C. for 5 minutes. A second vial was charged with 4-methyl-1H-imidazole (1.37 g, 16.74 mmol), K₃PO₄ (5.91 g, 27.90 mmol) and methyl 3-bromobenzoate (3.0 g, 13.95 mmol), then the premixed catalyst solution was added by syringe to the second vial, followed by the addition of toluene (25 mL) and 1,4-dioxane (5 mL) (total 30 mL solvent). The reaction mixture was heated at 120° C. for 5 h, then cooled to room temperature and diluted with EtOAc. The organic layer was separated and washed with brine, then dried over anhydrous Na₂SO₄ and concentrated in vacuo. The crude residue was purified by flash chromatography to afford the title compound (2 g, 66.3%). δ_{H} (400 MHz, DMSO-d₆) 8.22 (s, 1H), 8.06 (s, 1H), 7.89 (br s, 2H), 7.65 (br t, 1H), 7.53 (s, 1H), 3.89 (s, 3H), 2.17 (s, 3H).

### Intermediate 44

### 3-(4-Methylimidazol-1-yl)benzoic Acid

To a stirred solution of Intermediate 43 (1.3 g, 4.90 mmol) in THF (36 mL) was added LiOH (1.03 g, 24.52 mmol) in water (12 mL). The resulting mixture was stirred at ambient temperature for 16 h, then the THF was removed under reduced pressure. The aqueous layer was diluted with more water and washed with diethyl ether, then the aqueous layer was acidified with aqueous citric acid and extracted with EtOAc. The organic layer was concentrated under reduced pressure. The crude residue was triturated with hexanes to afford the title compound (600 mg, 60.5%). δ_{H} (400 MHz, DMSO-d₆) 13.30 (br s, 1H), 8.19 (s, 1H), 8.03 (s, 1H), 7.87 (d, J 7.52 Hz, 1H), 7.82 (d, J 7.2 Hz, 1H), 7.60 (br t, 1H), 7.50 (s, 1H), 2.16 (s, 3H).

### Examples 1 TO 117

### General Method 1

To a solution of the appropriate aniline derivative in DCM was added pyridine (2 equivalents) at 0° C., followed by the addition of the appropriate acid chloride derivative (1.2 equivalents). The reaction mixture was stirred at room temperature for 2 h, then quenched with H₂O (20 mL) and extracted with EtOAc. The organic layer was separated, dried over anhydrous Na₂SO₄ and concentrated in vacuo. The crude residue was purified by column chromatography (silica, 100-200 mesh, 30% EtOAc in hexanes). The resulting material was redissolved in DCM, and TFA (20 equivalents) was added at 0° C. The reaction mixture was stirred at room temperature for 6 h, then concentrated under vacuum. The crude residue was purified by washing with diethyl ether (5 mL) and hexane (10 mL), then lyophilized with acetonitrile/H₂O (5 mL), to afford the title compound (TFA salt).

### General Method 2

To a solution of the appropriate carboxylic acid derivative in DCM was added DMF (1 drop), followed by the addition of oxalyl chloride (2.0 equivalents) at 0° C. The reaction mixture was stirred at room temperature for 3 h, then concentrated in vacuo. The residue was redissolved in DCM (3 mL), then triethylamine (6.0 equivalents) and the appropriate aniline derivative (1.05 equivalents) were added sequentially at 0° C. After completion, the reaction mixture was quenched with H₂O and extracted with DCM. The organic layer was separated, dried over anhydrous Na₂SO₄ and concentrated in vacuo. The crude residue was purified by column chromatography (silica, 100-200 mesh, 30% EtOAc in hexanes). The resulting material was redissolved in DCM and TFA (20 equivalents) was added at 0° C. The reaction mixture was stirred at room temperature for 6 h, then concentrated in vacuo. The crude residue was purified by washing with diethyl ether (5 mL) and hexane (10 mL), then lyophilized with acetonitrile/H₂O (5 mL), to afford the title compound (TFA salt).

### General Method 3

The appropriate TFA salt was dissolved in EtOAc and washed with saturated aqueous NaHCO₃ solution. The organic layer was separated, dried over anhydrous Na₂SO₄ and concentrated in vacuo. The crude residue was purified by preparative HPLC where required, then redissolved in dry DCM (8 mL). HCl in 1,4-dioxane (4M, 6 equivalents) was added at 0° C. The reaction mixture was stirred at room temperature for 30 minutes, then concentrated in vacuo and triturated with diethyl ether or DCM/n-pentane, to afford the title compound (HCl salt).

### General Method 4

To a solution of Intermediate 13 in acetonitrile were added the appropriate carboxylic acid derivative (1.5 equivalents) and 1-methylimidazole (2 equivalents), followed by the addition of TCFH (2 equivalents) at r.t. The reaction mixture was stirred at r.t. for 2-12 h, then quenched with H₂O and extracted with ethyl acetate. The organic layer was separated, washed with H₂O and brine, then dried over anhydrous Na₂SO₄ and concentrated in vacuo. The crude residue was purified by Combi flash column chromatography or HPLC. The resulting off-white solid was redissolved in DCM, and TFA (20 equivalents) was added at 0° C. The reaction mixture was stirred at r.t. for 6 h, then concentrated under vacuum. The crude residue was purified by washing with diethyl ether (5 mL) and hexane (10 mL), then lyophilized with acetonitrile/H₂O (5 mL), to afford the title compound (TFA salt).

### General Method 5

To a solution of Intermediate 13 in DCM were added the appropriate carboxylic acid derivative (1.5 equivalents), DIPEA (2 equivalents) and T3P (2 equivalents) at r.t. The reaction mixture was stirred at r.t. for 4-12 h, then quenched with H₂O and extracted with DCM. The organic layer was separated, washed with H₂O and brine, then dried over anhydrous Na₂SO₄ and concentrated in vacuo. The crude residue was purified by Combi flash column chromatography or HPLC. The resulting off-white solid was redissolved in DCM, and TFA (20 equivalents) was added at 0° C. The reaction mixture was stirred at r.t. for 6 h, then concentrated under vacuum. The crude residue was purified by washing with diethyl ether (5 mL) and hexane (10 mL), then lyophilized with acetonitrile/H₂O (5 mL), to afford the title compound (TFA salt).

### General Method 6

Intermediate 13 (20 mg, 0.044 mmol) was dissolved in DCM (1 mL). The appropriate carboxylic acid derivative (1.05 equivalents) and 2-chloro-1-methyl-pyridinium iodide (2.0 equivalents) were added, followed by DIPEA (3.0 equivalents). The reaction mixture was stirred at r.t. overnight. Where required, the reaction mixture was heated at 50° C. for 4 h, then stirred for an additional 16 h. The solvent was removed, then an acetonitrile/water solution (7:3) (990 μL) was added. The reaction mixture was purified by HPLC in basic mode. To the resulting material was added TFA/DCM (1:1) (1 mL). The reaction mixture was stirred for 1 h at r t, then the solvent was removed. The residue was purified by HPLC in acidic mode to afford the title compound (TFA salt).

### General Method 7

To a solution of the appropriate BOC-protected precursor (0.04 mmol) in DCM (2 mL) was added TFA (0.03 mL, 0.41 mmol) at 0° C. The reaction mixture was stirred at r.t. for 4 h, then concentrated in vacuo. The crude residue was purified by preparative HPLC (TFA method) to afford the title compound (TFA salt).

### Examples 1 to 117

Example 1 was prepared from Intermediate 9 and benzoyl chloride in accordance with General Method 1.

Example 2 was prepared from Intermediate 9 and 5-chloropyridine-2-carboxylic acid in accordance with General Method 2.

Example 3 was prepared from Intermediate 13 and 4-fluorobenzoyl chloride in accordance with General Method 1 followed by General Method 3.

Example 4 was prepared from Intermediate 13 and 2,4-difluorobenzoic acid in accordance with General Method 2 followed by General Method 3.

Example 5 was prepared from isonicotinic acid in accordance with General Method 4 followed by General Method 3.

Example 6 was prepared from pyridine-2-carboxylic acid in accordance with General Method 4 followed by General Method 3.

Example 7 was prepared from Intermediate 13 and nicotinic acid in accordance with General Method 2 followed by General Method 3.

Example 8 was prepared from Intermediate 13 and 1-methylpyrazole-3-carboxylic acid in accordance with General Method 2 followed by General Method 3.

Example 9 was prepared from Intermediate 13 and 4-chlorobenzoyl chloride in accordance with General Method 1 followed by General Method 3.

Example 10 was prepared from Intermediate 13 and 4-cyanobenzoic acid in accordance with General Method 2 followed by General Method 3.

Example 11 was prepared from Intermediate 13 and 3-chlorobenzoic acid in accordance with General Method 2 followed by General Method 3.

Example 12 was prepared from Intermediate 13 and 2-chlorobenzoic acid in accordance with General Method 2 followed by General Method 3.

Example 13 was prepared from 5-fluoropyridine-2-carboxylic acid in accordance with General Method 5 followed by General Method 3.

Example 14 was prepared from pyrazine-2-carboxylic acid in accordance with General Method 5 followed by General Method 3.

Example 15 was prepared from pyrimidine-2-carboxylic acid in accordance with General Method 5 followed by General Method 3.

Example 16 was prepared from pyrimidine-4-carboxylic acid in accordance with General Method 5 followed by General Method 3.

Example 17 was prepared from Intermediate 13 and 4-methyl-1,2,5-oxadiazole-3-carboxylic acid in accordance with General Method 2 followed by General Method 3.

Example 18 was prepared from 5-(trifluoromethyl)pyridine-2-carboxylic acid in accordance with General Method 5 followed by General Method 3.

Example 19 was prepared from pyridazine-3-carboxylic acid in accordance with General Method 5 followed by General Method 3.

Example 20 was prepared from Intermediate 13 and 3-cyanobenzoic acid in accordance with General Method 2 followed by General Method 3.

Example 21 was prepared from 6-(trifluoromethyl)pyridine-3-carboxylic acid in accordance with General Method 4 followed by General Method 3.

Example 22 was prepared from 3-chloro-5-methyl-benzoic acid in accordance with General Method 4 followed by General Method 3.

Example 23 was prepared from Intermediate 18 in accordance with General Method 3.

Example 24 was prepared from Intermediate 29 in accordance with General Method 3.

Example 25 was prepared from Intermediate 37 (Peak 1) in accordance with General Method 7.

Example 26 was prepared from Intermediate 37 (Peak 2) in accordance with General Method 7.

Example 27 was prepared from 1-methylindole-3-carboxylic acid in accordance with General Method 6.

Example 28 was prepared from 6-methylimidazo[1,2-a]pyridine-8-carboxylic acid in accordance with General Method 6.

Example 29 was prepared from tetrazolo[1,5-a]pyridine-8-carboxylic acid in accordance with General Method 6.

Example 30 was prepared from 2-methoxypyridine-3-carboxylic acid in accordance with General Method 6.

Example 31 was prepared from quinoxaline-2-carboxylic acid in accordance with General Method 6.

Example 32 was prepared from 2-methyl-5-(trifluoromethyl)pyrazole-3-carboxylic acid in accordance with General Method 6.

Example 33 was prepared from quinoline-3-carboxylic acid in accordance with General Method 6.

Example 34 was prepared from 5-methylpyrazine-2-carboxylic acid in accordance with General Method 6.

Example 35 was prepared from 2-fluorobenzoic acid in accordance with General Method 6.

Example 36 was prepared from 3,4-difluorobenzoic acid in accordance with General Method 6.

Example 37 was prepared from 4-(trifluoromethyl)benzoic acid in accordance with General Method 6.

Example 38 was prepared from pyrimidine-5-carboxylic acid in accordance with General Method 6.

Example 39 was prepared from 5-chloropyridine-3-carboxylic acid in accordance with General Method 6.

Example 40 was prepared from 5-fluoropyridine-3-carboxylic acid in accordance with General Method 6.

Example 41 was prepared from 3-(trifluoromethyl)benzoic acid in accordance with General Method 6.

Example 42 was prepared from 3,5-difluorobenzoic acid in accordance with General Method 6.

Example 43 was prepared from 1,6-naphthyridine-3-carboxylic acid in accordance with General Method 6.

Example 44 was prepared from 7-fluorobenzofuran-2-carboxylic acid in accordance with General Method 6.

Example 45 was prepared from 4-methoxypyridine-3-carboxylic acid in accordance with General Method 6.

Example 46 was prepared from cinnoline-3-carboxylic acid in accordance with General Method 6.

Example 47 was prepared from 5-(trifluoromethoxy)pyridine-2-carboxylic acid in accordance with General Method 6.

Example 48 was prepared from imidazo[1,2-a]pyrazine-8-carboxylic acid in accordance with General Method 6.

Example 49 was prepared from 3-(trifluoromethyl)pyridine-2-carboxylic acid in accordance with General Method 6.

Example 50 was prepared from 3-methoxypyridine-2-carboxylic acid in accordance with General Method 6.

Example 51 was prepared from 2-methylpyrazole-3-carboxylic acid in accordance with General Method 6.

Example 52 was prepared from Intermediate 13 and 1,5-naphthyridine-3-carboxylic acid in accordance with General Method 2.

Example 53 was prepared from Intermediate 13 and 6-cyclopropylpyridine-3-carboxylic acid in accordance with General Method 2 followed by General Method 3.

Example 54 was prepared from 6-methylpyridazine-3-carboxylic acid in accordance with General Method 5 followed by General Method 3.

Example 55 was prepared from 5-(trifluoromethyl)pyridazine-3-carboxylic acid in accordance with General Method 5 followed by General Method 3.

Example 56 was prepared from 4-(trifluoromethyl)pyridine-3-carboxylic acid in accordance with General Method 5 followed by General Method 3.

Example 57 was prepared from Intermediate 13 and 2-cyclopropylpyridine-3-carboxylic acid in accordance with General Method 2 followed by General Method 3.

Example 58 was prepared from Intermediate 13 and 6-(2,2,2-trifluoroethoxy)-pyridine-3-carboxylic acid in accordance with General Method 2 followed by General Method 3.

Example 59 was prepared from 6-(trifluoromethyl)pyridazine-3-carboxylic acid in accordance with General Method 5 followed by General Method 3.

Example 60 was prepared from Intermediate 13 and imidazo[1,5-a]pyridine-1-carboxylic acid in accordance with General Method 2 followed by General Method 3.

Example 61 was prepared from Intermediate 13 and 2-(trifluoromethyl)pyridine-3-carboxylic acid in accordance with General Method 2 followed by General Method 3.

Example 62 was prepared from Intermediate 13 and [1,2,4]triazolo[4,3-a]-pyridine-3-carboxylic acid in accordance with General Method 2 followed by General Method 3.

Example 63 was prepared from 5-chloropyridine-2-carboxylic acid in accordance with General Method 5 followed by General Method 3.

Example 64 was prepared from Intermediate 13 and 2,4,5-trifluorobenzoic acid in accordance with General Method 2 followed by General Method 3.

Example 65 was prepared from Intermediate 13 and imidazo[1,5-a]pyridine-3-carboxylic acid in accordance with General Method 2 followed by General Method 3.

Example 66 was prepared from 6-methylpyrazine-2-carboxylic acid in accordance with General Method 5 followed by General Method 3.

Example 67 was prepared from Intermediate 13 and 4-chloro-2-fluorobenzoic acid in accordance with General Method 2 followed by General Method 3.

Example 68 was prepared from 6-methoxypyrazine-2-carboxylic acid in accordance with General Method 5 followed by General Method 3.

Example 69 was prepared from Intermediate 13 and 5-chloro-2,4-difluorobenzoic acid in accordance with General Method 2 followed by General Method 3.

Example 70 was prepared from Intermediate 13 and 5-(trifluoromethyl)nicotinic acid in accordance with General Method 2 followed by General Method 3.

Example 71 was prepared from Intermediate 13 and 3-chloro-2,4-difluorobenzoic acid in accordance with General Method 2 followed by General Method 3.

Example 72 was prepared from 6-cyanopicolinic acid in accordance with General Method 5 followed by General Method 3.

Example 73 was prepared from Intermediate 13 and 3-cyano-2-fluorobenzoic acid in accordance with General Method 2 followed by General Method 3.

Example 74 was prepared from Intermediate 13 and 5-cyanonicotinic acid in accordance with General Method 2 followed by General Method 3.

Example 75 was prepared from 4-cyanopicolinic acid in accordance with General Method 5 followed by General Method 3.

Example 76 was prepared from Intermediate 13 and 5-cyano-2-fluorobenzoic acid in accordance with General Method 2 followed by General Method 3.

Example 77 was prepared from Intermediate 13 and 3-chloro-5-cyanobenzoic acid in accordance with General Method 2 followed by General Method 3.

Example 78 was prepared from 4-methylpyrimidine-2-carboxylic acid in accordance with General Method 5 followed by General Method 3.

Example 79 was prepared from 2-methoxy-1-naphthoic acid in accordance with General Method 6.

Example 80 was prepared from 4-fluoro-2-(trifluoromethyl)benzoic acid in accordance with General Method 6.

Example 81 was prepared from 2-(methylsulfonyl)benzoic acid in accordance with General Method 6.

Example 82 was prepared from 4-methylpyridine-3-carboxylic acid in accordance with General Method 6.

Example 83 was prepared from 2-fluoro-5-(trifluoromethyl)benzoic acid in accordance with General Method 6.

Example 84 was prepared from 5-chloro-2-(difluoromethoxy)benzoic acid in accordance with General Method 6.

Example 85 was prepared from 2-methoxy-5-(methylsulfonyl)benzoic acid in accordance with General Method 6.

Example 86 was prepared from 1,3-benzodioxole-4-carboxylic acid in accordance with General Method 6.

Example 87 was prepared from 5-fluoro-2-methoxybenzoic acid in accordance with General Method 6.

Example 88 was prepared from 3-(trifluoromethyl)quinoxaline-2-carboxylic acid in accordance with General Method 6.

Example 89 was prepared from 3-(trifluoromethyl)pyridine-4-carboxylic acid in accordance with General Method 6.

Example 90 was prepared from 3-(pyrazol-1-yl)benzoic acid in accordance with General Method 6.

Example 91 was prepared from 6-(dimethylamino)pyrazine-2-carboxylic acid in accordance with General Method 5 followed by General Method 3.

Example 92 was prepared from 5-chloropyrimidine-2-carboxylic acid in accordance with General Method 5 followed by General Method 3.

Example 93 was prepared from 4-(morpholin-4-yl)pyridine-2-carboxylic acid in accordance with General Method 5 followed by General Method 3.

Example 94 was prepared from 5-fluoro-3-(trifluoromethyl)pyridine-2-carboxylic acid in accordance with General Method 5 followed by General Method 3.

Example 95 was prepared from Intermediate 13 and 3-cyano-5-(trifluoromethyl)-benzoic acid in accordance with General Method 2 followed by General Method 3.

Example 96 was prepared from 4-fluoropyridine-3-carboxylic acid in accordance with General Method 5 followed by General Method 3.

Example 97 was prepared from 3-chloro-2-fluorobenzoic acid in accordance with General Method 5 followed by General Method 3.

Example 98 was prepared from Intermediate 13 and 2-chloro-5-cyanobenzoic acid in accordance with General Method 2 followed by General Method 3.

Example 99 was prepared from Intermediate 13 and 3-cyano-5-fluorobenzoic acid in accordance with General Method 2 followed by General Method 3.

Example 100 was prepared from 3,5-difluoropicolinic acid in accordance with General Method 5 followed by General Method 3.

Example 101 was prepared from Intermediate 13 and 2,4,6-trifluorobenzoic acid in accordance with General Method 2 followed by General Method 3.

Example 102 was prepared from Intermediate 13 and 4-fluoro-2-(trifluoro-methoxy)benzoic acid in accordance with General Method 2 followed by General Method 3.

Example 103 was prepared from Intermediate 13 and 3-methylimidazole-4-carboxylic acid in accordance with General Method 2 followed by General Method 3.

Example 104 was prepared from Intermediate 13 and Intermediate 39 in accordance with General Method 2 followed by General Method 3.

Example 105 was prepared from Intermediate 13 and 3-(1H-imidazol-1-yl)-benzoic acid in accordance with General Method 2 followed by General Method 3.

Example 106 was prepared from Intermediate 13 and 5-cyano-2-methoxybenzoic acid in accordance with General Method 2 followed by General Method 3.

Example 107 was prepared from Intermediate 13 and Intermediate 40 in accordance with General Method 2 followed by General Method 3.

Example 108 was prepared from Intermediate 13 and Intermediate 42 in accordance with General Method 2 followed by General Method 3.

Example 109 was prepared from Intermediate 13 and 3-(prop-1-ynyl)benzoic acid in accordance with General Method 2 followed by General Method 3.

Example 110 was prepared from Intermediate 13 and Intermediate 44 in accordance with General Method 2 followed by General Method 3.

Example 111 was prepared from Intermediate 13 and 2-methyl-4-(trifluoro-methyl)pyridine-3-carboxylic acid in accordance with General Method 2 followed by General Method 3.

Example 112 was prepared from Intermediate 13 and 1-oxidopyridin-1-ium-3-carboxylic acid in accordance with General Method 2 followed by General Method 3.

Example 113 was prepared from Intermediate 13 and 4-cyano-1-methylpyrrole-2-carboxylic acid in accordance with General Method 2 followed by General Method 3.

Example 114 was prepared from Intermediate 13 and 2-methoxy-4-(trifluoro-methyl)pyridine-3-carboxylic acid in accordance with General Method 2 followed by General Method 3.

Example 115 was prepared from 5-cyanofuran-2-carboxylic acid in accordance with General Method 5.

Example 116 was prepared from 3-cyano-4-fluorobenzoic acid in accordance with Genera/Method 6.

Example 117 was prepared from 3-cyano-4-chlorobenzoic acid in accordance with Genera/Method 6.

| | | | LCMS (Method 2) | |
|---|---|---|---|---|
| Ex. | Product | Structure | RT (min) | [MH]⁺ |
| 1 | N-{2-Chloro-3-[(4S)-2-imino-4- methyl-6-oxo-1-(tetrahydropyran- 4-yl)hexahydropyrimidin-4-yl]- phenyl]benzamide trifluoroacetic acid salt | 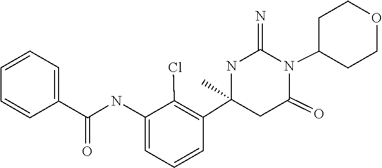 | 0.66 | 441 |
| 2 | 5-Chloro-N-{2-chloro-3-[(4S)-2- imino-4-methyl-6-oxo-1- (tetrahydropyran-4-yl)hexahydro- pyrimidin-4-yl] phenyl}pyridine-2- carboxamide trifluoroacetic acid salt | 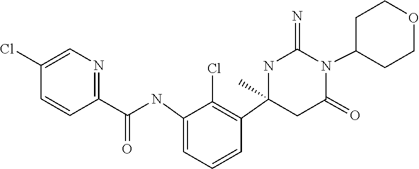 | 0.82 | 476 |
| 3 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 4-fluorobenzamide hydrochloride | 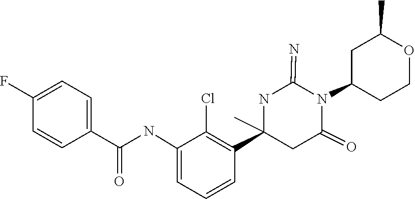 | 0.73 | 473 |
| 4 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 2,4-difluorobenzamide hydrochloride | 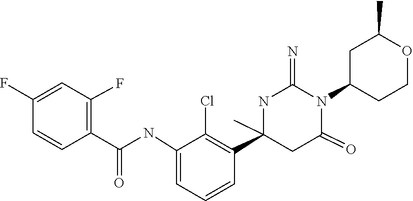 | 0.79 | 491 |
| 5 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- pyridine-4-carboxamide hydrochloride | 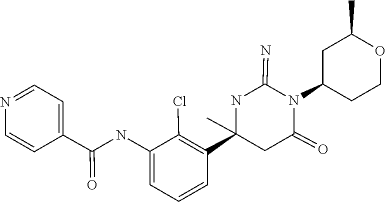 | 0.53 | 456 |
| 6 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- pyridine-2-carboxamide hydrochloride | 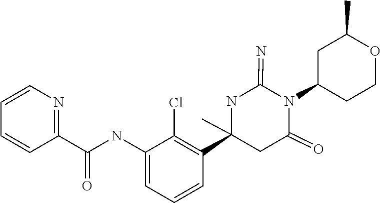 | 0.72 | 456 |
| 7 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- pyridine-3-carboxamide hydrochloride | 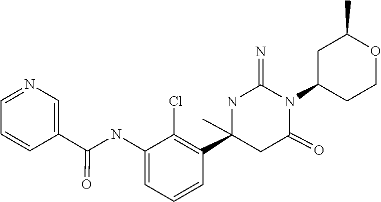 | 0.50 | 456 |
| 8 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 1-methylpyrazole-3-carboxamide hydrochloride | 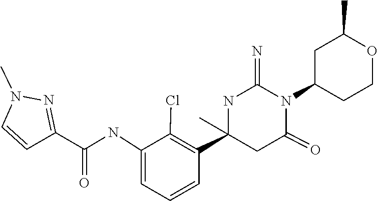 | 0.60 | 459 |
| 9 | 4-Chloro-N-(2-chloro-3-{(4S)-2- imino-4-methyl-1-[(2R*,4R*)-2- methyltetrahydropyran-4-yl]-6-oxo- hexahydropyrimidin-4-yl}phenyl)- benzamide hydrochloride | 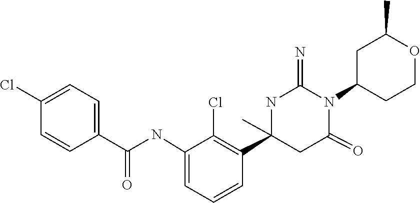 | 0.78 | 489 |
| 10 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 4-cyanobenzamide hydrochloride | 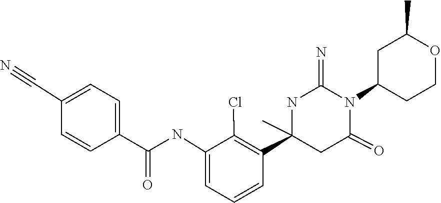 | 0.68 | 480 |
| 11 | 3-Chloro-N-(2-chloro-3-{(4S)-2- imino-4-methyl-1-[(2R*,4R*)-2- methyltetrahydropyran-4-yl]-6-oxo- hexahydropyrimidin-4-yl]phenyl)- benzamide hydrochloride | 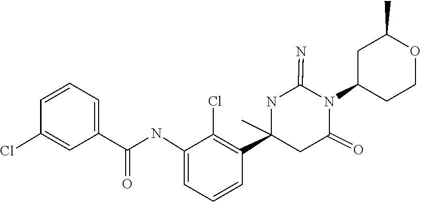 | 0.81 | 489 |
| 12 | 2-Chloro-N-(2-chloro-3-{(4S)-2- imino-4-methyl-1-[(2R*,4R*)-2- methyltetrahydropyran-4-yl]-6-oxo- hexahydropyrimidin-4-yl}phenyl)- benzamide hydrochloride | 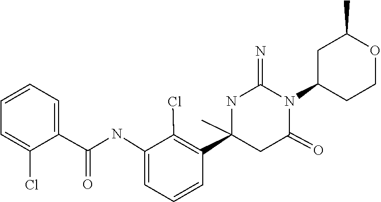 | 0.73 | 489 |
| 13 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 5-fluoropyridine-2-carboxamide hydrochloride | 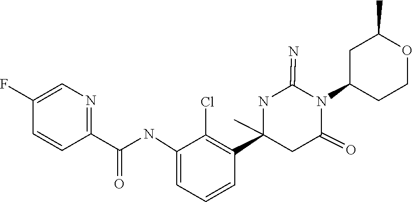 | 0.77 | 474 |
| 14 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- pyrazine-2-carboxamide hydrochloride | 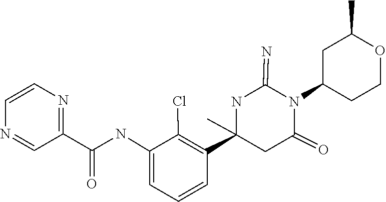 | 0.64 | 457 |
| 15 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- pyrimidine-2-carboxamide hydrochloride | 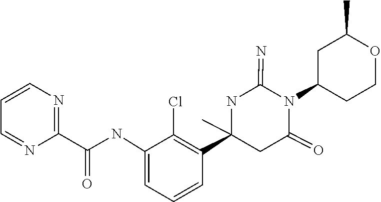 | 0.56 | 457 |
| 16 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- pyrimidine-4-carboxamide hydrochloride | 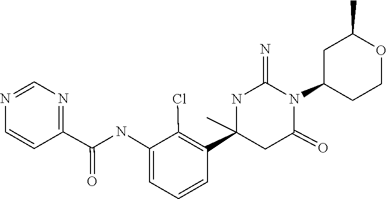 | 0.63 | 457 |
| 17 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 4-methyl-1,2,5-oxadiazole-3- carboxamide hydrochloride | 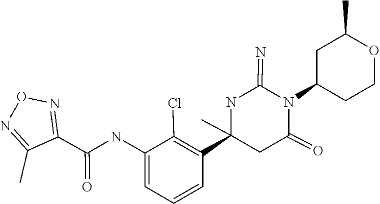 | 0.71 | 461 |
| 18 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 5-(trifluoromethyl)pyridine-2- carboxamide hydrochloride | 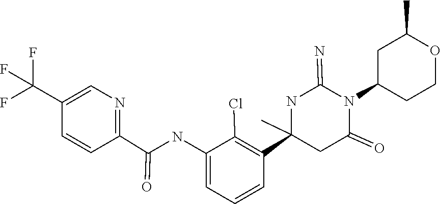 | 0.91 | 524 |
| 19 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- pyridazine-3-carboxamide hydrochloride | 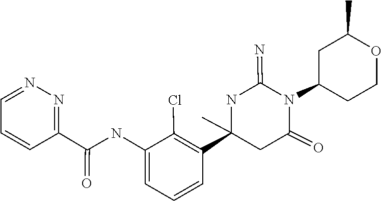 | 0.62 | 457 |
| 20 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 3-cyanobenzamide hydrochloride | 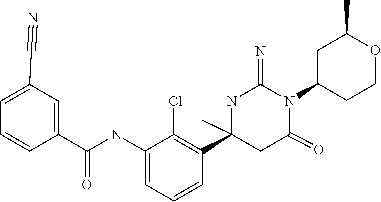 | 0.68 | 480 |
| 21 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 6-(trifluoromethyl)pyridine-3- carboxamide hydrochloride | 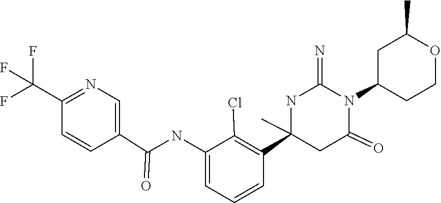 | 0.74 | 524 |
| 22 | 3-Chloro-N-(2-chloro-3-{(4S)-2- imino-4-methyl-1-[(2R*,4R*)-2- methyltetrahydropyran-4-yl]-6-oxo- hexahydropyrimidin-4-yl}phenyl)- 5-methylbenzamide hydrochloride | 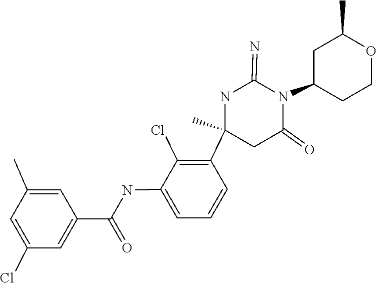 | 0.89 | 503 |
| 23 | N-{2-Chloro-3-[(4S)-1-(4,4- difluorocyclohexyl)-2-imino-4- methyl-6-oxohexahydropyrimidin- 4-yl]phenyl}-3-cyanobenzamide hydrochloride | 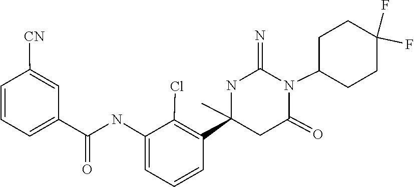 | 2.47 | 500 |
| 24 | N-(2-Chloro-3-{(4S)-1-[(2S*,6R*)- 2,6-dimethyltetrahydropyran-4-yl]- 2-imino-4-methyl-6-oxohexahydro- pyrimidin-4-yl]phenyl)-3-cyano- benzamide hydrochloride | 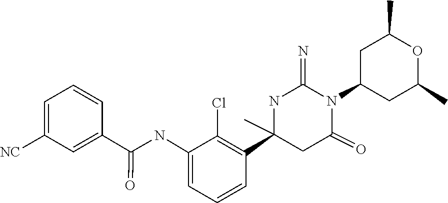 | 2.96 | 494 |
| 25 | N-(2-Chloro-3-{(4S)-1-[(1R*,3R*)- 4,4-difluoro-3-methylcyclohexyl]- 2-imino-4-methyl-6-oxohexahydro- pyrimidin-4-yl]phenyl)-3-cyano- benzamide trifluoroacetic acid salt | 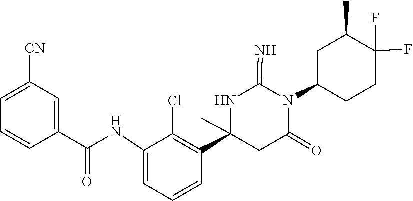 [SMILES: C[C@@H]1C[C@H](N2C(=N)N[C@](C)(c3cccc(NC(=O)c4cccc(C#N)c4)c3Cl)CC2=O)CCC1(F)F] | 2.62 | 514 |
| 26 | N-(2-Chloro-3-{(4S)-1-[(1S*,3S*)- 4,4-difluoro-3-methylcyclohexyl]- 2-imino-4-methyl-6-oxohexahydro- pyrimidin-4-yl]phenyl)-3-cyano- benzamide trifluoroacetic acid salt | 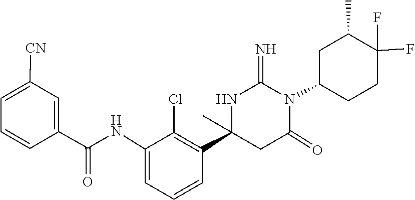 [SMILES: C[C@H]1C[C@@H](N2C(=N)N[C@](C)(c3cccc(NC(=O)c4cccc(C#N)c4)c3Cl)CC2=O)CCC1(F)F] | 2.61 | 514 |
| 27 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 1-methylindole-3-carboxamide trifluoroacetic acid salt | 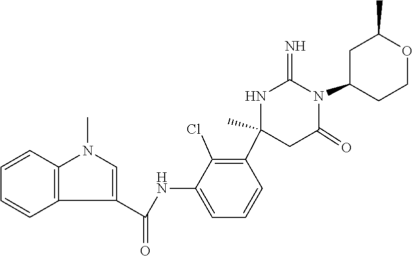 [SMILES: C[C@@H]1C[C@H](N2C(=N)N[C@](C)(c3cccc(NC(=O)c4cn(C)c5ccccc45)c3Cl)CC2=O)CCO1] | 0.81 | 508.1 |
| 28 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 6-methylimidazo[1,2-a]pyridine-8- carboxamide trifluoroacetic acid salt | 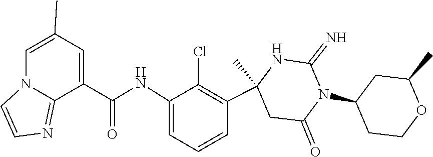 [SMILES: Cc1cc(C(=O)Nc2cccc([C@]3(C)CC(=O)N([C@@H]4CCO[C@H](C)C4)C(=N)N3)c2Cl)c2nccn2c1] | 0.71 | 509.4 |
| 29 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- tetrazolo[1,5-a] pyridine-8- carboxamide trifluoroacetic acid salt | 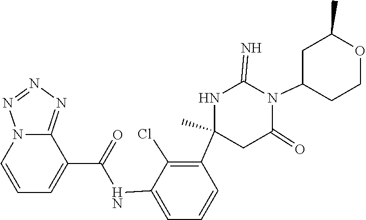 [SMILES: C[C@@H]1CC(N2C(=N)N[C@](C)(c3cccc(NC(=O)c4cccn5nnnc45)c3Cl)CC2=O)CCO1] | 0.67 | 497.4 |
| 30 | N-(2-Chloro-3-{(4S)-2-imino-4- methyl-1-[(2R*,4R*)-2-methyl- etrahydropyran-4-yl]-6-oxo- hexahydropyrimidin-4-yl]phenyl)- 2-methoxypyridine-3-carboxamide trifluoroacetic acid salt | 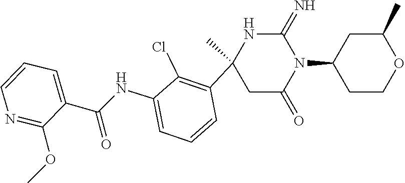 | 0.78 | 486.4 |
| 31 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- quinoxaline-2-carboxamide trifluoroacetic acid salt | 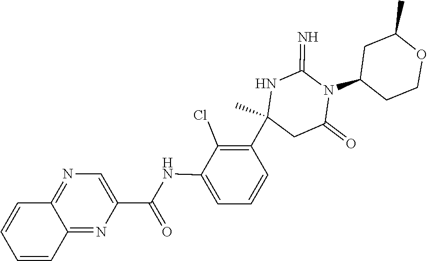 [SMILES: C[C@@H]1C[C@H](N2C(=N)N[C@](C)(c3cccc(NC(=O)c4cnc5ccccc5n4)c3Cl)CC2=O)CCO1] | 0.82 | 507.4 |
| 32 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 2-methyl-5-(trifluoromethyl)- pyrazole-3-carboxamide trifluoroacetic acid salt | 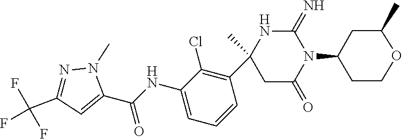 [SMILES: C[C@@H]1C[C@H](N2C(=N)N[C@](C)(c3cccc(NC(=O)c4cc(C(F)(F)F)nn4C)c3Cl)CC2=O)CCO1] | 0.81 | 527.4 |
| 33 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- quinoline-3-carboxamide trifluoroacetic acid salt | 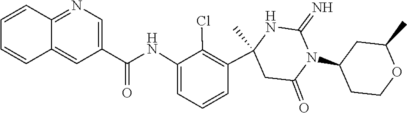 [SMILES: C[C@@H]1C[C@H](N2C(=N)N[C@](C)(c3cccc(NC(=O)c4cnc5ccccc5c4)c3Cl)CC2=O)CCO1] | 0.71 | 506.4 |
| 34 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 5-methylpyrazine-2-carboxamide trifluoroacetic acid salt | 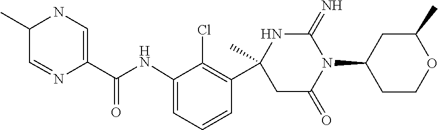 | 0.69 | 471.4 |
| 35 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 2-fluorobenzamide trifluoroacetic acid salt | 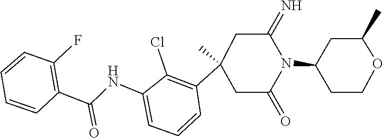 [SMILES: C[C@@H]1C[C@H](N2C(=N)C[C@](C)(c3cccc(NC(=O)c4ccccc4F)c3Cl)CC2=O)CCO1] | 0.76 | 473.4 |
| 36 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 3,4-difluorobenzamide trifluoroacetic acid salt | 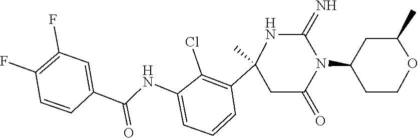 [SMILES: C[C@@H]1C[C@H](N2C(=N)N[C@](C)(c3cccc(NC(=O)c4ccc(F)c(F)c4)c3Cl)CC2=O)CCO1] | 0.77 | 491.4 |
| 37 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 4-(trifluoromethyl)benzamide trifluoroacetic acid salt | 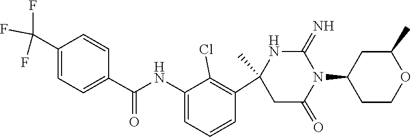 [SMILES: C[C@@H]1C[C@H](N2C(=N)N[C@](C)(c3cccc(NC(=O)c4ccc(C(F)(F)F)cc4)c3Cl)CC2=O)CCO1] | 0.86 | 523.4 |
| 38 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- pyrimidine-5-carboxamide trifluoroacetic acid salt | 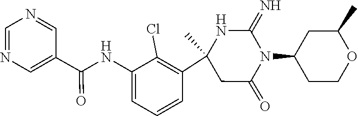 [SMILES: C[C@@H]1C[C@H](N2C(=N)N[C@](C)(c3cccc(NC(=O)c4cncnc4)c3Cl)CC2=O)CCO1] | 0.47 | 457.3 |
| 39 | 5-Chloro-N-(2-chloro-3-{(4S)-2- imino-4-methyl-1-[(2R*,4R*)-2- methyltetrahydropyran-4-yl]-6-oxo- hexahydropyrimidin-4-yl}phenyl)- pyridine-3-carboxamide trifluoroacetic acid salt | 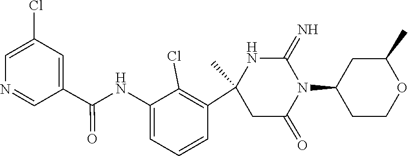 [SMILES: C[C@@H]1C[C@H](N2C(=N)N[C@](C)(c3cccc(NC(=O)c4cncc(Cl)c4)c3Cl)CC2=O)CCO1] | 0.67 | 490.3 |
| 40 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 5-fluoropyridine-3-carboxamide trifluoroacetic acid salt | 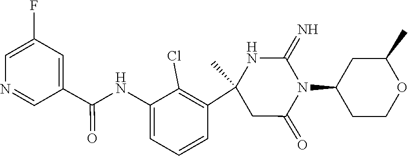 [SMILES: C[C@@H]1C[C@H](N2C(=N)N[C@](C)(c3cccc(NC(=O)c4cncc(F)c4)c3Cl)CC2=O)CCO1] | 0.60 | 474.4 |
| 41 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 3-(trifluoromethyl)benzamide trifluoroacetic acid salt | 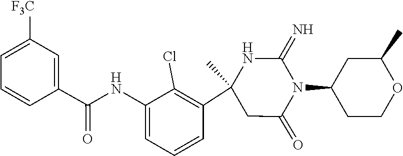 [SMILES: C[C@@H]1C[C@H](N2C(=N)N[C@](C)(c3cccc(NC(=O)c4cccc(C(F)(F)F)c4)c3Cl)CC2=O)CCO1] | 0.86 | 523.4 |
| 42 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 3,5-difluorobenzamide trifluoroacetic acid salt | 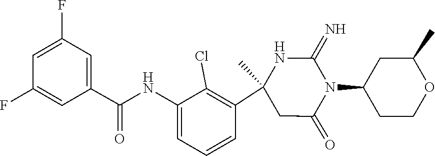 [SMILES: C[C@@H]1C[C@H](N2C(=N)N[C@](C)(c3cccc(NC(=O)c4cc(F)cc(F)c4)c3Cl)CC2=O)CCO1] | 0.77 | 491.3 |
| 43 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 1,6-naphthyridine-3-carboxamide trifluoroacetic acid salt | 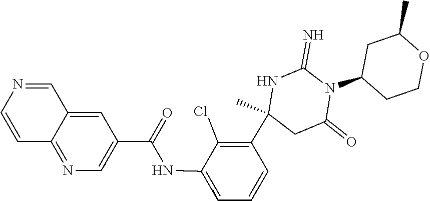 | 0.56 | 507.4 |
| 44 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 7-fluorobenzofuran-2-carboxamide trifluoroacetic acid salt | 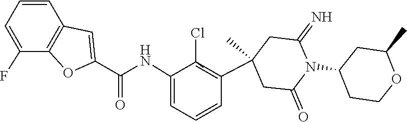 | 0.86 | 513.4 |
| 45 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 4-methoxypyridine-3-carboxamide trifluoroacetic acid salt | 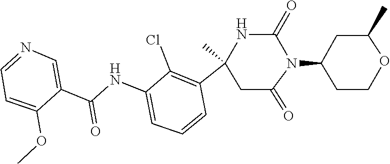 | 0.53 | 486.4 |
| 46 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- cinnoline-3-carboxamide trifluoroacetic acid salt | 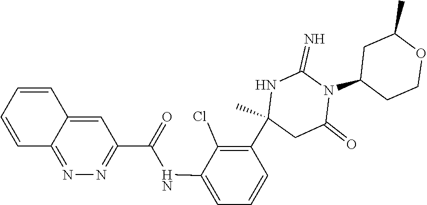 [SMILES: C[C@@H]1C[C@H](N2C(=N)N[C@](C)(c3cccc(NC(=O)c4cc5ccccc5nn4)c3Cl)CC2=O)CCO1] | 0.79 | 507.4 |
| 47 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 5-(trifluoromethoxy)pyridine-2- carboxamide trifluoroacetic acid salt | 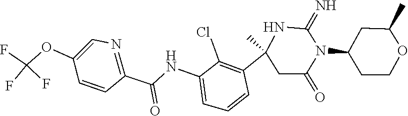 [SMILES: C[C@@H]1C[C@H](N2C(=N)N[C@](C)(c3cccc(NC(=O)c4ccc(OC(F)(F)F)cn4)c3Cl)CC2=O)CCO1] | 0.93 | 540.4 |
| 48 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- imidazo[1,2-alpyrazine-8- carboxamide trifluoroacetic acid salt | 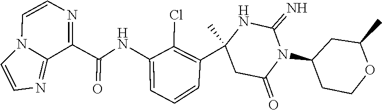 [SMILES: C[C@@H]1C[C@H](N2C(=N)N[C@](C)(c3cccc(NC(=O)c4nccn5ccnc45)c3Cl)CC2=O)CCO1] | 0.57 | 496.4 |
| 49 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 3-(trifluoromethyl)pyridine-2- carboxamide trifluoroacetic acid salt | 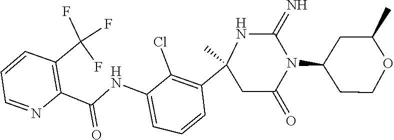 [SMILES: C[C@@H]1C[C@H](N2C(=N)N[C@](C)(c3cccc(NC(=O)c4ncccc4C(F)(F)F)c3Cl)CC2=O)CCO1] | 0.78 | 524.4 |
| 50 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 3-methoxypyridine-2-carboxamide trifluoroacetic acid salt | 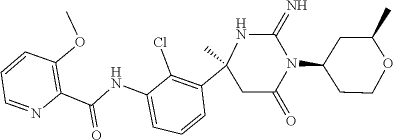 [SMILES: COc1cccnc1C(=O)Nc1cccc([C@]2(C)CC(=O)N([C@@H]3CCO[C@H](C)C3)C(=N)N2)c1Cl] | 0.64 | 486.4 |
| 51 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 2-methylpyrazole-3-carboxamide trifluoroacetic acid salt | 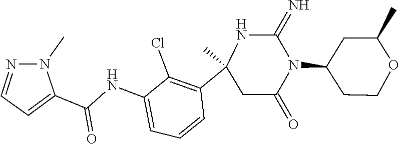 [SMILES: C[C@@H]1C[C@H](N2C(=N)N[C@](C)(c3cccc(NC(=O)c4ccnn4C)c3Cl)CC2=O)CCO1] | 0.59 | 459.4 |
| 52 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 1,5-naphthyridine-3-carboxamide trifluoroacetic acid salt | 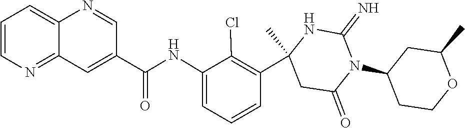 [SMILES: C[C@@H]1C[C@H](N2C(=N)N[C@](C)(c3cccc(NC(=O)c4cnc5cccnc5c4)c3Cl)CC2=O)CCO1] | 0.60 | 507.4 |
| 53 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 6-cyclopropylpyridine-3- carboxamide hydrochloride | 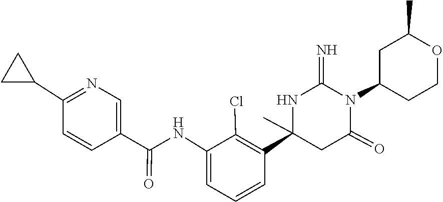 [SMILES: C[C@@H]1C[C@H](N2C(=N)N[C@](C)(c3cccc(NC(=O)c4ccc(C5CC5)nc4)c3Cl)CC2=O)CCO1] | 0.70 | 496.4 |
| 54 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 6-methylpyridazine-3-carboxamide hydrochloride | 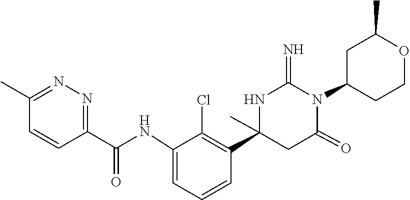 [SMILES: Cc1ccc(C(=O)Nc2cccc([C@]3(C)CC(=O)N([C@@H]4CCO[C@H](C)C4)C(=N)N3)c2Cl)nn1] | 0.67 | 471.4 |
| 55 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 5-(trifluoromethyl)pyridazine-3- carboxamide hydrochloride | 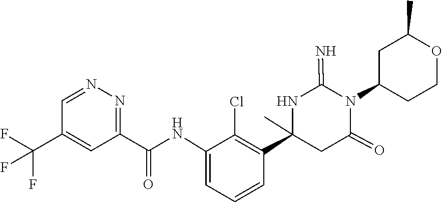 [SMILES: C[C@@H]1C[C@H](N2C(=N)N[C@](C)(c3cccc(NC(=O)c4cc(C(F)(F)F)cnn4)c3Cl)CC2=O)CCO1] | 0.80 | 525.4 |
| 56 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 4-(trifluoromethyl)pyridine-3- carboxamide hydrochloride | 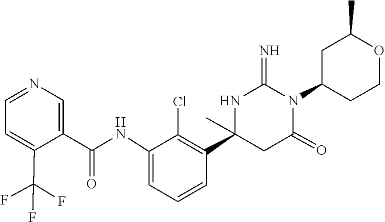 [SMILES: C[C@@H]1C[C@H](N2C(=N)N[C@](C)(c3cccc(NC(=O)c4cnccc4C(F)(F)F)c3Cl)CC2=O)CCO1] | 0.67 | 524.4 |
| 57 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 2-cyclopropylpyridine-3- carboxamide hydrochloride | 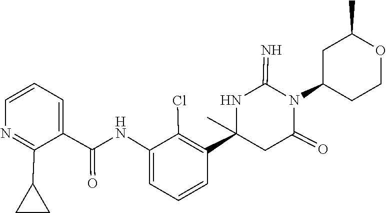 [SMILES: C[C@@H]1C[C@H](N2C(=N)N[C@](C)(c3cccc(NC(=O)c4cccnc4C4CC4)c3Cl)CC2=O)CCO1] | 0.65 | 496.4 |
| 58 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 6-(2,2,2-trifluoroethoxy)pyridine-3- carboxamide hydrochloride | 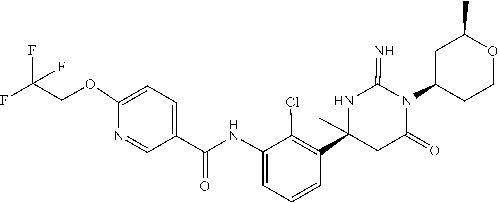 [SMILES: C[C@@H]1C[C@H](N2C(=N)N[C@](C)(c3cccc(NC(=O)c4ccc(OCC(F)(F)F)nc4)c3Cl)CC2=O)CCO1] | 0.85 | 554.4 |
| 59 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 6-(trifluoromethyl)pyridazine-3- carboxamide hydrochloride | 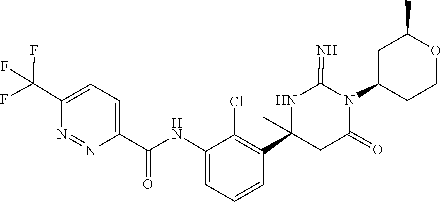 [SMILES: C[C@@H]1C[C@H](N2C(=N)N[C@](C)(c3cccc(NC(=O)c4ccc(C(F)(F)F)nn4)c3Cl)CC2=O)CCO1] | 0.79 | 525.4 |
| 60 | N-(2-Chloro-3-{(4S)-2-imino-4- methyl-1-[(2,R*,4R*)-2-methyl- tetrahydropyran-4-yl]-6-oxo- hexahydropyrimidin-4-yl}phenyl)- imidazo[1,5-a]pyridine-1- carboxamide hydrochloride | 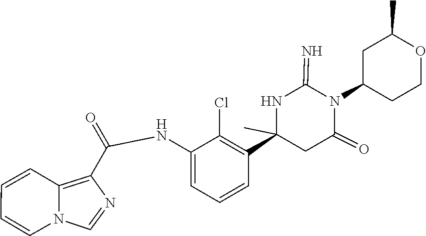 [SMILES: C[C@@H]1C[C@H](N2C(=N)N[C@](C)(c3cccc(NC(=O)c4ncn5ccccc45)c3Cl)CC2=O)CCO1] | 0.74 | 495.4 |
| 61 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 2-(trifluoromethyl)pyridine-3- carboxamide hydrochloride | 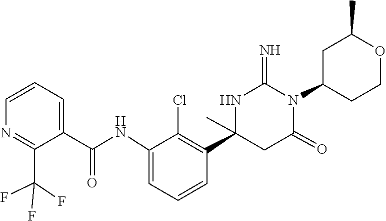 [SMILES: C[C@@H]1C[C@H](N2C(=N)N[C@](C)(c3cccc(NC(=O)c4cccnc4C(F)(F)F)c3Cl)CC2=O)CCO1] | 0.66 | 524.4 |
| 62 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- [1,2,4]triazolo[4,3-a]pyridine-3- carboxamide hydrochloride | 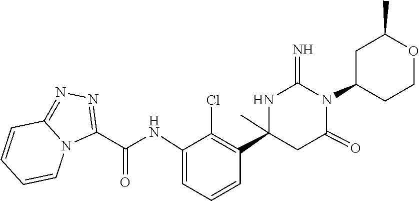 [SMILES: C[C@@H]1C[C@H](N2C(=N)N[C@](C)(c3cccc(NC(=O)c4nnc5ccccn45)c3Cl)CC2=O)CCO1] | 0.67 | 496.4 |
| 63 | 5-Chloro-N-(2-chloro-3-{(4S)-2- imino-4-methyl-1-[(2R*,4R*)-2- methyltetrahydropyran-4-yl]-6-oxo- hexahydropyrimidin-4-yl]phenyl)- pyridine-2-carboxamide hydrochloride | 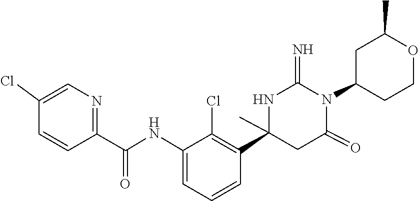 [SMILES: C[C@@H]1C[C@H](N2C(=N)N[C@](C)(c3cccc(NC(=O)c4ccc(Cl)cn4)c3Cl)CC2=O)CCO1] | 0.85 | 490.4 |
| 64 | N-(2-Chloro-3-{(4S)-2-imino-4- methyl-1-[(2,R*,4R*)-2-methyl- tetrahydropyran-4-yl]-6-oxo- hexahydropyrimidin-4-yl}phenyl)- 2,4,5-trifluorobenzamide hydrochloride | 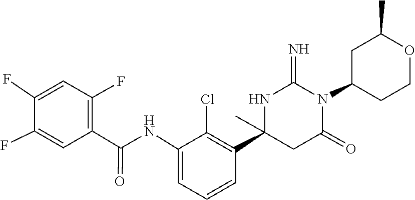 [SMILES: C[C@@H]1C[C@H](N2C(=N)N[C@](C)(c3cccc(NC(=O)c4cc(F)c(F)cc4F)c3Cl)CC2=O)CCO1] | 0.82 | 509.4 |
| 65 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- imidazo[1,5-a]pyridine-3- carboxamide hydrochloride | 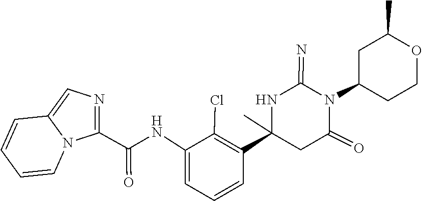 | 0.84 | 495.1 |
| 66 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 6-methylpyrazine-2-carboxamide hydrochloride | 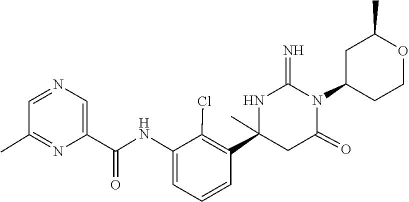 [SMILES: Cc1cncc(C(=O)Nc2cccc([C@]3(C)CC(=O)N([C@@H]4CCO[C@H](C)C4)C(=N)N3)c2Cl)n1] | 0.70 | 471.4 |
| 67 | 4-Chloro-N-(2-chloro-3-{(4S)-2- imino-4-methyl-1-[(2R*,4R*)-2- methyltetrahydropyran-4-yl]-6-oxo- hexahydropyrimidin-4-yl}phenyl)- 2-fluorobenzamide hydrochloride | 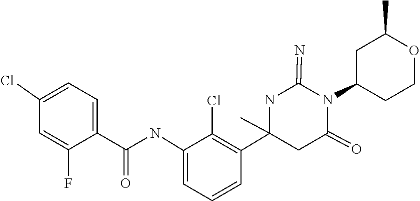 | 0.84 | 507.1 |
| 68 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 6-methoxypyrazine-2-carboxamide hydrochloride | 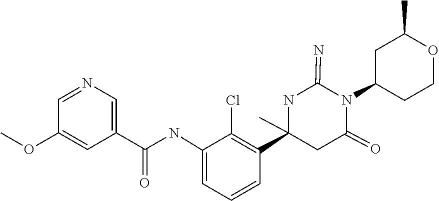 | 0.71 | 487.1 |
| 69 | 5-Chloro-N-(2-chloro-3-{(4S)-2- imino-4-methyl-1-[(2R*,4R*)-2- methyltetrahydropyran-4-yl]-6-oxo- hexahydropyrimidin-4-yl}phenyl)- 2,4-difluorobenzamide hydrochloride | 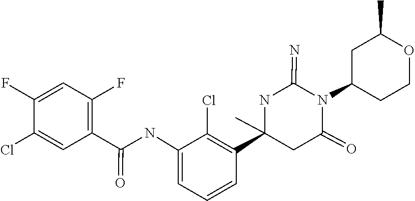 | 0.85 | 525.1 |
| 70 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 5-(trifluoromethyl)pyridine-3- carboxamide hydrochloride | 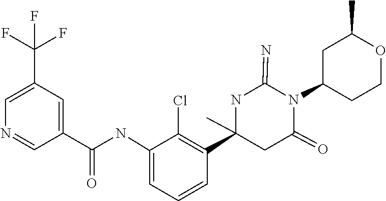 | 0.71 | 524.1 |
| 71 | 3-Chloro-N-(2-chloro-3-{(4S)-2- imino-4-methyl-1-[(2R*,4R*)-2- methyltetrahydropyran-4-yl]-6-oxo- hexahydropyrimidin-4-yl}phenyl)- 2,4-difluorobenzamide hydrochloride | 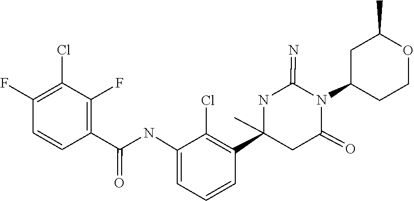 | 0.83 | 525.1 |
| 72 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 6-cyanopyridine-2-carboxamide hydrochloride | 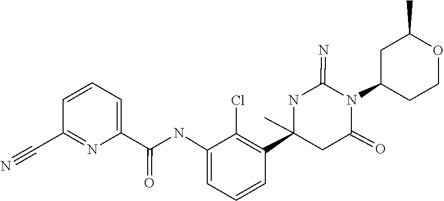 | 0.72 | 481.1 |
| 73 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 3-cyano-2-fluorobenzamide hydrochloride | 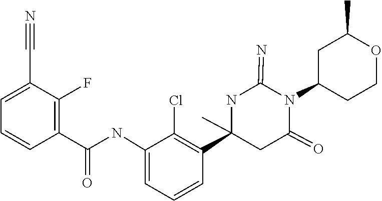 | 0.70 | 498.1 |
| 74 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 5-cyanopyridine-3-carboxamide hydrochloride | 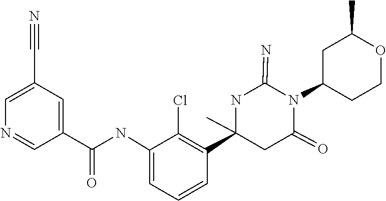 | 0.56 | 481.1 |
| 75 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 4-cyanopyridine-2-carboxamide hydrochloride | 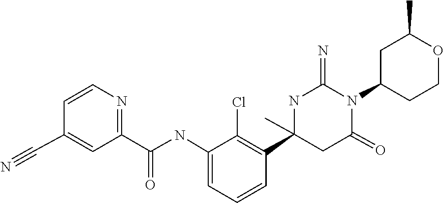 | 0.73 | 481.4 |
| 76 | N-(2-Chloro-3-{(4S)-2-imino-4- methyl-1-{(2R*,4R*)-2-methyl- tetrahydropyran-4-yl]-6-oxo- hexahydropyrimidin-4-yl}phenyl)- 5-cyano-2-fluorobenzamide hydrochloride | 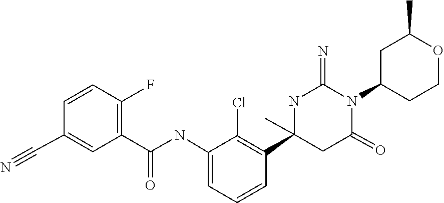 | 0.70 | 498.3 |
| 77 | 3-Chloro-N-(2-chloro-3-{(4S)-2- imino-4-methyl-1-[(2R*,4R*)-2- methyltetrahydropyran-4-yl]-6-oxo- hexahydropyrimidin-4-yl}phenyl)- 5-cyanobenzamide hydrochloride | 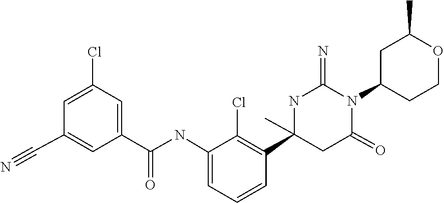 | 0.78 | 514.3 |
| 78 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 4-methylpyrimidine-2-carboxamide hydrochloride | 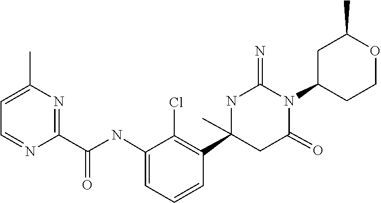 | 0.60 | 471.1 |
| 79 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 2-methoxynaphthalene-1- carboxamide trifluoroacetic acid salt | 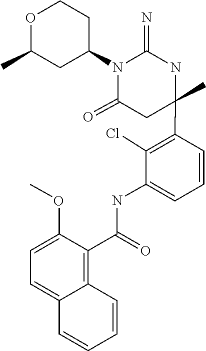 | 0.83 | 535.4 |
| 80 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 4-fluoro-2-(trifluoromethyl)- benzamide trifluoroacetic acid salt | 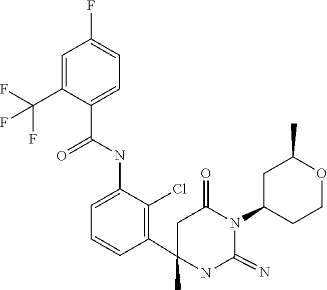 | 0.82 | 541.4 |
| 81 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 2-(methylsulfonyl)benzamide trifluoroacetic acid salt | 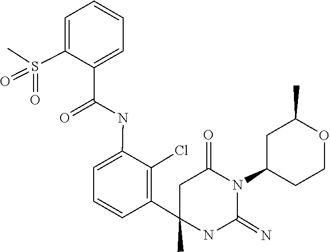 | 0.62 | 533.4 |
| 82 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 4-methylpyridine-3-carboxamide trifluoroacetic acid salt | 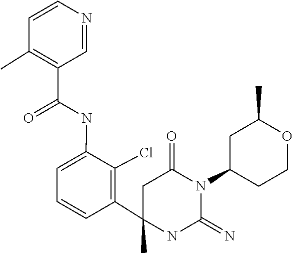 | 0.52 | 470.3 |
| 83 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 2-fluoro-5-(trifluoromethyl)- benzamide trifluoroacetic acid salt | 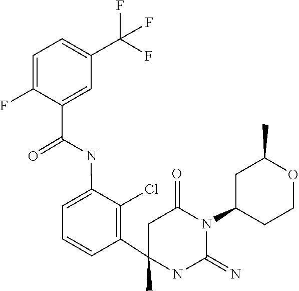 | 0.90 | 541.4 |
| 84 | 5-Chloro-N-(2-chloro-3-{(4S)-2- imino-4-methyl-1-[(2R*,4R*)-2- methyltetrahydropyran-4-yl]-6-oxo- hexahydropyrimidin-4-yl]phenyl)- 2-(difluoromethoxy)benzamide trifluoroacetic acid salt | 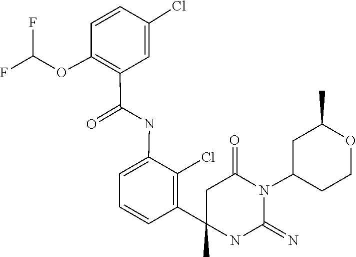 | 0.89 | 555.4 |
| 85 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 2-methoxy-5-(methylsulfonyl)- benzamide trifluoroacetic acid salt | 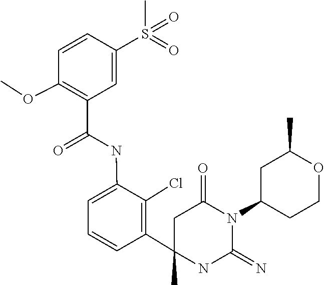 | 0.69 | 563.4 |
| 86 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 1,3-benzodioxole-4-carboxamide trifluoroacetic acid salt | 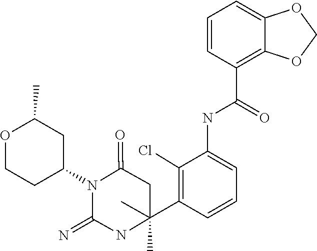 | 0.8 | 499.4 |
| 87 | N-(2-Chloro-3-{(4S)-2-imino-4- methyl-1-[(2,R*,4R*)-2-methyl- tetrahydropyran-4-yl]-6-oxo- hexahydropyrimidin-4-yl}phenyl)- 5-fluoro-2-methoxybenzamide trifluoroacetic acid salt | 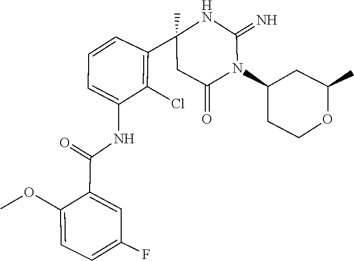 | 0.87 | 503.4 |
| 88 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 3-(trifluoromethyl)quinoxaline-2- carboxamide trifluoroacetic acid salt | 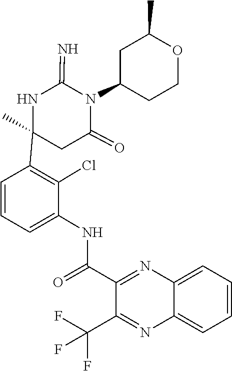 [SMILES: C[C@@H]1C[C@H](N2C(=N)N[C@](C)(c3cccc(NC(=O)c4nc5ccccc5nc4C(F)(F)F)c3Cl)CC2=O)CCO1] | 0.94 | 575.4 |
| 89 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 3-(trifluoromethyl)pyridine-4- carboxamide trifluoroacetic acid salt | 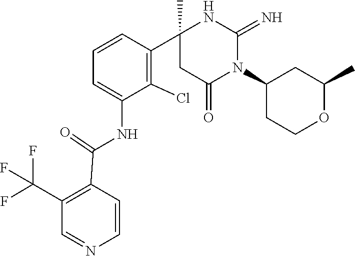 [SMILES: C[C@@H]1C[C@H](N2C(=N)N[C@](C)(c3cccc(NC(=O)c4ccncc4C(F)(F)F)c3Cl)CC2=O)CCO1] | 0.65 | 524.2 |
| 90 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 3-(pyrazol-1-yl)benzamide trifluoroacetic acid salt | 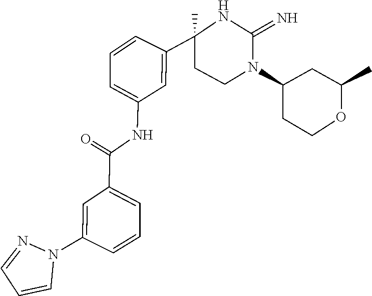 [SMILES: C[C@@H]1C[C@H](N2CC[C@@](C)(c3cccc(NC(=O)c4cccc(-n5cccn5)c4)c3)NC2=N)CCO1] | 0.76 | 521.2 |
| 91 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 6-(dimethylamino)pyrazine-2- carboxamide hydrochloride | 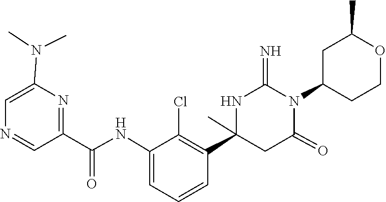 [SMILES: C[C@@H]1C[C@H](N2C(=N)N[C@](C)(c3cccc(NC(=O)c4cncc(N(C)C)n4)c3Cl)CC2=O)CCO1] | 0.75 | 500.2 |
| 92 | 5-Chloro-N-(2-chloro-3-{(4S)-2- imino-4-methyl-1-[(2R*,4R*)-2- methyltetrahydropyran-4-yl]-6-oxo- hexahydropyrimidin-4-yl]phenyl)- pyrimidine-2-carboxamide hydrochloride | 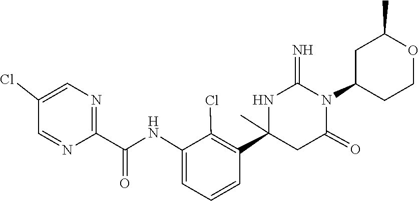 [SMILES: C[C@@H]1C[C@H](N2C(=N)N[C@](C)(c3cccc(NC(=O)c4ncc(Cl)cn4)c3Cl)CC2=O)CCO1] | 0.65 | 491.1 |
| 93 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 4-(morpholin-4-yl)pyridine-2- carboxamide hydrochloride | 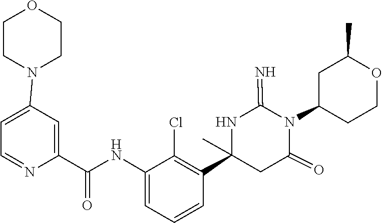 [SMILES: C[C@@H]1C[C@H](N2C(=N)N[C@](C)(c3cccc(NC(=O)c4cc(N5CCOCC5)ccn4)c3Cl)CC2=O)CCO1] | 0.67 | 541.3 |
| 94 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 5-fluoro-3-(trifluoromethyl)- pyridine-2-carboxamide hydrochloride | 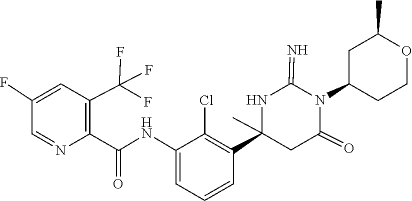 [SMILES: C[C@@H]1C[C@H](N2C(=N)N[C@](C)(c3cccc(NC(=O)c4ncc(F)cc4C(F)(F)F)c3Cl)CC2=O)CCO1] | 0.82 | 542.1 |
| 95 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 3-cyano-5-(trifluoromethyl)- benzamide hydrochloride | 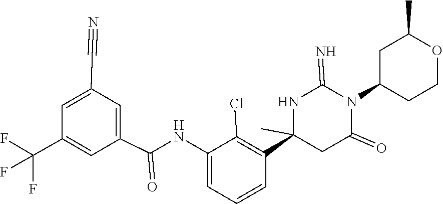 [SMILES: C[C@@H]1C[C@H](N2C(=N)N[C@](C)(c3cccc(NC(=O)c4cc(C#N)cc(C(F)(F)F)c4)c3Cl)CC2=O)CCO1] | 0.84 | 548.4 |
| 96 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 4-fluoropyridine-3-carboxamide hydrochloride | 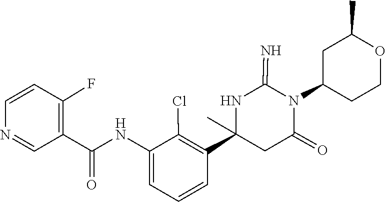 [SMILES: C[C@@H]1C[C@H](N2C(=N)N[C@](C)(c3cccc(NC(=O)c4cnccc4F)c3Cl)CC2=O)CCO1] | 0.59 | 474.1 |
| 97 | 3-Chloro-N-(2-chloro-3-{(4S)-2- imino-4-methyl-1-[(2R* ,4R*)-2- methyltetrahydropyran-4-yl]-6-oxo- hexahydropyrimidin-4-yl]phenyl)- 2-fluorobenzamide hydrochloride | 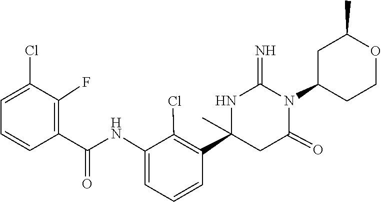 [SMILES: C[C@@H]1C[C@H](N2C(=N)N[C@](C)(c3cccc(NC(=O)c4cccc(Cl)c4F)c3Cl)CC2=O)CCO1] | 0.81 | 507.1 |
| 98 | 2-Chloro-N-(2-chloro-3-{(4S)-2- imino-4-methyl-1-[(2R*,4R*)-2- methyltetrahydropyran-4-yl]-6-oxo- hexahydropyrimidin-4-yl}phenyl)- 5-cyanobenzamide hydrochloride | 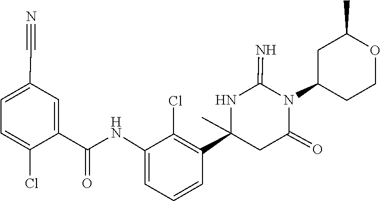 [SMILES: C[C@@H]1C[C@H](N2C(=N)N[C@](C)(c3cccc(NC(=O)c4cc(C#N)ccc4Cl)c3Cl)CC2=O)CCO1] | 0.69 | 514.1 |
| 99 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 3-cyano-5-fluorobenzamide hydrochloride | 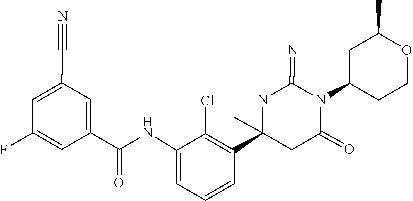 [SMILES: C[C@@H]1C[C@H](N2C(=N)N[C@](C)(c3cccc(NC(=O)c4cc(F)cc(C#N)c4)c3Cl)CC2=O)CCO1] | 0.72 | 498.2 |
| 100 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 3,5-difluoropyridine-2-carboxamide hydrochloride | 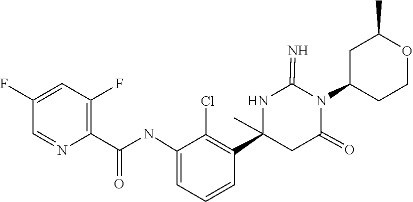 [SMILES: C[C@@H]1C[C@H](N2C(=N)N[C@](C)(c3cccc(NC(=O)c4ncc(F)cc4F)c3Cl)CC2=O)CCO1] | 0.72 | 492.2 |
| 101 | N-(2-Chloro-3-{(4S)-2-imino-4- methyl-1-[(2,R*,4R*)-2-methyl- tetrahydropyran-4-yl]-6-oxo- hexahydropyrimidin-4-yl]phenyl)- 2,4,6-trifluorobenzamide hydrochloride | 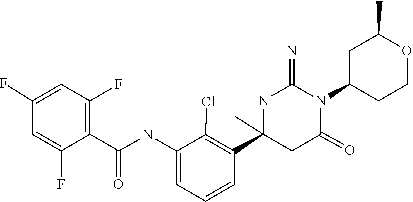 | 0.72 | 509.2 |
| 102 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 4-fluoro-2-(trifluoromethoxy)- benzamide hydrochloride | 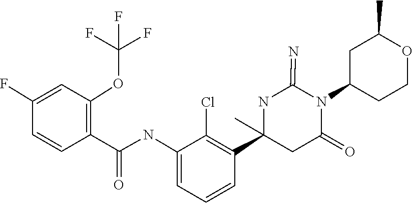 | 0.85 | 557.2 |
| 103 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 3-methylimidazole-4-carboxamide hydrochloride | 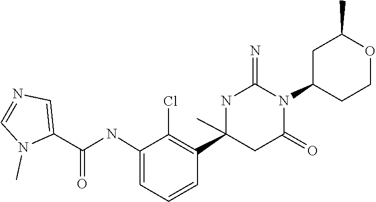 | 0.41 | 459.3 |
| 104 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 5-cyclopropylpyridine-3- carboxamide hydrochloride | 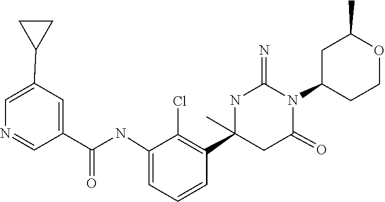 | 0.68 | 496.2 |
| 105 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 3-(imidazol-1-yl)benzamide hydrochloride | 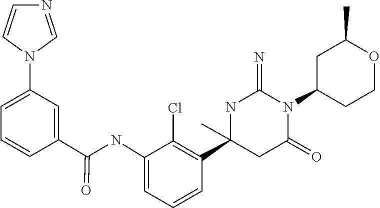 | 0.43 | 521.3 |
| 106 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 5-cyano-2-methoxybenzamide hydrochloride | 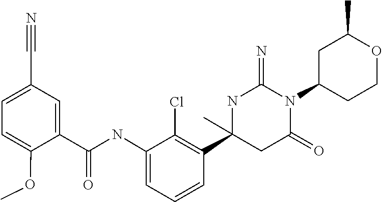 | 0.78 | 510.4 |
| 107 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 3-cyano-2-methoxybenzamide hydrochloride | 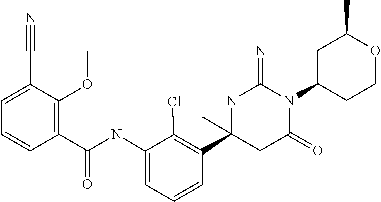 | 0.77 | 510.5 |
| 108 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 5-(prop-1-ynyl)pyridine-3- carboxamide hydrochloride | 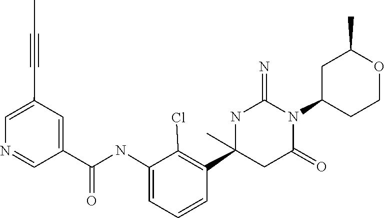 | 0.70 | 494.2 |
| 109 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 3-(prop-1-ynyl)benzamide hydrochloride | 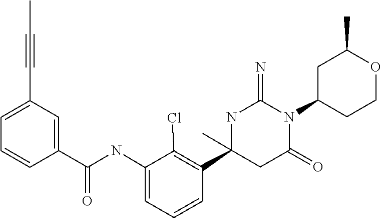 | 0.85 | 493.4 |
| 110 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 3-(4-methylimidazol-1-yl)- benzamide hydrochloride | 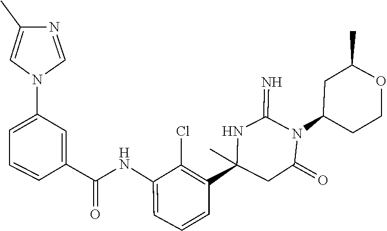 [SMILES: Cc1cn(-c2cccc(C(=O)Nc3cccc([C@]4(C)CC(=O)N([C@@H]5CCO[C@H](C)C5)C(=N)N4)c3Cl)c2)cn1] | 0.44 | 533.5 |
| 111 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 2-methyl-4-(trifluoromethyl)- pyridine-3-carboxamide hydrochloride | 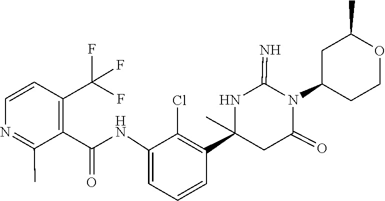 [SMILES: Cc1nccc(C(F)(F)F)c1C(=O)Nc1cccc([C@]2(C)CC(=O)N([C@@H]3CCO[C@H](C)C3)C(=N)N2)c1Cl] | 0.75 | 537.9 |
| 112 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 1-oxidopyridin-1-ium-3- carboxamide hydrochloride | 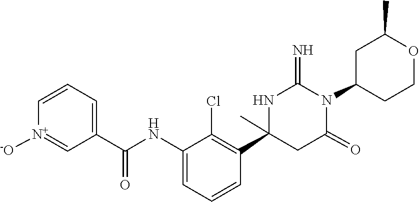 | 0.45 | 472.4 |
| 113 | N-(2-Chloro-3-{(4S)-2-imino-4- methyl-1-[(2R*,4R*)-2-methyl- tetrahydropyran-4-yl]-6-oxo- hexahydropyrimidin-4-yl}phenyl)- 4-cyano-1-methylpyrrole-2- carboxamide trifluoroacetic acid salt | 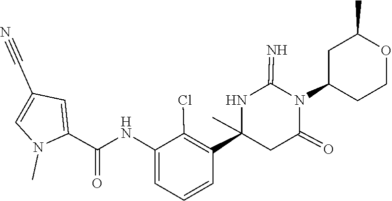 [SMILES: C[C@@H]1C[C@H](N2C(=N)N[C@](C)(c3cccc(NC(=O)c4cc(C#N)cn4C)c3Cl)CC2=O)CCO1] | 0.66 | 483.4 |
| 114 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 2-methoxy-4-(trifluoromethyl)- pyridine-3-carboxamide hydrochloride | 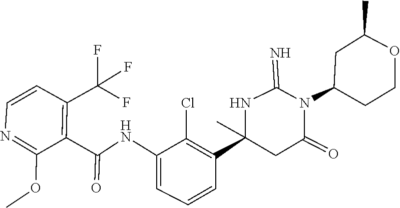 [SMILES: COc1nccc(C(F)(F)F)c1C(=O)Nc1cccc([C@]2(C)CC(=O)N([C@@H]3CCO[C@H](C)C3)C(=N)N2)c1Cl] | 0.75 | 554.5 |
| 115 | N-(2-Chloro-3-{(4S)-2-imino-4- methyl-1-[(2R*,4R*)-2-methyl- tetrahydropyran-4-yl]-6-oxo- hexahydropyrimidin-4-yl]phenyl)- 5-cyanofuran-2-carboxamide trifluoroacetic acid salt | 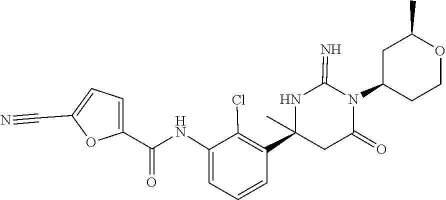 [SMILES: C[C@@H]1C[C@H](N2C(=N)N[C@](C)(c3cccc(NC(=O)c4ccc(C#N)o4)c3Cl)CC2=O)CCO1] | 0.65 | 470.1 |
| 116 | N-(2-Chloro-3-{(4S)-2-imino-4- methyl-1-[(2R* ,4R* )-2-methyl- tetrahydropyran-4-yl]-6-oxo- hexahydropyrimidin-4-yl]phenyl)- 3-cyano-4-fluorobenzamide trifluoroacetic acid salt | 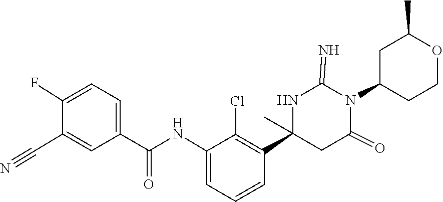 [SMILES: C[C@@H]1C[C@H](N2C(=N)N[C@](C)(c3cccc(NC(=O)c4ccc(F)c(C#N)c4)c3Cl)CC2=O)CCO1] | 0.72 | 498.5 |
| 117 | 4-Chloro-N-(2-chloro-3-{(4S)-2- imino-4-methyl-1-[(2R*,4R*)-2- methyltetrahydropyran-4-yl]-6-oxo- hexahydropyrimidin-4-yl}phenyl)- 3-cyanobenzamide trifluoroacetic acid salt | 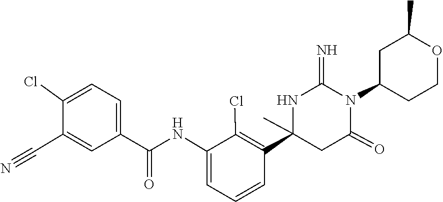 [SMILES: C[C@@H]1C[C@H](N2C(=N)N[C@](C)(c3cccc(NC(=O)c4ccc(Cl)c(C#N)c4)c3Cl)CC2=O)CCO1] | 0.77 | 514.4 |

1. A compound of formula (I) or an N-oxide thereof, or a pharmaceutically acceptable salt thereof: 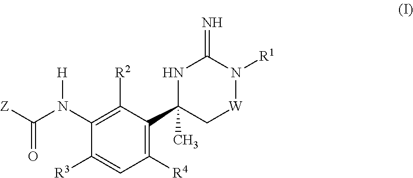
wherein
W represents C(O) or S(O)₂;
Z represents aryl or heteroaryl, either of which groups may be optionally substituted by one or more substituents;
R¹ represents C₃₋₇ cycloalkyl, aryl(C₁₋₆)alkyl, C₃₋₇ heterocycloalkyl, C₃₋₇ heterocycloalkyl(C₁₋₆)alkyl, C₄₋₉ heterobicycloalkyl, C₄₋₉ spiroheterocycloalkyl or heteroaryl(C₁₋₆)alkyl, any of which groups may be optionally substituted by one or more substituents; and
R², R³ and R⁴ independently represent hydrogen, halogen or trifluoromethyl.

2. A compound as claimed in claim 1 represented by formula (IIA), or a pharmaceutically acceptable salt thereof: 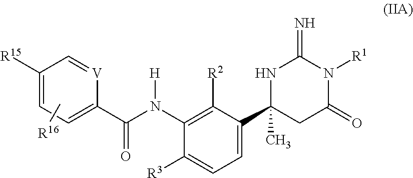
wherein
V represents N or CH;
R¹⁵ and R¹⁶ independently represent hydrogen, halogen, cyano, nitro, C₁₋₆ alkyl, difluoromethyl, trifluoromethyl, hydroxy, hydroxy(C₁₋₆)alkyl, C₁₋₆ alkoxy, difluoro-methoxy, trifluoromethoxy, phenoxy, C₁₋₆ alkylthio, C₁₋₆ alkylsulfinyl, C₁₋₆ alkylsulfonyl, amino, C₁₋₆ alkylamino, di(C₁₋₆)alkylamino, amino(C₁₋₆)alkyl, di(C₁₋₆)alkylamino(C₁₋₆)-alkyl, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkyl-aminocarbonyl, di(C₁₋₆)alkylaminocarbonyl, aminosulfonyl, C₁₋₆ alkylaminosulfonyl, di(C₁-6)alkylaminosulfonyl or di(C₁₋₆)alkylsulfoximino; and
R¹, R² and R³ are as defined in claim 1.

3. A compound as claimed in claim 2 wherein R¹⁵ represents hydrogen, halogen, cyano, trifluoromethyl or trifluoromethoxy.

4. A compound as claimed in claim 2 wherein R¹⁶ represents hydrogen, halogen, cyano, trifluoromethyl, C₁₋₆ alkoxy, trifluoromethoxy or C₁₋₆ alkylsulfonyl.

5. A compound as claimed in claim 1 wherein R¹ represents C₃₋₇ cycloalkyl or C₃₋₇ heterocycloalkyl, either of which groups may be optionally substituted by one, two or three substituents independently selected from halogen and C₁₋₆ alkyl.

6. A compound as claimed in claim 1 represented by formula (IIB), or a pharmaceutically acceptable salt thereof: 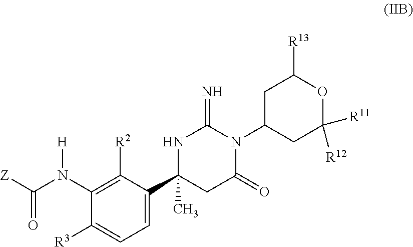
wherein
R¹¹ represents hydrogen or methyl;
R¹² represents hydrogen or methyl;
R¹³ represents hydrogen or methyl; and
Z, R² and R³ are as defined in claim 1.

7. A compound as claimed in claim 1 represented by formula (IIC), or a pharmaceutically acceptable salt thereof: 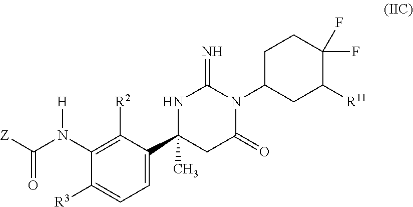
wherein
R¹¹ represents hydrogen or methyl.

8. A compound as claimed in claim 1, wherein Z represents phenyl, naphthyl, furyl, benzofuryl, pyrrolyl, indolyl, pyrazolyl, imidazolyl, imidazo[1,2-a]pyridinyl, imidazo[1,5-a]pyridinyl, imidazo[1,2-a]pyrazinyl, oxadiazolyl, [1,2,4]-triazolo[4,3-a]pyridinyl, tetrazolo[1,5-a]pyridinyl, pyridinyl, quinolinyl, naphthyridinyl, pyridazinyl, cinnolinyl, pyrimidinyl, pyrazinyl or quinoxalinyl, any of which groups may be optionally substituted by one, two or three substituents independently selected from halogen, cyano, C₁₋₆ alkyl, trifluoromethyl, C₂₋₆ alkynyl, cyclopropyl, C₁₋₆ alkoxy, difluoromethoxy, trifluoromethoxy, trifluoroethoxy, methylenedioxy, C₁₋₆ alkylsulfonyl, di(C₁₋₆)alkylamino, morpholinyl, pyrazolyl, imidazolyl and (C₁-6)alkylimidazolyl.

9. A compound as claimed in claim 1 wherein R² represents chloro.

10. A compound as claimed in claim 1 which is
N-{2-Chloro-3-[(4S)-2-imino-4-methyl-6-oxo-1-(tetrahydropyran-4-yl)hexahydropyrimidin-4-yl]-phenyl}benzamide trifluoroacetic acid salt;
5-Chloro-N-{2-chloro-3-[(4S)-2-imino-4-methyl-6-oxo-1-(tetrahydropyran-4-yl)hexahydro-pyrimidin-4-yl]phenyl}pyridine-2-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-4-fluorobenzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2,4-difluorobenzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-pyridine-4-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-pyridine-2-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-pyridine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-1-methylpyrazole-3-carboxamide hydrochloride;
4-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-benzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-4-cyanobenzamide hydrochloride;
3-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-benzamide hydrochloride;
2-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-benzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-fluoropyridine-2-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-pyrazine-2-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-pyrimidine-2-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-pyrimidine-4-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-4-methyl-1,2,5-oxadiazole-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-(trifluoromethyl)pyridine-2-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-pyridazine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-cyanobenzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-6-(trifluoromethyl)pyridine-3-carboxamide hydrochloride;
3-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-5-methylbenzamide hydrochloride;
N-{2-Chloro-3-[(4S)-1-(4,4-difluorocyclohexyl)-2-imino-4-methyl-6-oxohexahydropyrimidin-4-yl]phenyl}-3-cyanobenzamide hydrochloride;
N-(2-Chloro-3-{(4S)-1-[(2S*,6R*)-2,6-dimethyltetrahydropyran-4-yl]-2-imino-4-methyl-6-oxohexahydro-pyrimidin-4-yl}phenyl)-3-cyano-benzamide hydrochloride;
N-(2-Chloro-3-{(4S)-1-[(1R*,3R*)-4,4-difluoro-3-methylcyclohexyl]-2-imino-4-methyl-6-oxohexahydro-pyrimidin-4-yl}phenyl)-3-cyano-benzamide trifluoroacetic acid salt;
N-(2-Chloro-3-{(4S)-1-[(1 S*,3S*)-4,4-difluoro-3-methylcyclohexyl]-2-imino-4-methyl-6-oxohexahydro-pyrimidin-4-yl}phenyl)-3-cyano-benzamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-1-methylindole-3-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-6-methylimidazo[1,2-a]pyridine-8-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-tetrazolo[1,5-a]pyridine-8-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2-methoxypyridine-3-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-quinoxaline-2-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2-methyl-5-(trifluoromethyl)-pyrazole-3-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-quinoline-3-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-methylpyrazine-2-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2-fluorobenzamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3,4-difluorobenzamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-4-(trifluoromethyl)benzamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-pyrimidine-5-carboxamide trifluoroacetic acid salt;
5-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-pyridine-3-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-fluoropyridine-3-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-(trifluoromethyl)benzamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3,5-difluorobenzamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-1,6-naphthyridine-3-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-7-fluorobenzofuran-2-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-4-methoxypyridine-3-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-cinnoline-3-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-(trifluoromethoxy)pyridine-2-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-imidazo[1,2-a]pyrazine-8-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-(trifluoromethyl)pyridine-2-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-methoxypyridine-2-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2-methylpyrazole-3-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-1,5-naphthyridine-3-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-6-cyclopropylpyridine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-6-methylpyridazine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-(trifluoromethyl)pyridazine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-4-(trifluoromethyl)pyridine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2-cyclopropylpyridine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-6-(2,2,2-trifluoroethoxy)pyridine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-6-(trifluoromethyl)pyridazine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-imidazo[1,5-a]pyridine-1-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2-(trifluoromethyl)pyridine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-[1,2,4]triazolo[4,3-a]pyridine-3-carboxamide hydrochloride;
5-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-pyridine-2-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2,4,5-trifluorobenzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-imidazo[1,5-a]pyridine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-6-methylpyrazine-2-carboxamide hydrochloride;
4-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-2-fluorobenzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-6-methoxypyrazine-2-carboxamide hydrochloride;
5-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-2,4-difluorobenzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-(trifluoromethyl)pyridine-3-carboxamide hydrochloride;
3-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-2,4-difluorobenzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-6-cyanopyridine-2-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-cyano-2-fluorobenzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-cyanopyridine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-4-cyanopyridine-2-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-cyano-2-fluorobenzamide hydrochloride;
3-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-5-cyanobenzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-4-methylpyrimidine-2-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2-methoxynaphthalene-1-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-4-fluoro-2-(trifluoromethyl)-benzamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2-(methylsulfonyl)benzamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-4-methylpyridine-3-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2-fluoro-5-(trifluoromethyl)-benzamide trifluoroacetic acid salt;
5-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-2-(difluoromethoxy)benzamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2-methoxy-5-(methylsulfonyl)-benzamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-1,3-benzodioxole-4-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-fluoro-2-methoxybenzamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-(trifluoromethyl)quinoxaline-2-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-(trifluoromethyl)pyridine-4-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-(pyrazol-1-yl)benzamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-6-(dimethylamino)pyrazine-2-carboxamide hydrochloride;
5-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-pyrimidine-2-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-4-(morpholin-4-yl)pyridine-2-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-fluoro-3-(trifluoromethyl)-pyridine-2-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-cyano-5-(trifluoromethyl)-benzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-4-fluoropyridine-3-carboxamide hydrochloride;
3-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-2-fluorobenzamide hydrochloride;
2-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-5-cyanobenzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-cyano-5-fluorobenzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3,5-difluoropyridine-2-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2,4,6-trifluorobenzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-4-fluoro-2-(trifluoromethoxy)-benzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-methylimidazole-4-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-cyclopropylpyridine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-(imidazol-1-yl)benzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-cyano-2-methoxybenzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-cyano-2-methoxybenzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-(prop-1-ynyl)pyridine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-(prop-1-ynyl)benzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-(4-methylimidazol-1-yl)-benzamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2-methyl-4-(trifluoromethyl)-pyridine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-1-oxidopyridin-1-ium-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-4-cyano-1-methylpyrrole-2-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-2-methoxy-4-(trifluoromethyl)-pyridine-3-carboxamide hydrochloride;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-5-cyanofuran-2-carboxamide trifluoroacetic acid salt;
N-(2-Chloro-3-{(4S)-2-imino-4-methyl-1-[(2R*,4R*)-2-methyl-tetrahydropyran-4-yl]-6-oxo-hexahydropyrimidin-4-yl}phenyl)-3-cyano-4-fluorobenzamide trifluoroacetic acid salt; or
4-Chloro-N-(2-chloro-3-{(4S)-2-imino-4-methyl-1-[(2R*,4R*)-2-methyltetrahydropyran-4-yl]-6-oxo-hexahydropyrimidin-4-yl}phenyl)-3-cyanobenzamide trifluoroacetic acid salt.

11-12. (canceled)

13. A pharmaceutical composition comprising a compound of formula (I) as defined in claim 1 or an N-oxide thereof, or a pharmaceutically acceptable salt thereof, in association with a pharmaceutically acceptable carrier.

14. (canceled)

15. A method for the treatment and/or prevention of malaria, which comprises administering to a patient in need of such treatment an effective amount of a compound of formula (I) as defined in claim 1 or an N-oxide thereof, or a pharmaceutically acceptable salt thereof.

16. A compound as claimed in claim 6, wherein Z represents phenyl, naphthyl, furyl, benzofuryl, pyrrolyl, indolyl, pyrazolyl, imidazolyl, imidazo[1,2-a]pyridinyl, imidazo[1,5-a]pyridinyl, imidazo[1,2-a]pyrazinyl, oxadiazolyl, [1,2,4]-triazolo[4,3-a]pyridinyl, tetrazolo[1,5-a]pyridinyl, pyridinyl, quinolinyl, naphthyridinyl, pyridazinyl, cinnolinyl, pyrimidinyl, pyrazinyl or quinoxalinyl, any of which groups may be optionally substituted by one, two or three substituents independently selected from halogen, cyano, C₁₋₆ alkyl, trifluoromethyl, C₂₋₆ alkynyl, cyclopropyl, C₁₋₆ alkoxy, difluoromethoxy, trifluoromethoxy, trifluoroethoxy, methylenedioxy, C₁₋₆ alkylsulfonyl, di(C₁₋₆)alkylamino, morpholinyl, pyrazolyl, imidazolyl and (C₁₋₆)alkylimidazolyl.

17. A compound as claimed in claim 7, wherein Z represents phenyl, naphthyl, furyl, benzofuryl, pyrrolyl, indolyl, pyrazolyl, imidazolyl, imidazo[1,2-a]pyridinyl, imidazo[1,5-a]pyridinyl, imidazo[1,2-a]pyrazinyl, oxadiazolyl, [1,2,4]-triazolo[4,3-a]pyridinyl, tetrazolo[1,5-a]pyridinyl, pyridinyl, quinolinyl, naphthyridinyl, pyridazinyl, cinnolinyl, pyrimidinyl, pyrazinyl or quinoxalinyl, any of which groups may be optionally substituted by one, two or three substituents independently selected from halogen, cyano, C₁₋₆ alkyl, trifluoromethyl, C₂₋₆ alkynyl, cyclopropyl, C₁₋₆ alkoxy, difluoromethoxy, trifluoromethoxy, trifluoroethoxy, methylenedioxy, C₁₋₆ alkylsulfonyl, di(C₁₋₆)alkylamino, morpholinyl, pyrazolyl, imidazolyl and (C₁₋₆)alkylimidazolyl.

18. A compound as claimed in claim 6 wherein R² represents chloro.

19. A compound as claimed in claim 7 wherein R² represents chloro.

20. A compound as claimed in claim 8 wherein R² represents chloro.

21. A compound as claimed in claim 15 wherein R² represents chloro.

22. A compound as claimed in claim 16 wherein R² represents chloro.

23. A compound as claimed in claim 3 wherein R¹⁶ represents hydrogen, halogen, cyano, trifluoromethyl, C₁₋₆ alkoxy, trifluoromethoxy or C₁₋₆ alkylsulfonyl.

24. A compound as claimed in claim 3 wherein R¹ represents C₃₋₇ cycloalkyl or C₃₋₇ heterocycloalkyl, either of which groups may be optionally substituted by one, two or three substituents independently selected from halogen and C₁₋₆ alkyl.
